(19) United States
(12) Patent Application Publication
(10) Pub. No.: US 2024/0026331 A1
(43) Pub. Date: Jan. 25, 2024

(54) RECOMBINANT NEPROSIN POLYPEPTIDES AND METHODS OF EXPRESSION

(71) Applicant: Codexis, Inc., Redwood City, CA (US)

(72) Inventors: Arianna I. Celis Luna, Los Angeles, CA (US); Da Duan, Foster City, CA (US); Paul Campion Jordan, Corte Madera, CA (US); Kathleen Susan Molnar, San Francisco, CA (US); Adam P. Silverman, San Carlos, CA (US); Tricia Windgassen, Redwood City, CA (US)

(21) Appl. No.: 18/315,466

(22) Filed: May 10, 2023

Related U.S. Application Data
(60) Provisional application No. 63/340,411, filed on May 10, 2022.
(60) Provisional application No. 63/356,181, filed on Jun. 28, 2022.

(51) Int. Cl. C12N 9/50; A61P 1/14

(52) U.S. Cl. CPC C12N 9/63; C12Y 304/21026; A61P 1/14; C07K 2319/02; A61K 38/00

(57) ABSTRACT

The present disclosure relates to methods of expressing recombinant neprosin polypeptide, host cells and expression vectors for expressing the recombinant neprosin polypeptide, and methods of using the expressed neprosin polypeptide for therapeutic and other purposes.

### CROSS-REFERENCE TO RELATED APPLICATIONS

This application claims the benefit under 35 U.S.C. § 119(e) of U.S. Provisional Application No. 63/340,411, filed May 10, 2022, and U.S. Provisional Application No. 63/356,181, filed Jun. 28, 2022, the contents of all of which are incorporated by reference herein.

### REFERENCE TO SEQUENCE LISTING, TABLE OR COMPUTER PROGRAM

The Sequence Listing concurrently submitted herewith as file name CX7-229US3_ST26.txt, created on May 10, 2023, with a file size of 187,597 bytes, is part of the specification and is incorporated by reference herein.

### TECHNICAL FIELD

The present disclosure relates to recombinant neprosin polypeptides, polynucleotides encoding the recombinant neprosin polypeptides, and methods of expressing the recombinant polynucleotides. Further provided are use of the recombinant neprosin polypeptides for therapeutic and other purposes.

### BACKGROUND

Celiac disease is a chronic autoimmune disorder typically affecting the small intestine. Symptoms of the disease are caused by a reaction to gluten proteins and other storage proteins in consumed grain products (e.g., serpins, purinins, etc.). Clinically, the disease is detectable in part through the quantitation of antibodies specific for gluten and tissue transglutaminase (tTG). The autoimmune responses result in development of small intestinal mucosal villous atrophy with crypt hyperplasia and mucosal inflammation. Symptoms of celiac disease can vary from individual to individual, and may include one or more of fatigue, chronic diarrhea, constipation, malabsorption of nutrients, weight loss, abdominal distension, anemia, as well as a substantially enhanced risk for the development of osteoporosis and intestinal malignancies (e.g., lymphoma and carcinoma).

Celiac disease is also a risk factor for other diseases. For example, autism is associated with celiac disease, and a gluten-free diet may help alleviate some symptoms of autism. Similarly, it is believed that some people with attention deficit hyperactivity disorder exhibit fewer symptoms when gluten is removed from their diets. Other conditions that may benefit from elimination of dietary gluten include rheumatoid arthritis and fibromyalgia.

Treatment for gluten intolerance, particularly celiac disease, commonly involves a lifelong, strict gluten-free diet. However, gluten-free diet is inconvenient, restrictive, and gluten is difficult to avoid. Proteases that act on gluten have been proposed as a potential therapeutic for reducing the inflammatory effects of gluten and moderating its role in celiac disease. One such protease is the neprosin class of prolyl endopeptidases (PEP) found in the insectivorous tropical pitcher plants of Nepenthes species. Neprosin can hydrolyze proline-rich gliadin, which along with glutenin are the two main proteins of gluten and are associated with triggering celiac disease in susceptible individuals. Small quantities of neprosin are present in the Nepenthes pitcher plants and thus other sources of neprosin are desirable for assessing the protease in therapeutic applications.

### SUMMARY

The present disclosure provides recombinant neprosin polypeptides, methods of expressing the recombinant neprosin polypeptide that result in efficient expression of neprosin, particularly in soluble form, and use of the recombinant neprosin polypeptides in treating diseases or conditions associated with gluten intolerance, such as celiac disease.

In one aspect, the present disclosure provides a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to the pre-pro-polypeptide, pro-polypeptide, mature polypeptide, or polypeptide of SEQ ID NO: 1, wherein the amino acid sequence comprises at least at amino acid position 68, 145, 152, or 253, or combinations thereof, and equivalent positions thereof, a substitution with an amino acid other than asparagine (N), wherein the amino acid positions are relative to the reference sequence corresponding to SEQ ID NO: 1.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 105 to 356 of SEQ ID NO: 8, or to the reference sequence corresponding to SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8.

In some embodiments, the amino acid sequence of the recombinant neprosin polypeptide comprises at least a substitution at amino acid position 44 with an amino acid other than asparagine (N). In some embodiments, the amino acid sequence of the recombinant neprosin polypeptide comprises SEQ ID NO: 10.

In some embodiments, the amino acid sequence of the recombinant neprosin polypeptide comprises at least a substitution at amino acid position 121 with an amino acid other than asparagine (N). In some embodiments, the amino acid sequence of the recombinant neprosin polypeptide comprises residues 105 to 356 of SEQ ID NO: 12, or comprises SEQ ID NO: 12.

In some embodiments, the amino acid sequence of the recombinant neprosin polypeptide comprises at least a substitution at amino acid position 128 with an amino acid other than asparagine (N). In some embodiments, the amino acid sequence of the recombinant neprosin polypeptide comprises residues 105 to 356 of SEQ ID NO: 14, or comprises SEQ ID NO: 14.

In some embodiments, the amino acid sequence of the recombinant neprosin polypeptide comprises at least a substitution at amino acid position 229 with an amino acid other than asparagine (N). In some embodiments, the amino acid sequence of the recombinant neprosin polypeptide comprises residues 105 to 356 of SEQ ID NO: 16, or comprises SEQ ID NO: 16.

In some embodiments, the amino acid sequence of the recombinant neprosin polypeptide comprises at least a substitution at amino acid positions 121 and 229 with an amino acid other than asparagine (N). In some embodiments, the amino acid sequence of the recombinant neprosin polypeptide comprises residues 105 to 356 of SEQ ID NO: 18, or comprises SEQ ID NO: 18.

In some embodiments, the amino acid sequence of the recombinant neprosin polypeptide comprises at least a substitution at amino acid positions 44, 128, and 229 with an amino acid other than asparagine (N). In some embodiments, the amino acid sequence of the recombinant neprosin polypeptide comprises residues 105 to 356 of SEQ ID NO: 20, or comprises SEQ ID NO: 20.

In some embodiments, the amino acid sequence of the recombinant neprosin polypeptide comprises at least a substitution at amino acid positions 44, 121, 128, and 229 with an amino acid other than asparagine (N). In some embodiments, the amino acid sequence of the recombinant neprosin polypeptide comprises residues 105 to 356 of SEQ ID NO: 22, or comprises SEQ ID NO: 22.

In some embodiments, the amino acid sequence of the recombinant neprosin polypeptide comprises at least a substitution at amino acid positions 44 and 229 with an amino acid other than asparagine (N). In some embodiments, the amino acid sequence of the recombinant neprosin polypeptide comprises residues 105 to 356 of SEQ ID NO: 32, or comprises SEQ ID NO: 32.

In some embodiments, the amino acid sequence of the recombinant neprosin polypeptide further comprises a signal sequence. In some embodiments, the signal sequence is a heterologous signal sequence. In some embodiments, the signal sequence is functional in a mammalian cell, insect cell, or fungal cell.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence further comprises a signal sequence functional in a mammalian cell, insect cell, or fungal cell. In some embodiments, the amino acid sequence of the recombinant neprosin polypeptide comprises residues 105 to 356 of SEQ ID NO: 8, or comprises SEQ ID NO: 8.

In some embodiments, the recombinant neprosin polypeptide is glycosylated. In some embodiments, the recombinant neprosin polypeptide is not glycosylated on at least at amino acid position 44, 121, 128, or 229, wherein the amino acid positions are relative to SEQ ID NO: 8. In some embodiments, the recombinant neprosin polypeptide is glycosylated but is not glycosylated on at least at amino acid position 44, 121, 128, or 229, wherein the amino acid positions are relative to SEQ ID NO: 8. In some embodiments, the recombinant neprosin polypeptide is not glycosylated.

In another aspect, the present disclosure provides a recombinant polynucleotide comprising a polynucleotide sequence encoding a recombinant neprosin polypeptide described herein. In some embodiments, the polynucleotide sequence is codon optimized, preferably for expression in a mammalian cell, insect cell, or fungal cell.

In another aspect, the present disclosure provides expression vectors comprising a recombinant polynucleotide encoding a recombinant neprosin polypeptide. In some embodiments, the expression vector comprises a control sequence, such as a promoter preferably for expression of the recombinant neprosin polypeptide in mammalian cells, insect cells, or fungal cells.

In another aspect, further provided are host cells comprising the expression vectors. In some embodiments, the host cell is a eukaryotic cell, preferably a mammalian cell, insect cell, or fungal cell.

In some embodiments, the host cell is a mammalian cell comprising an expression vector comprising a polynucleotide sequence encoding a recombinant neprosin polypeptide. In some embodiments, the mammalian cell is a human cell or rodent cell. In some embodiments, the mammalian cell is Expi293, HeLa, U205, A549, HT1080, CAD, P19, NIH 3T3, L929, Hek 293, 293F, 293E, 293T, COS, Vero, NS0 cell, Sp2/0 cell, DUKX-X11, MCF-7, Y79, SO-Rb50, Hep G2, J558L, or CHO cell. In some embodiments, the polynucleotide sequence encoding the recombinant neprosin polypeptide is codon optimized for expression in a mammalian cell.

In some embodiments, the host cell comprises an insect cell comprising an expression vector comprising a polynucleotide sequence encoding a recombinant neprosin polypeptide. In some embodiments, the insect cell is a lepidopteran or dipteran insect cell. In some embodiments, the insect cell is Sf9 cell, Sf21 cell, Schneider 2 or High Five cell. In some embodiments, the polynucleotide sequence encoding the recombinant neprosin polypeptide is codon optimized for expression in an insect cell.

In some embodiments, the host cell comprises a fungal cell comprising an expression vector comprising a polynucleotide sequence encoding a recombinant neprosin polypeptide. In some embodiments, the fungal cell is a filamentous fungal cell. In some embodiments, the fungal cell is a yeast cell. In some embodiments, the fungal cell is of Pichia, Saccharomyces, Yarrowia, Kluyveromyces, Aspergillus, Trichoderma, Neurospora, Mucor, Penicillium, Trichoderma, or Myceliophthora. In some embodiments, the polynucleotide sequence encoding the recombinant neprosin polypeptide is codon optimized for expression in a fungal cell.

In another aspect, the present disclosure provides a method of expressing a recombinant neprosin polypeptide, the method comprising culturing a host cell comprising an expression vector comprising a polynucleotide sequence encoding a recombinant neprosin polypeptide under suitable conditions such that the recombinant neprosin polypeptide is expressed.

In some embodiments of the method, the expressed recombinant neprosin polypeptide is a mature neprosin polypeptide. In some embodiments, the expressed recombinant neprosin polypeptide is a pro-polypeptide/pro-enzyme form of neprosin polypeptide. In some embodiments, the expressed recombinant neprosin polypeptide is a pre-pro-polypeptide/pre-pro-enzyme form of neprosin polypeptide. In some embodiments, the expressed recombinant neprosin polypeptide is secreted. In some embodiments, the recombinant neprosin polypeptide is expressed in soluble form.

In some embodiments, the expressed recombinant neprosin polypeptide is glycosylated. In some embodiments, the expressed recombinant neprosin polypeptide is not glycosylated on at least at amino acid position 44, 121, 128, or 229, wherein the amino acid positions are relative to SEQ ID NO: 8. In some embodiments, the expressed recombinant neprosin polypeptide is glycosylated but is not glycosylated on at least at amino acid position 44, 121, 128, or 229, wherein the amino acid positions are relative to SEQ ID NO: 8. In some embodiments, the expressed recombinant neprosin polypeptide is non-glycosylated.

In another aspect, the present disclosure provides a method of preparing a mature neprosin polypeptide, particularly under in vitro conditions, the method comprising treating a full-length neprosin polypeptide, or a pre-pro-polypeptide or pro-polypeptide of the neprosin polypeptide under acidic conditions suitable for formation of mature neprosin polypeptide. In some embodiments, the acidic conditions is a pH of about 2 to 6, or a pH of about 2 to about 4.5, preferably a pH of about 2 to about 3. In some embodiments, the mature neprosin polypeptide is prepared by proteolytic cleavage. In some embodiments, the mature neprosin prepared by the method is proteolytically active.

In a further aspect, the present disclosure provides a pharmaceutical composition comprising the recombinant neprosin polypeptide, or pro-polypeptide or mature polypeptide form of the recombinant neprosin polypeptide. In some embodiments, the pharmaceutical composition further comprises a pharmaceutically acceptable excipient and/or carrier.

In another aspect, the present disclosure provides a method of treating or preventing symptoms of a disease or condition associated with gluten intolerance, the method comprising administering to a subject in need thereof an effective amount of a recombinant neprosin polypeptide described herein or a pharmaceutical composition thereof. In some embodiments, the recombinant neprosin polypeptide or pharmaceutical composition thereof is administered prior to, concurrently with, or subsequent to consumption of gluten-containing food. In some embodiments, the disease or condition associated with gluten intolerance in celiac disease.

### BRIEF DESCRIPTION OF THE DRAWINGS

FIG. 1A depicts the amino acid sequence of neprosin polypeptide of SEQ ID NO: 1 and the proposed domains of the signal peptide, activation peptide, mature neprosin, along with the glycosylation sites at amino acid positions 68, 145, 152, and 253 relative to SEQ ID NO: 1. FIG. 1B depicts the amino acid sequence of the neprosin pro-polypeptide of SEQ ID NO: 8 and the pro- and mature neprosin protease regions. The glycosylation sites at amino acid positions 44, 121, 128, and 229 relative to SEQ ID NO: 8, equivalent to amino acid positions 68, 145, 152, and 253 of SEQ ID NO: 1, are also shown.

FIG. 2 shows a photograph of SDS PAGE analysis of formation of activated neprosin protease polypeptide at pH 2.5 for neprosin expressed in CHO cells (Panel A) and neprosin expressed in Expi293 cells. The activated neprosin polypeptide appears as about a 25.5 kDa polypeptide in the SDS PAGE system used.

FIG. 3 shows a photograph of SDS PAGE analysis of formation of proteolytically active mature neprosin polypeptide under conditions of (i) different pHs, (ii) unbuffered Simulated Gastric Fluid (SGF), (pH 7) or (iii) unbuffered SGF+pepsin, as analyzed by SDS PAGE. Panel A is an SDS-PAGE of neprosin protein dialyzed in buffer at different pHs, SGF, or SGF+pepsin. Panel B is an SDS-PAGE of neprosin protein following 5 h dialysis.

FIG. 4 provides a graph comparing proteolytic activity on gliadin substrate of mature, activated neprosin polypeptide compared to neprosin polypeptide prepared from cells expressing mSP-His-Npr1.

FIG. 5 provides a photograph of SDS PAGE analysis of recombinant neprosin pro-polypeptides expressed in Pichia cells for wild-type (WT) construct (SEQ ID NO: 56), N216A mutant construct (SEQ ID NO: 60), and N216A/N324A mutant construct (SEQ ID NO: 66). The WT, N216A, and N216A/N324A neprosin pro-polypeptide forms appear as heterogenous mixtures of glycosylated polypeptides as evidenced by smears around 60 kDa on the SDS-PAGE gel Smaller molecular weight bands around 29 kDa correspond to the activated forms of each of these proteins, which are generated during expression and purification. Amino acid positions 216 and 324 of SEQ ID NO: 60 and SEQ ID NO: 66 are equivalent to amino acid positions 121 and 229, respectively, of SEQ ID NO: 8.

FIG. 6 provides a photograph of SDS-PAGE analysis of the formation of activated neprosin protease polypeptide at pH 2.5 for the WT construct (SEQ ID NO: 56), N216A mutant construct (SEQ ID NO: 60), and N216A/N324A mutant construct (SEQ ID NO: 66) expressed in Pichia cells. The activated WT neprosin appears as two glycosylated polypeptides around 29 and 35 kDa. Activated N216A neprosin and N216A/N324A neprosin appear as single glycosylated species around 29 kDa.

FIG. 7 provides a graph of proteolytic activity on gliadin substrate in buffered SGF conditions (pH 3, 0.8 mg/mL pepsin) of activated neprosin Npr1 prepared from Npr1 expressed in Pichia cells compared to activated Npr1 prepared from Npr1 expressed in Chinese Hamster Ovary (CHO) cells or no added neprosin enzyme. Activity by the pepsin included in SGF on the gliadin substrate is responsible for the decrease in gliadin levels observed in the “no enzyme” sample.

FIG. 8 provides a graph of proteolytic activity on gliadin substrate in buffered SGF conditions (pH 3, 0.8 mg/mL pepsin) of activated N216A Npr1 (SEQ ID NO: 60) and N216A/N324A Npr1 (SEQ ID NO: 66) prepared from the recombinant neprosin polypeptides expressed in Pichia cells compared to activated WT Npr1 prepared from Npr1 expressed in Pichia cells, or no added neprosin enzyme. Activity by the pepsin included in SGF on the gliadin substrate is responsible for the decrease in gliadin levels observed in the “no enzyme” sample.

FIG. 9 provides a graph of dose-dependent proteolytic activity of activated WT Npr1 prepared from Npr1 expressed in CHO cells on gliadin substrate inside a mouse stomach. Data is shown as mean±standard deviation. Statistical test applied is Dunnett's T3 multiple comparisons test where: *p≤0.05, **p≤0.01, ***p≤0.001, and ****p≤0.0001.

FIG. 10 provides a graph showing levels of T cell activation in peripheral blood mononuclear cells (PBMC) collected from five human patients with celiac disease and challenged with gluten treated or not treated with activated WT Npr1 prepared from Npr1 expressed in CHO cells. Activated WT Npr1 activity in a gastric digest was tested at 15- and 60-min. Positive control data is digested and deamidated gliadin (D-Gliadin) with buffer as the vehicle control (60-min gastric digest). Data shown as mean±standard deviation. Statistical test applied is a Friedman's test where: *p≤0.05, **p≤0.01, and ***p≤0.001.

### DETAILED DESCRIPTION

The present disclosure provides recombinant neprosin polypeptides, recombinant polynucleotides encoding the recombinant neprosin polypeptides, host cells comprising the recombinant polynucleotides, and methods of expressing the recombinant neprosin polypeptides using the host cells. The present disclosure further provides recombinant neprosin polypeptides prepared from the host cells, particularly in soluble form, and methods of preparing proteolytically active forms of the recombinant neprosin for accessing therapeutic efficacy and use in the treatment of gluten intolerance.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole. All patents, patent applications, articles and publications mentioned herein, both supra and infra, are hereby expressly incorporated herein by reference.

As used herein, the singular “a”, “an,” and “the” include the plural references, unless the context clearly indicates otherwise.

As used herein, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Polynucleotide” is used herein to denote a polymer comprising at least two nucleotides where the nucleotides are either deoxyribonucleotides or ribonucleotides. The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Duplex” and “ds” refer to a double-stranded nucleic acid (e.g., DNA) molecule comprised of two single-stranded polynucleotides that are complementary in their sequence (A pairs to T, C pairs to G), arranged in an antiparallel 5′ to 3′ orientation, and held together by hydrogen bonds between the nucleobases (i.e., adenine [A], guanine [G], cytosine [C], and thymine [T]).

“Protein,” “polypeptide,” and “peptide” are used interchangeably to denote a polymer of at least two amino acids covalently linked by an amide bond, regardless of length or post-translational modification (e.g., glycosylation or phosphorylation). Unless indicated otherwise, amino acid sequences are written left to right in amino to carboxy orientation.

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Ca). For example, whereas “Ala” designates alanine without specifying the configuration about the α carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more genes that originally encoded separate proteins. In some embodiments, fusion proteins are created by recombinant technology (e.g., molecular biology techniques known in the art).

“Neprosin” refers to prolyl endoproteases initially isolated from the pitcher secretions of Nepenthes species. Neprosin is known to cleave proteins carboxy-terminal to proline, with high specificity. The enzyme is active at about pH 2 to about pH 5. Neprosin, as used herein, includes isoforms, isotypes, homologs, orthologs, variants, and recombinants neprosin, and salts thereof. Exemplary neprosin polypeptides are disclosed in WO2015192211 and Ting et al., Plant Physiol Biochem., 2022, 183:23-35, incorporated herein by reference.

“Nepenthesin” refers to an aspartic protease having the Enzyme Commission number EC 3.4.23.12, and includes all isoforms, isotypes, and variants of nepenthesin such as nepenthesin I and nepenthesin II, nepenthesin isoforms, and recombinant nepenthesin, and salts thereof. Nepenthesin is an aspartic protease of plant origin that can be isolated or concentrated from a variety of plant sources, such as the pitcher secretions of Nepenthes, commonly known as monkey cups in tropical regions. Nepenthesin is described in International patent publications WO2014078935 and WO2014138927, which are incorporated herein by reference in its entirety.

“Mature protein” or “mature polypeptide” refers to the final processed biological protein or polypeptide or product.

“Pro-protein,” “pro-polypeptide,” or “pro-peptide” refers to a precursor protein, polypeptide, or peptide that is processed by post-translational modification, to form a biologically active protein, polypeptide, or peptide. In some embodiments, the post translational modification is a cleavage reaction to form the protein, polypeptide, or peptide. “Pro-enzyme” refers to a precursor polypeptide that is processed by post-translational modification, in particular a cleavage reaction, to form an active enzyme.

“Pre-pro-protein,” “pre-pro-polypeptide,” or “pre-pro-peptide” refers to a precursor protein, polypeptide, or peptide that includes a signal sequence and which can be processed by posttranslational modification, in particular a cleavage reaction, to generate a pro-protein, pro-polypeptide, or pro-peptide. Generally, a cleavage reaction removes a signal sequence to generate a pro-protein, pro-polypeptide, or pro-peptide. “Pre-pro-enzyme” refers to a precursor protein, polypeptide, or peptide that is processed by post-translational modification, in particular a cleavage reaction that removes a signal sequence, to form a pro-enzyme.

“Full-length” in context of a protein or polypeptide refers to the protein or polypeptide which is not processed to alter the amino acid sequence of the entire protein or polypeptide. For example, a full-length protein is the entire protein encoded in the corresponding mRNA.

“Engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted (e.g., by the local homology algorithm of Smith and Waterman; Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA., 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et at, J. Mol. Biol., 1990, 215:403-410; and Altschul et al., Nucleic Acids Res., 1977, 25:3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full-length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a recombinant neprosin polypeptide, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X121 as compared to SEQ ID NO: 8” (or a “residue difference at position 121 as compared to SEQ ID NO: 8”) refers to a difference of the amino acid residue at the polypeptide position corresponding to position 121 of SEQ ID NO: 8. Thus, if the reference polypeptide of SEQ ID NO: 8 has an asparagine at position 121, then a “residue difference at position X121 as compared to SEQ ID NO: 8” refers to an amino acid substitution with any residue other than asparagine at the position of the polypeptide corresponding to position 121 of SEQ ID NO: 8. In some instances herein, the specific amino acid residue difference at a position is indicated as “XnY” where “Xn” specifies the corresponding residue and position of the reference polypeptide (as described above), and “Y” is the single letter identifier of the amino acid found in the engineered or recombinant polypeptide (i.e., the different residue than in the reference polypeptide). In some embodiments, the amino acid difference, e.g., a substitution, is denoted by the abbreviation “nY,” without the identifier for the residue in the reference sequence.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the properties of a neprosin polypeptide. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full-length recombinant neprosin polypeptide) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant neprosin polypeptides may be present within a cell, present in the cellular medium, or prepared in various forms, such as lysates or isolated preparations. As such, in some embodiments, the recombinant neprosin polypeptides provided herein are isolated polypeptides.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure neprosin polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant neprosin polypeptides are substantially pure polypeptide compositions.

“Proteolytic activity” and “proteolysis” are used interchangeably herein refer to the breakdown of proteins into smaller polypeptides or amino acids. The breakdown of proteins is generally the result of hydrolysis of the peptide bond by a protease (proteinase) enzyme. Exemplary protease enzymes include but are not limited to neprosin, nepenthesin, pepsin, trypsin, chymotrypsin, elastase; carboxypeptidase A and B, and peptidases (e.g., amino peptidase, dipeptidase and enteropeptidase).

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the neprosin polypeptide are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, pro-peptide sequences, promoter sequences, signal peptide sequences, initiation sequences, and transcription terminators. At a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Vector” refers to a polynucleotide construct for introducing a polynucleotide sequence into a cell. In some embodiments, the vector is an expression vector that is operably linked to a suitable control sequence capable of effecting the expression in a suitable host of the polypeptide encoded in the polynucleotide sequence. In some embodiments, an “expression vector” has a promoter sequence operably linked to the polynucleotide sequence (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Culturing” refers to the growing of a population of cells, such as host cells, under suitable conditions using any suitable medium (e.g., liquid, gel, or solid).

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Host cell” and “host strain” refer to suitable hosts for expression vectors comprising DNA provided herein (e.g., a polynucleotide sequences encoding at least one neprosin polypeptide). In some embodiments, the host cells are prokaryotic or eukaryotic cells that have been transformed or transfected with vectors constructed using recombinant DNA techniques as known in the art.

“Effective amount” means an amount sufficient to produce the desired result. One of general skill in the art may determine what the effective amount by using routine experimentation.

“Isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

“Subject” encompasses mammals such as humans, non-human primates, livestock, companion animals, and laboratory animals (e.g., rodents and lagamorphs). It is intended that the term encompass females as well as males. In some embodiments, a “patient” means any subject that is being assessed for, treated for, or is experiencing disease.

“Administration” and “administering” a composition mean providing a composition of the present invention to a subject (e.g., to a person suffering from the effects of gluten sensitivity, such as celiac disease).

“Pharmaceutically acceptable” means a material that can be administered to a subject without causing any undesirable biological effects or interacting in a deleterious manner with any of the components in which it is contained and that possesses the desired biological activity.

“Excipient” refers to any pharmaceutically acceptable additive, carrier, diluent, adjuvant, or other ingredient, other than the active pharmaceutical ingredient. Excipients are typically included for formulation and/or administration purposes.

“Carrier” when used in reference to a pharmaceutical composition means any of the standard pharmaceutical carrier, buffers, and excipients, such as stabilizers, preservatives, and adjuvants.

“Therapeutically effective amount” when used in reference to symptoms of disease/condition refers to the amount and/or concentration of a compound (e.g., recombinant neprosin polypeptides) that ameliorates, attenuates, or eliminates one or more symptom of a disease/condition or prevents or delays the onset of symptom(s). A “therapeutically effective amount” when used in reference to a disease/condition refers to the amount and/or concentration of a composition (e.g., recombinant neprosin polypeptides) that ameliorates, attenuates, or eliminates the disease/condition. In some embodiments, the term is use in reference to the amount of a composition that elicits the biological (e.g., medical) response by a tissue, system, or animal subject that is sought by the researcher, physician, veterinarian, or other clinician.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable by one of ordinary skill in the art.

“Concurrent administration,” or “co-treatment,” as used herein includes administration of the agents together, or before or after each other.

“Modulate,” “attenuate” or “ameliorate” means any treatment of a disease or disorder in a subject, such as a mammal, including: preventing or protecting against the disease or disorder, e.g., causing the abnormal biological reaction or symptoms not to develop; inhibiting the disease or disorder, arresting or suppressing the development of abnormal biological reactions and/or clinical symptoms; and/or relieving the disease or disorder, e.g., causing the regression of abnormal biological reactions and/or symptoms.

“Preventing” or “inhibiting” refers to the prophylactic treatment of a subject in need thereof. The prophylactic treatment can be accomplished by providing an appropriate dose of a therapeutic agent to a subject at risk of suffering from an ailment, thereby substantially averting onset of the ailment.

“Antigenic food or protein” as used herein refers to a food containing protein that can cause an immune and/or inflammatory response in the intestine of a sensitive individual. In a preferred embodiment, the individual is a human and the food is a food intended for human consumption. Antigenic foods include, by way of example and not limitation, wheat, rye, barley, peanuts, nuts and seeds. In some embodiments, antigenic proteins from these foods include prolamin proteins, 2S albumins, non-specific lipid transfer proteins, bifunctional α-amylase/protease inhibitors, soybean hydrophobic protein, indolines, gluten, serpins, purinins, alpha-amylase/protease inhibitors, globulins, and farinins. In some embodiments, the antigenic protein (or peptide) is rich in proline and/or glutamine residues. In a preferred embodiment, the antigenic protein is gluten. In some embodiments, the potentially antigenic protein is a wheat protein.

“Gluten” generally refers to the proteins present in wheat or related grain species, including barley and rye, which have potential harmful effect to certain individuals. Gluten proteins include gliadins such as a-gliadins, β-gliadins, γ-gliadins and w-gliadins, which are monomelic proteins, and glutenins, which are highly heterogeneous mixtures of aggregates of high-molecular-weight and low-molecular-weight subunits held together by disulfide bonds. Many wheat gluten proteins have been characterized (see, e.g., Woychik et al., Amino Acid Composition of Proteins in Wheat Gluten, 1 Agric. Food Chem., 1961, 9(4):307-310). The term gluten as used herein also includes oligopeptides that can be derived from normal human digestion of gluten proteins from gluten containing foods and cause the abnormal immune response. Some of these oligopeptides are resistant to normal digestive enzymes. Gluten, including the above-mentioned proteins and oligopeptides, is believed to act as an antigen for T cells (e.g., IELs) in patients with gluten intolerance (e.g., celiac sprue). The term gluten also refers to denatured gluten, such as would be found in baked products.

“Gluten sensitivity and related conditions” refers to any condition stemming from intolerance or sensitivity to gluten proteins or peptides. These include, without limitation, celiac sprue (celiac disease), wheat allergy, gluten sensitivity, gluten-sensitive enteropathy, idiopathic gluten sensitivity, and dermatitis herpetiformis. Related conditions also include, without limitation, autism, attention deficit hyperactivity disorder (ADHD), rheumatoid arthritis, fibromyalgia, Crohn's disease, nutrient malabsorption, and irritable bowel syndrome (IBS).

“Manifestations of celiac disease” refers to any of the symptoms or clinical presentations of celiac disease. Such manifestations include, without limitation, intestinal inflammation, “foggy mind”, depression, anxiety, ADHD-like behavior, abdominal pain, bloating, diarrhea, constipation, headaches, migraines, bone or joint pain, chronic fatigue, small intestine damage, development of tissue transglutaminase (tTG) antibodies, severe acne, vomiting, weight loss, irritability, iron-deficiency anemia, arthritis, tingling numbness in the extremities, infertility, and canker sores of the mouth. Manifestations further include small intestinal mucosal villous atrophy with crypt hyperplasia, mucosal inflammation of the intestine, malabsorption of nutrients, abdominal distension, as well as a substantially enhanced risk for the development of osteoporosis and intestinal malignancies (lymphoma and carcinoma).

Recombinant Neprosin Polypeptide and Expressed Neprosin Polypeptides

The pitcher secretions of Nepenthes, a carnivorous pitcher plant commonly known as monkey cups that grow in tropical regions, contain a number of different proteases. Concentrated Nepenthes pitcher fluid has high specificity for proline- and glutamine-rich gluten peptides. U.S. patent publication Nos. 2014/0186330 and 2014/0140980, incorporated herein by reference in their entireties, describe the activity and specificity of concentrated Nepenthes pitcher fluid and recombinant Nepenthes enzymes. The pitcher fluid is acidic, and the enzymes therein are generally most active at acidic pH.

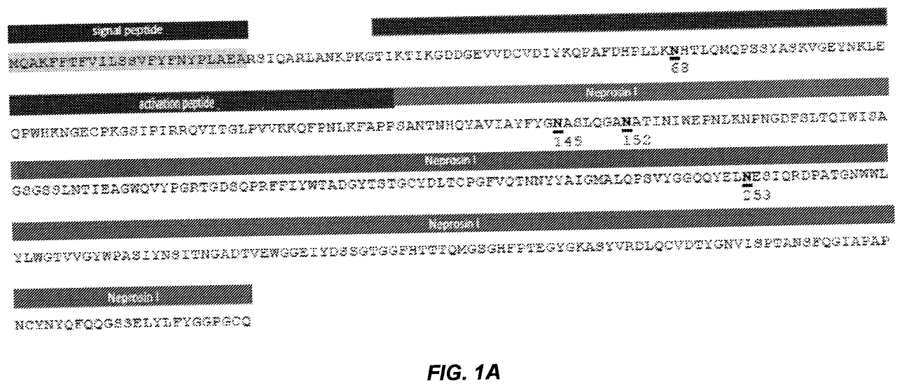

The prolyl-endoprotease neprosin. one of the proteases present in the pitcher secretions, possesses high proteolytic activity for cleaving proline-rich proteins and oligopeptides, such as gluten proteins. The amino acid sequence of the neprosin polypeptide (SEQ ID NO: 1) present in N. x ventrata is shown in FIG. 1A. The neprosin polypeptide includes a signal sequence, a pro-domain region, and a neprosin protease region. Analysis of the pH activated mature, proteolytically active neprosin polypeptide by N-terminal sequencing, as disclosed herein, indicates that the pro-polypeptide is cleaved at acidic pH between proline and serine residues at amino acid positions 128 and 129 of SEQ ID NO: 1 such that the primary form of the mature, proteolytically active neprosin polypeptide generated at acidic pH begins at the serine residue at amino acid position 129. Without being bound by any theory of operation, N-glycosylation sites are shown in FIG. 1A at amino acid positions 68, 145, 152, and 253 in reference to SEQ ID NO: 1. A pro-polypeptide form of the neprosin polypeptide (SEQ ID NO: 8) is provided in FIG. 1B, with the pro-domain (pro-peptide) and protease region (Neprosin 1) shown by the block arrows. The N-glycosylation sites. i.e., asparagine residue (N), are present at amino acid positions 44, 121, 128, and 229 in reference to SEQ ID NO: 8, which is equivalent to amino acid positions 68, 145, 152, and 253 in reference to SEQ ID NO: 1.

Neprosin is active at a broad pH range, and is especially active at low pH (e.g., about 3 to 5) and cleaves peptides on the carboxy (C)-terminal side of proline. This cleavage appears to be highly specific. The activity and specificity of neprosin in cleaving gluten proteins into peptides that have reduced immunogenicity provides a basis for treating celiac disease and other inflammatory disorders associated with immunogenicity of gluten proteins. Accordingly, the present disclosure provides recombinant neprosin polypeptides and methods of expressing the recombinant neprosin polypeptides, particularly in soluble form. In some embodiments, the recombinant neprosin polypeptide is a pro-polypeptide form. In some embodiments, the pro-polypeptide form can be cleaved, such as under acid pH or by a protease, to form a mature polypeptide.

In some embodiments, the recombinant neprosin in glycosylated. In some embodiments, the amino acid sequence of the recombinant neprosin polypeptide is manipulated to control glycosylation of the neprosin polypeptide. In some embodiments, the pro-polypeptide of the recombinant neprosin polypeptide has altered glycosylation. In some embodiments, the mature polypeptide of the recombinant neprosin polypeptide has an altered glycosylation pattern compared to the naturally occurring polypeptide. In some embodiments, the glycosylation pattern is manipulated by selection of a host cell, e.g., mammalian cell, insect cell, or fungal cell, for expression of the recombinant neprosin polypeptide.

In some embodiments, the recombinant neprosin polypeptide lacks glycosylation at amino acid position 68, 145, 152, or 253, or equivalent positions thereof, and combinations thereof, wherein the amino acid positions are relative to SEQ ID NO: 1. In some embodiments, the recombinant neprosin polypeptide lacks glycosylation at least at amino acid position 68 or equivalent position thereof. In some embodiments, the recombinant neprosin polypeptide lacks glycosylation at least at amino acid position 145 or equivalent position thereof. In some embodiments, the recombinant neprosin polypeptide lacks glycosylation at least at amino acid position 152 or equivalent position thereof. In some embodiments, the recombinant neprosin polypeptide lacks glycosylation at least at amino acid position 253 or equivalent position thereof. In some embodiments, the recombinant neprosin polypeptide is glycosylated but lacks glycosylation on at least at amino acid position 68, 145, 152, or 253, or equivalent position thereof, wherein the amino acid positions are relative to SEQ ID NO: 1.

In some embodiments, the recombinant neprosin polypeptide is non-glycosylated. In some embodiments, the pre-pro-polypeptide of the recombinant neprosin is non-glycosylated. In some embodiments, the pro-polypeptide form of the recombinant neprosin is non-glycosylated. In some embodiments, the mature polypeptide of the recombinant neprosin is non-glycosylated.

In one aspect, the present disclosure provides a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to the pre-pro-polypeptide, pro-polypeptide, mature polypeptide, or polypeptide of SEQ ID NO: 1, wherein the amino acid sequence comprises at least at amino acid position 68, 145, 152, or 253, or combinations thereof, and equivalent positions thereof, a substitution with an amino acid other than asparagine (N), wherein the amino acid positions are relative to the reference sequence corresponding to SEQ ID NO: 1. In some embodiments, the amino acid substitution or amino acid residue at position 68, 145, 152, or 253 is independently selected from A, C, D, E, F, G, H, I, K, L, M, P, Q, R, S, T, V, W, and Y. In some embodiments, the amino acid substitution or amino acid residue at positions 68, 145, 152, and 253 is each A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 68, or equivalent position thereof, a substitution with an amino acid other than asparagine (N), wherein the amino acid position is relative to the reference sequence corresponding to SEQ ID NO: 1. In some embodiments, the amino acid substitution or amino acid residue at position 68 is selected from A, C, D, E, F, G, H, I, K, L, M, P, Q, R, S, T, V, W, and Y. In some embodiments, the amino acid substitution or amino acid residue at position 68 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 145, or equivalent position thereof, a substitution with an amino acid other than asparagine (N), wherein the amino acid position is relative to SEQ ID NO: 1. In some embodiments, the amino acid substitution or amino acid residue at position 145 is selected from A, C, D, E, F, G, H, I, K, L, M, P, Q, R, S, T, V, W, and Y. In some embodiments, the amino acid substitution or amino acid residue at position 145 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 152, or equivalent position thereof, a substitution with an amino acid other than asparagine (N), wherein the amino acid position is relative to SEQ ID NO: 1. In some embodiments, the amino acid substitution or amino acid residue at position 152 is selected from A, C, D, E, F, G, H, I, K, L, M, P, Q, R, S, T, V, W, and Y. In some embodiments, the amino acid substitution or amino acid residue at position 152 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 253, or equivalent position thereof, a substitution with an amino acid other than asparagine (N), wherein the amino acid position is relative to SEQ ID NO: 1. In some embodiments, the amino acid substitution or amino acid residue at position 253 is selected from A, C, D, E, F, G, H, I, K, L, M, P, Q, R, S, T, V, W, and Y. In some embodiments, the amino acid substitution or amino acid residue at position 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 145, or equivalent positions thereof, a substitution with an amino acid other than asparagine (N), wherein the amino acid positions are relative to SEQ ID NO: 1. In some embodiments, the amino acid substitution or amino acid residue at each of positions 68 and 145 is independently selected from A, C, D, E, F, G, H, I, K, L, M, P, Q, R, S, T, V, W, and Y. In some embodiments, the amino acid substitution or amino acid residue at positions 68 and 145 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 152, or equivalent positions thereof, a substitution with an amino acid other than asparagine (N), wherein the amino acid positions are relative to SEQ ID NO: 1. In some embodiments, the amino acid substitution or amino acid residue at each of positions 68 and 152 is independently selected from A, C, D, E, F, G, H, I, K, L, M, P, Q, R, S, T, V, W, and Y. In some embodiments, the amino acid substitution or amino acid residue at positions 68 and 152 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68 and 253 is independently selected from A, C, D, E, F, G, H, I, K, L, M, P, Q, R, S, T, V, W, and Y. In some embodiments, the amino acid substitution or amino acid residue at positions 68 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 145 and 152, or equivalent positions thereof, a substitution with an amino acid other than asparagine (N), wherein the amino acid positions are relative to SEQ ID NO: 1. In some embodiments, the amino acid substitution or amino acid residue at each of positions 145 and 152 is independently selected from A, C, D, E, F, G, H, I, K, L, M, P, Q, R, S, T, V, W, and Y. In some embodiments, the amino acid substitution or amino acid residue at positions 145 and 152 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 145 and 253, or equivalent positions thereof, a substitution with an amino acid other than asparagine (N), wherein the amino acid positions are relative to SEQ ID NO: 1. In some embodiments, the amino acid substitution or amino acid residue at each of positions 145 and 253 is independently selected from A, C, D, E, F, G, H, I, K, L, M, P, Q, R, S, T, V, W, and Y. In some embodiments, the amino acid substitution or amino acid residue at positions 145 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 152 and 253 is independently selected from A, C, D, E, F, G, H, I, K, L, M, P, Q, R, S, T, V, W, and Y. In some embodiments, the amino acid substitution or amino acid residue at positions 152 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145 and 152, or equivalent positions thereof, a substitution with an amino acid other than asparagine (N), wherein the amino acid positions are relative to SEQ ID NO: 1. In some embodiments, the amino acid substitution or amino acid residue at each of positions 68, 145 and 152 is independently selected from A, C, D, E, F, G, H, I, K, L, M, P, Q, R, S, T, V, W, and Y. In some embodiments, the amino acid substitution or amino acid residue at positions 68, 145 and 152 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 145 and 253 is independently selected from A, C, D, E, F, G, H, I, K, L, M, P, Q, R, S, T, V, W, and Y. In some embodiments, the amino acid substitution or amino acid residue at positions 68, 145 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 152 and 253 is independently selected from A, C, D, E, F, G, H, I, K, L, M, P, Q, R, S, T, V, W, and Y. In some embodiments, the amino acid substitution or amino acid residue at positions 68, 152 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 145, 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 145, 152 and 253 is independently selected from A, C, D, E, F, G, H, I, K, L, M, P, Q, R, S, T, V, W, and Y. In some embodiments, the amino acid substitution or amino acid residue at positions 145, 152 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145, 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 145, 152 and 253 is independently selected from A, C, D, E, F, G, H, I, K, L, M, P, Q, R, S, T, V, W, and Y. In some embodiments, the amino acid substitution or amino acid residue at positions 68, 145, 152 and 253 is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N68A, N145A, N152A, or N253A, or equivalent positions thereof, and combinations thereof, wherein the amino acid positions are relative to SEQ ID NO: 1.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N68A, or equivalent position thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N145A, or equivalent position thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N152A, or equivalent position thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A and N152A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to the pre-pro-polypeptide, pro-polypeptide, mature polypeptide, or polypeptide of SEQ ID NO: 1, wherein the amino acid sequence comprises at least substitutions N145A and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N152A and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A, N152A, and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N145A, N152A, and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A, N145A, N152A, and N253A, or equivalent positions thereof.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the polypeptide sequence comprising residues 26 to 381 of SEQ ID NO: 44, or a pro-polypeptide or mature polypeptide thereof, wherein the amino acid at position 68, 152, and/or 253 is other than an asparagine (N).

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the polypeptide sequence comprising residues 26 to 381 of SEQ ID NO: 46, or a pro-polypeptide or mature polypeptide thereof, wherein the amino acid at position 68 is other than an asparagine (N).

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to the polypeptide sequence comprising residues 26 to 381 of SEQ ID NO: 48, or a pro-polypeptide or mature polypeptide thereof, wherein the amino acid at position 152 is other than an asparagine (N).

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the polypeptide sequence comprising residues 26 to 381 of SEQ ID NO: 50, or a pro-polypeptide or mature polypeptide thereof, wherein the amino acid at position 253 is other than an asparagine (N).

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the polypeptide sequence comprising residues 26 to 381 of SEQ ID NO: 52, or a pro-polypeptide or mature polypeptide thereof, wherein the amino acid at position 68 and 253 is other than an asparagine (N). In some embodiments, the amino acid at position 68 and 253 is alanine (A).

In some embodiments, the recombinant neprosin polypeptide comprises an amino acid sequence comprising residues 26 to 381 of SEQ ID NO: 44, 46, 48, 50, or 52. In some embodiments, the recombinant neprosin polypeptide comprises a pro-polypeptide of the amino acid sequence comprising amino acid residues 26 to 381 of SEQ ID NO: 44, 46, 48, 50, or 52. In some embodiments, the recombinant neprosin polypeptide comprises a mature polypeptide of the amino acid sequence comprising amino acid residues 26 to 381 of SEQ ID NO: 44, 46, 48, 50, or 52.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 105 to 356 of SEQ ID NO: 8, or to a reference sequence corresponding to SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the recombinant neprosin polypeptide comprises an amino acid sequence comprising residues 105 to 356 of SEQ ID NO: 8, wherein the amino acid sequence comprises at least at amino acid position 121, 128, or 229, or combinations thereof, a substitution with an amino acid other than asparagine (N), wherein the amino acid positions are relative to SEQ ID NO: 8. In some embodiments, the recombinant neprosin polypeptide comprises an amino acid sequence comprising SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 8, or to the reference sequence corresponding to SEQ ID NO: 8, wherein the amino acid sequence comprises at least at amino acid position 44 a substitution with an amino acid other than asparagine (N), wherein the amino acid position is relative to SEQ ID NO: 8. In some embodiments, the amino acid substitution or amino acid residue at amino acid position 44 is selected from A, C, D, E, F, G, H, I, K, L, M, P, Q, R, S, T, V, W, and Y. In some embodiments, the amino acid substitution or amino acid residue at position 44 is A.

In some embodiments, the recombinant neprosin polypeptide comprises an acid activated proteolytically active polypeptide derived from or comprising a subsequence of SEQ ID NO: 10. In some embodiments, the recombinant neprosin polypeptide comprises an amino acid sequence comprising residues 105 to 356 of SEQ ID NO: 10. In some embodiments, the recombinant neprosin polypeptide comprises an amino acid sequence comprising SEQ ID NO: 10. In some embodiments, the recombinant neprosin polypeptide comprising the amino acid sequence comprising residues 105 to 356 of SEQ ID NO: 10 or comprising SEQ ID NO: 10 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 121 a substitution with an amino acid other than asparagine (N), wherein the amino acid position is relative to SEQ ID NO: 8. In some embodiments, the amino acid substitution or amino acid residue at position 121 is selected from A, C, D, E, F, G, H, I, K, L, M, P, Q, R, S, T, V, W, and Y. In some embodiments, the amino acid substitution or amino acid residue at position 121 is A.

In some embodiments, the recombinant neprosin polypeptide comprises an acid activated proteolytically active polypeptide derived from or comprising a subsequence of SEQ ID NO: 12. In some embodiments, the recombinant neprosin polypeptide comprises an amino acid sequence comprising residues 105 to 356 of SEQ ID NO: 12. In some embodiments, the recombinant neprosin polypeptide comprises an amino acid sequence comprising SEQ ID NO: 12. In some embodiments, the recombinant neprosin polypeptide comprising the amino acid sequence comprising residues 105 to 356 of SEQ ID NO: 12 or comprising SEQ ID NO: 12 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 128 a substitution with an amino acid other than asparagine (N), wherein the amino acid position is relative to SEQ ID NO: 8. In some embodiments, the amino acid substitution or amino acid residue at position 128 is selected from A, C, D, E, F, G, H, I, K, L, M, P, Q, R, S, T, V, W, and Y. In some embodiments, the amino acid substitution or amino acid residue at position 128 is A.

In some embodiments, the recombinant neprosin polypeptide comprises an acid activated proteolytically active polypeptide derived from or comprising a subsequence of SEQ ID NO: 14. In some embodiments, the recombinant neprosin polypeptide comprises an amino acid sequence comprising residues 105 to 356 of SEQ ID NO: 14. In some embodiments, the recombinant neprosin polypeptide comprises an amino acid sequence comprising SEQ ID NO: 14. In some embodiments, the recombinant neprosin polypeptide comprising the amino acid sequence comprising residues 105 to 356 of SEQ ID NO: 14 or comprising SEQ ID NO: 14 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 229 a substitution with an amino acid other than asparagine (N), wherein the amino acid position is relative to SEQ ID NO: 8. In some embodiments, the amino acid substitution or amino acid residue at position 229 is selected from A, C, D, E, F, G, H, I, K, L, M, P, Q, R, S, T, V, W, and Y. In some embodiments, the amino acid substitution or amino acid residue at position 229 is A.

In some embodiments, the recombinant neprosin polypeptide comprises an acid activated proteolytically active polypeptide derived from or comprising a subsequence of SEQ ID NO: 16. In some embodiments, the recombinant neprosin polypeptide comprises an amino acid sequence comprising residues 105 to 356 of SEQ ID NO: 16. In some embodiments, the recombinant neprosin polypeptide comprises an amino acid sequence comprising SEQ ID NO: 16. In some embodiments, the recombinant neprosin polypeptide comprising the amino acid sequence comprising residues 105 to 356 of SEQ ID NO: 12 or comprising SEQ ID NO: 16 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 121 and 229 substitutions with an amino acid other than asparagine (N), wherein the amino acid positions are relative to SEQ ID NO: 8. In some embodiments, the amino acid substitution or amino acid residue at each of positions 121 and 229 is independently selected from A, C, D, E, F, G, H, I, K, L, M, P, Q, R, S, T, V, W, and Y. In some embodiments, the amino acid substitution or amino acid residue at position 121 and 229 is A.

In some embodiments, the recombinant neprosin polypeptide comprises an acid activated, proteolytically active polypeptide derived from or comprising a subsequence of SEQ ID NO: 18. In some embodiments, the recombinant neprosin polypeptide comprises an amino acid sequence comprising residues 105 to 356 of SEQ ID NO: 18. In some embodiments, the recombinant neprosin polypeptide comprises an amino acid sequence comprising SEQ ID NO: 18. In some embodiments, the recombinant neprosin polypeptide comprising the amino acid sequence comprising residues 105 to 356 of SEQ ID NO: 18 or comprising SEQ ID NO: 18 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 128, and 229, or equivalent positions thereof, substitutions with an amino acid other than asparagine (N), wherein the amino acid positions are relative to SEQ ID NO: 8. In some embodiments, the amino acid substitutions or amino acid residue at each of positions 44, 128, and 229 is independently selected from A, C, D, E, F, G, H, I, K, L, M, P, Q, R, S, T, V, W, and Y. In some embodiments, the amino acid substitution or amino acid residue at positions 44, 128, and 229 is A.

In some embodiments, the recombinant neprosin polypeptide comprises an acid activated proteolytically active polypeptide derived from or comprising a subsequence of SEQ ID NO: 20. In some embodiments, the recombinant neprosin polypeptide comprises an amino acid sequence comprising residues 105 to 356 of SEQ ID NO: 20. In some embodiments, the recombinant neprosin polypeptide comprises an amino acid sequence comprising SEQ ID NO: 20. In some embodiments, the recombinant neprosin polypeptide comprising the amino acid sequence comprising residues 105 to 356 of SEQ ID NO: 20 or comprising SEQ ID NO: 20 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 121, 128, and 229 substitutions with an amino acid other than asparagine (N), wherein the amino acid positions are relative to SEQ ID NO: 8. In some embodiments, the amino acid substitution or amino acid residue at each of positions 44, 121, 128, and 229 is independently selected from A, C, D, E, F, G, H, I, K, L, M, P, Q, R, S, T, V, W, and Y. In some embodiments, the amino acid substitution or amino acid residue at positions 44, 121, 128, and 229 is A.

In some embodiments, the recombinant neprosin polypeptide comprises an acid activated proteolytically active polypeptide derived from or comprising a subsequence of SEQ ID NO: 22. In some embodiments, the recombinant neprosin polypeptide comprises an amino acid sequence comprising residues 105 to 356 of SEQ ID NO: 22. In some embodiments, the recombinant neprosin polypeptide comprises an amino acid sequence comprising SEQ ID NO: 22. In some embodiments, the recombinant neprosin polypeptide comprising the amino acid sequence comprising residues 105 to 356 of SEQ ID NO: 22 or comprising SEQ ID NO: 22 is not glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44 and 229 a substitution with an amino acid other than asparagine (N), wherein the amino acid positions are relative to SEQ ID NO: 8. In some embodiments, the amino acid substitution or amino acid residue at each of positions 44 and 229 is independently selected from A, C, D, E, F, G, H, I, K, L, M, P, Q, R, S, T, V, W, and Y. In some embodiments, the amino acid substitution or amino acid residue at positions 44 and 229 is A.

In some embodiments, the recombinant neprosin polypeptide comprises an acid activated proteolytically active polypeptide derived from or comprising a subsequence of SEQ ID NO: 32. In some embodiments, the recombinant neprosin polypeptide comprises an amino acid sequence comprising residues 105 to 356 of SEQ ID NO: 32. In some embodiments, the recombinant neprosin polypeptide comprises an amino acid sequence comprising SEQ ID NO: 32. In some embodiments, the recombinant neprosin polypeptide comprising the amino acid sequence comprising residues 105 to 356 of SEQ ID NO: 32 or comprising SEQ ID NO: 32 is glycosylated.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 105 to 356 of SEQ ID NO: 8, or to the reference sequence corresponding to SEQ ID NO: 8.

In some embodiments, the recombinant neprosin polypeptide comprises an acid activated proteolytically active polypeptide derived from or comprising a subsequence of SEQ ID NO: 8. In some embodiments, the recombinant neprosin polypeptide comprises an amino acid sequence comprising residues 105 to 356 of SEQ ID NO: 8. In some embodiments, the recombinant neprosin polypeptide comprises an amino acid sequence comprising SEQ ID NO: 8. In some embodiments, the recombinant neprosin polypeptide comprising the amino acid sequence comprising residues 105 to 356 of SEQ ID NO: 8 or comprising SEQ ID NO: 8 is glycosylated.

In some of the foregoing embodiments, the recombinant neprosin polypeptide is glycosylated. In some embodiments, the recombinant neprosin polypeptide is not glycosylated on at least at amino acid position 44, 121, 128, or 229, wherein the amino acid positions are relative to SEQ ID NO: 8. In some embodiments, the recombinant neprosin polypeptide is glycosylated but is not glycosylated on at least at amino acid position 44, 121, 128, or 229, wherein the amino acid positions are relative to SEQ ID NO: 8.

In some embodiments, the recombinant neprosin polypeptide comprises an amino acid sequence comprising residues 96 to 451 of SEQ ID NO: 58, 60, 62, 64, 66, 68, or 70. In some embodiments, the recombinant neprosin polypeptide comprises a pro-polypeptide of the amino acid sequence comprising amino acid residues 96 to 451 of SEQ ID NO: 58, 60, 62, 64, 66, 68, or 70. In some embodiments, the recombinant neprosin polypeptide comprises a mature polypeptide of the amino acid sequence comprising amino acid residues 96 to 451 of SEQ ID NO: 58, 60, 62, 64, 66, 68, or 70.

In some embodiments, the recombinant neprosin polypeptide further comprises a signal sequence or a signal peptide. In some embodiments, the signal sequence or signal peptide is functional in the host cell used or to be used for expression of the recombinant neprosin polypeptide. In some embodiments the signal sequence or signal peptide is fused to a pro-polypeptide form of the recombinant neprosin, e.g., for forming a pre-pro-polypeptide. In some embodiments, the signal sequence or signal peptide is fused to the polypeptide that includes the mature, active form of the recombinant neprosin. In some embodiments, the signal sequence can be a naturally occurring signal sequence or a synthetic signal sequence, including a hybrid signal sequence.

In some embodiments, the signal sequence or signal peptide is a mammalian or insect cell signal sequence or signal peptide, or a signal sequence or signal peptide functional in mammalian cells or insect cells. Exemplary mammalian or insect signal sequence or signal peptide includes, among others, human OSM, e.g., MGVLLTQRTLLSLVLALLFPSMASM (SEQ ID NO: 78); VSV-G, e.g., MKCLLYLAFLFIGVNC (SEQ ID NO: 79), mouse Ig Kappa, e.g., METDTLLLWVLLLWVPGSTGD (SEQ ID NO: 80), mouse Ig heavy, e.g., MGWSCIILFLVATATGVHS (SEQ ID NO: 81), BM40, e.g., MRAWIFFLLCLAGRALA (SEQ ID NO: 82); Secrecon, e.g., MWWRLWWLLLLLLLLWPMVWA (SEQ ID NO: 83); Human IgKVIII, e.g., MDMRVPAQLLGLLLLWLRGARC (SEQ ID NO: 84); CD33, e.g., MPLLLLLPLLWAGALA (SEQ ID NO: 85); tPA, e.g., MDAMKRGLCCVLLLCGAVFVSPS (SEQ ID NO: 86); human chymotrypsinogen, e.g., MAFLWLLSCWALLGTTFG (SEQ ID NO: 87); Human trypsinogen-2, e.g., MNLLLILTFVAAAVA (SEQ ID NO: 88); human IL-2, e.g., MYRMQLLSCIALSLALVTNS (SEQ ID NO: 89); Gaussia luc, e.g., MGVKVLFALICIAVAEA (SEQ ID NO: 90); human serum albumin (HSA), e.g., MKWVTFISLLFSSAYS (SEQ ID NO: 91); influenza haemagglutinin, e.g., MKTIIALSYIFCLVLG (SEQ ID NO: 92); Human insulin, e.g., MALWMRLLPLLALLALWGPDPAAA (SEQ ID NO: 93); Silkworm Fibroin LC, e.g., MKPIFLVLLVVTSAYA (SEQ ID NO: 94); and honeybee melittin signal peptide of gp64 or gp67, e.g., MLLVNQSHQGFNKEHTSKMVSAIVLYVLLAAAAHSAFA (SEQ ID NO: 95). In some embodiments, the mammalian signal sequence or signal peptide comprises the sequence MEWSWVFLFFLSVTTGVHS (SEQ ID NO: 96).

In some embodiments, the signal sequence or signal peptide is a fungal (e.g., yeast) signal sequence or signal peptide or a signal sequence, or a signal peptide functional in fungal cells. Exemplary fungal signal sequence or signal peptide includes, among others, Pichia pastoris Ost 1, e.g., MRFPSIFTAVLFAASSALAAPVNTTTEDETAQIPAEAVIGYLDLEGDFDVAVLPFSNSTNNGLLFI NTTIASIAAKEEGVSLDKREAEA (SEQ ID NO: 97); Pichia pastoris Pst1, MRFPSIFTAVLFAASSALAAPANTTTFDETAQIPAFAVIDYSDLFGDFDAAALPLSNSTNNGISSTN TTIASIAAKEFGVLEKRFAFA (SEQ ID NO: 98); S. cerevisiae α-mating factor, e.g., MRFPSIFTAVLFAASSALA (SEQ ID NO: 99); S. cerevisiae invertase, e.g., MLLQAFLFLLAGF (SEQ ID NO: 100); Komagataella pastoris yeast α-factor, e.g., MKPLILSAILITLCITSIAVSAPVEGIFADLHDSSHPYITDDVGVRMSAIKEENPDRHFVGDEIPDEA VIQSFKSKRREILFLLDDIGSAVEFIGSNLAQIEANHLSERVQFSWTHIRKNQPFGKREAEA (SEQ ID NO: 101); S. cerevisiae CYP, e.g., MKAFTSLLCGLGLSTTLAKA (SEQ ID NO: 102); Pichia pastoris PH08, e.g., MDSEPLLPNPNDSRKPANWRRIIKYISLTLAWIGIFSYVYIYHGTA (SEQ ID NO: 103); S. cerevisiae PEP4, e.g., MFSLKALLPLALLLVSANQVAA (SEQ ID NO: 104); S. cerevisiae SUC2, e.g., MLLQAFLFLLAGFAAKISA (SEQ ID NO: 105); Pichia pastoris KAR2, e.g., MLSLKPSWLTLAALMYAMLLVVVPFAKPVRA (SEQ ID NO: 106); Pichia pastoris DSE4, e.g., MSFSSNVPQLFLLLVLLTNIVSG (SEQ ID NO: 107); Pichia pastoris EXG1, e.g., MNLYLITLLFASLCSA (SEQ ID NO: 108); and Pichia pastoris SCW10, e.g., MRFSNFLTVSALLTGALG (SEQ ID NO: 109). In some embodiments, the signal sequence or signal peptide comprises the yeast α-mating factor pre-pro sequence comprising residues 1-89 of SEQ ID NO: 54.

Other signal sequences and signal peptides are disclosed in, among others, WO23007468, WO22192675, WO22171827, WO22090555; 052021292776, 0511198881; 0511306127; U.S. Pat. No. 8,377,676; U52010021967; U59273104; and Wu et al., ACS Synth. Biol., 2020, 9(8):2154-2161; all references incorporated by reference herein.

In some embodiments, an existing signal sequence or signal peptide can be replaced with another signal sequence or signal peptide, i.e., a heterologous signal sequence or signal peptide. By way of example and not limitation, the signal sequence of SEQ ID NO: 1 can be replaced with an alternative sequence, such as a signal sequence for expression in a mammalian or insect cell, or a signal sequence for expression in a fungal cell, e.g., Pichia or Saccharomyces. In some embodiments, a signal sequence is operably fused to the pro-polypeptide, or in some embodiments mature polypeptide, of a recombinant neprosin described herein to facilitate expression and/or processing to prepare the recombinant neprosin polypeptide. An exemplary recombinant neprosin polypeptide with a heterologous signal sequence or signal peptide is SEQ ID NO: 38 and SEQ ID NO: 54.

In some embodiments, the recombinant neprosin polypeptide is a fusion protein. In some embodiments, the recombinant neprosin polypeptide described herein can be fused to a variety of polypeptide sequences, such as, by way of example and not limitation, polypeptide tags that can be used for detection and/or purification. In some embodiments, the fusion protein of the recombinant neprosin polypeptide comprises a glycine-histidine or histidine-tag (His-tag). In some embodiments, the fusion protein of the recombinant neprosin polypeptides comprise an epitope tag, such as c-myc, FLAG, V5, or hemagglutinin (HA). In some embodiments, the fusion protein of the recombinant neprosin polypeptide comprises a GST, SUMO, Strep, MBP, or GFP tag. In some embodiments, the fusion is to the amino (N—) terminus of the recombinant neprosin polypeptide. In some embodiments, the fusion is to the carboxy (C-) terminus of the recombinant neprosin polypeptide. In some embodiments, the fusion polypeptide is inserted following the signal sequence and before the neprosin polypeptide to allow expression and secretion of a polypeptide comprising the fusion polypeptide (e.g., polypeptide tag) and recombinant neprosin polypeptide.

In some embodiments, the recombinant neprosin polypeptides includes a signal sequence or signal peptide and/or a polypeptide tag. In some embodiments, the recombinant neprosin polypeptide comprises SEQ ID NO: 36, 40. 42, 44, 46, 48, 50, or 52. In some embodiments, the recombinant neprosin polypeptide comprises SEQ ID NO: 56, 58, 60, 62, 64, 66, 68, or 70.

In some embodiments, the recombinant neprosin polypeptide is a neprosin from Nepenthes species, including, among others, N. x ventrata, N. ampullaria, N. rafflesiana, and N. alata.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 1, 2, 3, 4, 5, or 6.

In some embodiments, the recombinant neprosin polypeptide comprises the sequence corresponding to the pre-pro-polypeptide, pro-polypeptide, mature polypeptide, or polypeptide of SEQ ID NO: 1, 2, 3, 4, 5, or 6. In some embodiments, the recombinant neprosin polypeptide comprises a pro-polypeptide (e.g., pro-enzyme) of the sequence of SEQ ID NO: 1, 2, 3, 4, 5, or 6. In some embodiments, the recombinant neprosin polypeptide comprises a mature polypeptide of the sequence of SEQ ID NO: 1, 2, 3, 4, 5, or 6. In some embodiments, the recombinant neprosin polypeptide comprises an acid activated, proteolytically active polypeptide derived from or comprising a subsequence of the sequence corresponding to SEQ ID NO: 1, 2, 3, 4, 5, or 6.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 1.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 1.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 1.

In some embodiments, the recombinant neprosin polypeptide comprises an amino acid sequence comprising the pre-pro-polypeptide of SEQ ID NO: 1. In some embodiments, the recombinant neprosin polypeptide comprises an amino acid sequence comprising the pro-polypeptide of SEQ ID NO: 1. In some embodiments, the recombinant neprosin polypeptide comprises an amino acid sequence comprising the mature polypeptide of SEQ ID NO: 1. In some embodiments, the recombinant neprosin polypeptide comprises an acid activated, proteolytically active polypeptide derived from or comprising a subsequence of the sequence corresponding to SEQ ID NO: 1. In some embodiments, the mature neprosin polypeptide of SEQ ID NO: 1 has a molecular weight of about 25.5 kDa to about 29 kDa, particularly as determined by SDS-PAGE.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 2.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 2.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 2.

In some embodiments, the recombinant neprosin polypeptide comprises an amino acid sequence comprising the pre-pro-polypeptide of SEQ ID NO: 2. In some embodiments, the recombinant neprosin polypeptide comprises an amino acid sequence comprising the pro-polypeptide of SEQ ID NO: 2. In some embodiments, the recombinant neprosin polypeptide comprises an amino acid sequence comprising the mature polypeptide of SEQ ID NO: 2. In some embodiments, the recombinant neprosin polypeptide comprises an acid activated, proteolytically active polypeptide derived from or comprising a subsequence of the sequence corresponding to SEQ ID NO: 2.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 3.

In some embodiments, the recombinant neprosin polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to the pro-polypeptide of SEQ ID NO: 3.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 3.

In some embodiments, the recombinant neprosin polypeptide comprises an amino acid sequence comprising the pre-pro-polypeptide of SEQ ID NO: 3. In some embodiments, the recombinant neprosin polypeptide comprises an amino acid sequence comprising the pro-polypeptide of SEQ ID NO: 3. In some embodiments, the recombinant neprosin polypeptide comprises an amino acid sequence comprising the mature polypeptide of SEQ ID NO: 3. In some embodiments, the recombinant neprosin polypeptide comprises an acid activated, proteolytically active polypeptide derived from or comprising a subsequence of the sequence corresponding to SEQ ID NO: 3.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 4.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 4.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 4.

In some embodiments, the recombinant neprosin polypeptide comprises an amino acid sequence comprising the pre-pro-polypeptide of SEQ ID NO: 4. In some embodiments, the recombinant neprosin polypeptide comprises an amino acid sequence comprising the pro-polypeptide of SEQ ID NO: 4. In some embodiments, the recombinant neprosin polypeptide comprises an amino acid sequence comprising the mature polypeptide of SEQ ID NO: 4. In some embodiments, the recombinant neprosin polypeptide comprises an acid activated, proteolytically active polypeptide derived from or comprising a subsequence of the sequence corresponding to SEQ ID NO: 4.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 5.

In some embodiments, the recombinant neprosin polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to the pro-polypeptide of SEQ ID NO: 5.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the mature polypeptide of SEQ ID NO: 5.

In some embodiments, the recombinant neprosin polypeptide comprises an amino acid sequence comprising the pre-pro-polypeptide of SEQ ID NO: 5. In some embodiments, the recombinant neprosin polypeptide comprises an amino acid sequence comprising the pro-polypeptide of SEQ ID NO: 5. In some embodiments, the recombinant neprosin polypeptide comprises an amino acid sequence comprising the mature polypeptide of SEQ ID NO: 5. In some embodiments, the recombinant neprosin polypeptide comprises an acid activated, proteolytically active polypeptide derived from or comprising a subsequence of the sequence corresponding to SEQ ID NO: 5.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 6.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the pro-polypeptide of SEQ ID NO: 6.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the mature polypeptide of SEQ ID NO: 6.

In some embodiments, the recombinant neprosin polypeptide comprises an amino acid sequence comprising the pre-pro-polypeptide of SEQ ID NO: 6. In some embodiments, the recombinant neprosin polypeptide comprises an amino acid sequence comprising the pro-polypeptide of SEQ ID NO: 6. In some embodiments, the recombinant neprosin polypeptide comprises an amino acid sequence comprising the mature polypeptide of SEQ ID NO: 6. In some embodiments, the recombinant neprosin polypeptide comprises an acid activated, proteolytically active polypeptide derived from or comprising a subsequence of the sequence corresponding to SEQ ID NO: 6.

Recombinant Polynucleotides, Expression Vectors, and Host Cells

In a further aspect, the present disclosure provides a recombinant polynucleotide encoding a recombinant neprosin polypeptide described herein, as well as expression vectors for expression of the recombinant neprosin polypeptide in appropriate host cells.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode a recombinant neprosin polypeptide of the present disclosure. Thus, the present disclosure provides methods and compositions for the production of each and every possible variation of recombinant neprosin polynucleotides that could be made that encode the recombinant neprosin polypeptides described herein by selecting combinations based on the possible codon choices, and all such variations are to be considered specifically disclosed for any polypeptide described herein, including the amino acid sequences of recombinant neprosin polypeptides described herein.

In some embodiments, the codons are preferably optimized for utilization by the chosen host cell for protein production. In some embodiments, the polynucleotide encoding the recombinant neprosin polypeptide preferably uses codons used in mammalian cells, insect cells, or fungal cells. In some embodiments, codon optimized polynucleotides encoding a recombinant neprosin polypeptide described herein contain preferred codons at about 40%, 50%, 60%, 70%, 80%, 90%, or greater than 90% of the codon positions in the coding region.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to the pre-pro-polypeptide, pro-polypeptide, mature polypeptide, or polypeptide of SEQ ID NO: 1, wherein the amino acid sequence comprises at least at amino acid position 68, 145, 152, or 253, or combinations thereof, and equivalent positions thereof, a substitution with an amino acid other than asparagine (N), wherein the amino acid positions are relative to the reference sequence corresponding to SEQ ID NO: 1. In some embodiments, the amino acid substitution or amino acid residue at position 68, 145, 152, or 253 is independently selected from A, C, D, E, F, G, H, I, K, L, M, P, Q, R, S, T, V, W, and Y. In some embodiments, the amino acid substitution or amino acid residue at position 68, 145, 152, or 253 is each A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 68, or equivalent position thereof, a substitution with an amino acid other than asparagine (N), wherein the amino acid position is relative to the reference sequence corresponding to SEQ ID NO: 1. In some embodiments, the amino acid substitution or amino acid residue at position 68 is selected from A, C, D, E, F, G, H, I, K, L, M, P, Q, R, S, T, V, W, and Y. In some embodiments, the amino acid substitution or amino acid residue at position 68 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 145, or equivalent position thereof, a substitution with an amino acid other than asparagine (N), wherein the amino acid position is relative to SEQ ID NO: 1. In some embodiments, the amino acid substitution or amino acid residue at position 145 is selected from A, C, D, E, F, G, H, I, K, L, M, P, Q, R, S, T, V, W, and Y. In some embodiments, the amino acid substitution or amino acid residue at position 145 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at amino acid position 152, or equivalent position thereof, a substitution with an amino acid other than asparagine (N), wherein the amino acid position is relative to SEQ ID NO: 1. In some embodiments, the amino acid substitution or amino acid residue at position 152 is selected from A, C, D, E, F, G, H, I, K, L, M, P, Q, R, S, T, V, W, and Y. In some embodiments, the amino acid substitution or amino acid residue at position 152 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid position 253, or equivalent position thereof, a substitution with an amino acid other than asparagine (N), wherein the amino acid position is relative to SEQ ID NO: 1. In some embodiments, the amino acid substitution or amino acid residue at position 253 is selected from A, C, D, E, F, G, H, I, K, L, M, P, Q, R, S, T, V, W, and Y. In some embodiments, the amino acid substitution or amino acid residue at position 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 145, or equivalent positions thereof, a substitution with an amino acid other than asparagine (N), wherein the amino acid positions are relative to SEQ ID NO: 1. In some embodiments, the amino acid substitution or amino acid residue at each of positions 68 and 145 is independently selected from A, C, D, E, F, G, H, I, K, L, M, P, Q, R, S, T, V, W, and Y. In some embodiments, the amino acid substitution or amino acid residue at positions 68 and 145 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 152, or equivalent positions thereof, a substitution with an amino acid other than asparagine (N), wherein the amino acid positions are relative to SEQ ID NO: 1. In some embodiments, the amino acid substitution or amino acid residue at each of positions 68 and 152 is independently selected from A, C, D, E, F, G, H, I, K, L, M, P, Q, R, S, T, V, W, and Y. In some embodiments, the amino acid substitution or amino acid residue at positions 68 and 152 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68 and 253 is independently selected from A, C, D, E, F, G, H, I, K, L, M, P, Q, R, S, T, V, W, and Y. In some embodiments, the amino acid substitution or amino acid residue at positions 68 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 145 and 152, or equivalent positions thereof, a substitution with an amino acid other than asparagine (N), wherein the amino acid positions are relative to SEQ ID NO: 1. In some embodiments, the amino acid substitution or amino acid residue at each of positions 145 and 152 is independently selected from A, C, D, E, F, G, H, I, K, L, M, P, Q, R, S, T, V, W, and Y. In some embodiments, the amino acid substitution or amino acid residue at positions 145 and 152 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid at positions 145 and 253, or equivalent positions thereof, a substitution with an amino acid other than asparagine (N), wherein the amino acid positions are relative to SEQ ID NO: 1. In some embodiments, the amino acid substitution or amino acid residue at each of positions 145 and 253 is independently selected from A, C, D, E, F, G, H, I, K, L, M, P, Q, R, S, T, V, W, and Y. In some embodiments, the amino acid substitution or amino acid residue at positions 145 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 152 and 253 is independently selected from A, C, D, E, F, G, H, I, K, L, M, P, Q, R, S, T, V, W, and Y. In some embodiments, the amino acid substitution or amino acid residue at positions 152 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145 and 152, or equivalent positions thereof, a substitution with an amino acid other than asparagine (N), wherein the amino acid positions are relative to SEQ ID NO: 1. In some embodiments, the amino acid substitution or amino acid residue at each of positions 68, 145 and 152 is independently selected from A, C, D, E, F, G, H, I, K, L, M, P, Q, R, S, T, V, W, and Y. In some embodiments, the amino acid substitution or amino acid residue at positions 68, 145 and 152 is

A.

In some embodiments, the recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 145 and 253 is independently selected from A, C, D, E, F, G, H, I, K, L, M, P, Q, R, S, T, V, W, and Y. In some embodiments, the amino acid substitution or amino acid residue at positions 68, 145 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 152, and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 152, and 253 is independently selected from A, C, D, E, F, G, H, I, K, L, M, P, Q, R, S, T, V, W, and Y. In some embodiments, the amino acid substitution or amino acid residue at positions 68, 152, and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 145, 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 145, 152 and 253 is independently selected from A, C, D, E, F, G, H, I, K, L, M, P, Q, R, S, T, V, W, and Y. In some embodiments, the amino acid substitution or amino acid residue at positions 145, 152 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least at amino acid positions 68, 145, 152 and 253, or equivalent positions thereof, a substitution with an amino acid other than asparagine (N), wherein the amino acid positions are relative to SEQ ID NO: 1. In some embodiments, the amino acid substitution or amino acid residue at each of positions 68, 145, 152 and 253 is independently selected from A, C, D, E, F, G, H, I, K, L, M, P, Q, R, S, T, V, W, and Y. In some embodiments, the amino acid substitution or amino acid residue at positions 68, 145, 152 and 253 is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N68A, N145A, N152A, or N253A, or equivalent positions thereof, and combinations thereof, wherein the amino acid positions are relative to SEQ ID NO: 1.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to the pre-pro-polypeptide, pro-polypeptide, mature polypeptide, or polypeptide of SEQ ID NO: 1, wherein the amino acid sequence comprises at least a substitution N68A, or equivalent position thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N145A, or equivalent position thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N152A, or equivalent position thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least a substitution N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A and N152A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N145A and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N152A and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A, N152A, and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N145A, N152A, and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to the pre-pro-polypeptide, pro-polypeptide, mature polypeptide, or polypeptide of SEQ ID NO: 1, wherein the amino acid sequence comprises at least substitutions N68A, N145A, N152A, and N253A, or equivalent positions thereof.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the polypeptide sequence comprising residues 26 to 381 of SEQ ID NO: 44, or a pro-polypeptide or mature polypeptide thereof, wherein the amino acid at position 68, 152, and/or 254 is other than an asparagine (N).

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the polypeptide sequence comprising residues 26 to 381 of SEQ ID NO: 46, or a pro-polypeptide or mature polypeptide thereof, wherein the amino acid at position 68 is other than an asparagine (N).

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the polypeptide sequence comprising residues 26 to 381 of SEQ ID NO: 48, or a pro-polypeptide or mature polypeptide thereof, wherein the amino acid at position 152 is other than an asparagine (N).

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the polypeptide sequence comprising residues 26 to 381 of SEQ ID NO: 50, or a pro-polypeptide or mature polypeptide thereof, wherein the amino acid at position 254 is other than an asparagine (N).

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the polypeptide sequence comprising residues 26 to 381 of SEQ ID NO: 52, or a pro-polypeptide or mature polypeptide thereof, wherein the amino acid at position 68 and 254 is other than an asparagine (N). In some embodiments, the amino acid at positions 68 and 254 is alanine (A).

In some embodiments, the recombinant polynucleotide comprises a polynucleotide sequence encoding a recombinant neprosin polypeptide comprising an amino acid sequence comprising residues 26 to 381 of SEQ ID NO: 44, 46, 48, 50, or 52. In some embodiments, the recombinant polynucleotide comprises a polynucleotide sequence encoding a recombinant neprosin polypeptide comprising a pro-polypeptide of the polypeptide sequence comprising amino acid residues 26 to 381 of SEQ ID NO: 44, 46, 48, 50, or 52. In some embodiments, the recombinant polynucleotide comprises a polynucleotide sequence encoding a recombinant neprosin polypeptide comprising a mature polypeptide of the polypeptide sequence comprising amino acid residues 26 to 381 of SEQ ID NO: 44, 46, 48, 50, or 52.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the recombinant polynucleotide comprises a polynucleotide sequence encoding a recombinant neprosin polypeptide comprising an amino acid sequence comprising residues 105 to 356 of SEQ ID NO: 8, wherein the amino acid sequence comprises at least at amino acid position 121, 128, or 229, or combinations thereof, a substitution with an amino acid other than asparagine (N), wherein the amino acid positions are relative to SEQ ID NO: 8. In some embodiments, the encoded recombinant neprosin polypeptide comprises an amino acid sequence comprising SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 8, or to the reference sequence corresponding to SEQ ID NO: 8, wherein the amino acid sequence comprises at least at amino acid position 44, a substitution with an amino acid other than asparagine (N), wherein the amino acid position is relative to SEQ ID NO: 8. In some embodiments, the amino acid substitution or amino acid residue at position 44 is selected from A, C, D, E, F, G, H, I, K, L, M, P, Q, R, S, T, V, W, and Y. In some embodiments, the amino acid substitution or amino acid residue at position 44 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 10. In some embodiments, the encoded recombinant neprosin polypeptide comprises an amino acid sequence comprising residues 105 to 356 of SEQ ID NO: 10. In some embodiments, the encoded recombinant neprosin polypeptide comprises an amino acid sequence comprising SEQ ID NO: 10.

In some embodiments, the recombinant polynucleotide comprising a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 121 a substitution with an amino acid other than asparagine (N), wherein the amino acid position is relative to SEQ ID NO: 8. In some embodiments, the amino acid substitution or amino acid residue at position 121 is selected from A, C, D, E, F, G, H, I, K, L, M, P, Q, R, S, T, V, W, and Y. In some embodiments, the amino acid substitution or amino acid residue at position 121 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 12. In some embodiments, the encoded recombinant neprosin polypeptide comprises an amino acid sequence comprising residues 105 to 356 of SEQ ID NO: 12. In some embodiments, the encoded recombinant neprosin polypeptide comprises an amino acid sequence comprising SEQ ID NO: 12.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 128 a substitution with an amino acid other than asparagine (N), wherein the amino acid position is relative to SEQ ID NO: 8. In some embodiments, the amino acid substitution or amino acid residue at position 128 is selected from A, C, D, E, F, G, H, I, K, L, M, P, Q, R, S, T, V, W, and Y. In some embodiments, the amino acid substitution or amino acid residue at position 128 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 14. In some embodiments, the encoded recombinant neprosin polypeptide comprises an amino acid sequence comprising residues 105 to 356 of SEQ ID NO: 14. In some embodiments, the encoded recombinant neprosin polypeptide comprises an amino acid sequence comprising SEQ ID NO: 14.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 229 a substitution with an amino acid other than asparagine (N), wherein the amino acid position is relative to SEQ ID NO: 8. In some embodiments, the amino acid substitution or amino acid residue at position 229 is selected from A, C, D, E, F, G, H, I, K, L, M, P, Q, R, S, T, V, W, and Y. In some embodiments, the amino acid substitution or amino acid residue at position 229 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 16. In some embodiments, the encoded recombinant neprosin polypeptide comprises an amino acid sequence comprising residues 105 to 356 of SEQ ID NO: 16. In some embodiments, the encoded recombinant neprosin polypeptide comprises an amino acid sequence comprising SEQ ID NO: 16.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 121 and 229 substitutions with an amino acid other than asparagine (N), wherein the amino acid positions are relative to SEQ ID NO: 8. In some embodiments, the amino acid substitutions or amino acid residues at each of positions 121 and 229 is independently selected from A, C, D, E, F, G, H, I, K, L, M, P, Q, R, S, T, V, W, and Y. In some embodiments, the amino acid substitution or amino acid residue at amino acid positions 121 and 229 is A.

In some embodiments, the recombinant polynucleotide comprises a polynucleotide sequence encoding a recombinant neprosin polypeptide comprising an acid activated, proteolytically active polypeptide derived from or comprising a subsequence of SEQ ID NO: 18. In some embodiments, the encoded recombinant neprosin polypeptide comprises an amino acid sequence comprising residues 105 to 356 of SEQ ID NO: 18. In some embodiments, the encoded recombinant neprosin polypeptide comprises an amino acid sequence comprising SEQ ID NO: 18.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 128, and 229 substitutions with an amino acid other than asparagine (N), wherein the amino acid positions are relative to SEQ ID NO: 8. In some embodiments, the amino acid substitutions or amino acid residues at each of positions 44, 128, and 229 is independently selected from A, C, D, E, F, G, H, I, K, L, M, P, Q, R, S, T, V, W, and Y. In some embodiments, the amino acid substitution or amino acid residue at amino acid positions 44, 128, and 229 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 20. In some embodiments, the encoded recombinant neprosin polypeptide comprises an amino acid sequence comprising residues 105 to 356 of SEQ ID NO: 20. In some embodiments, the encoded recombinant neprosin polypeptide comprises an amino acid sequence comprising SEQ ID NO: 20.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 121, 128, and 229 substitutions with an amino acid other than asparagine (N), wherein the amino acid positions are relative to SEQ ID NO: 8. In some embodiments, the amino acid substitution or amino acid residue at each of positions 44, 121, 128, and 229 is independently selected from A, C, D, E, F, G, H, I, K, L, M, P, Q, R, S, T, V, W, and Y. In some embodiments, the amino acid substitution or amino acid residue at amino acid positions 44, 121, 128, and 229 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 22. In some embodiments, the encoded recombinant neprosin polypeptide comprises an amino acid sequence comprising residues 105 to 356 of SEQ ID NO: 22. In some embodiments, the encoded recombinant neprosin polypeptide comprises an amino acid sequence comprising SEQ ID NO: 22.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44 and 229 substitutions with an amino acid other than asparagine (N), wherein the amino acid position is relative to SEQ ID NO: 8. In some embodiments, the amino acid substitution or amino acid residue at each of positions 44 and 229 is independently selected from A, C, D, E, F, G, H, I, K, L, M, P, Q, R, S, T, V, W, and Y. In some embodiments, the amino acid substitution or amino acid residue at amino acid positions 44 and 229 is A.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 32. In some embodiments, the encoded recombinant neprosin polypeptide comprises an amino acid sequence comprising residues 105 to 356 of SEQ ID NO: 32. In some embodiments, the encoded recombinant neprosin polypeptide comprises an amino acid sequence comprising SEQ ID NO: 32.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to residues 105 to 356 of SEQ ID NO: 8, or to the sequence corresponding to SEQ ID NO: 8.

In some embodiments, the recombinant polynucleotide comprises a polynucleotide sequence encoding a recombinant neprosin polypeptide comprising an acid activated proteolytically active polypeptide derived from or comprising a subsequence of SEQ ID NO: 8. In some embodiments, the encoded recombinant neprosin polypeptide comprises an amino acid sequence comprising residues 105 to 356 of SEQ ID NO: 8. In some embodiments, the encoded recombinant neprosin polypeptide comprises an amino acid sequence comprising SEQ ID NO: 8.

In some embodiments, the recombinant polynucleotide comprises a polynucleotide sequence encoding a recombinant neprosin polypeptide comprising an amino acid sequence comprising residues 96 to 451 of SEQ ID NO: 58, 60, 62, 64, 66, 68, or 70. In some embodiments, the recombinant polynucleotide comprises a polynucleotide sequence encoding a recombinant neprosin polypeptide comprising a pro-polypeptide of the amino acid sequence comprising amino acid residues 96 to 451 of SEQ ID NO: 58, 60, 62, 64, 66, 68, or 70. In some embodiments, the recombinant polynucleotide comprises a polynucleotide sequence encoding a recombinant neprosin polypeptide comprising a mature polypeptide of the amino acid sequence comprising amino acid residues 96 to 451 of SEQ ID NO: 58, 60, 62, 64, 66, 68, or 70.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 313 to 1068 of SEQ ID NO: 7, 9, 11, 13, 15, 17, 19, 21, 23, 25, 27, 29, 31, or 33, or a polynucleotide sequence corresponding to SEQ ID NO: 7, 9, 11, 13, 15, 17, 19, 21, 23, 25, 27, 29, 31, or 33, wherein the recombinant polynucleotide encodes a neprosin polypeptide.

In some embodiments, the recombinant polynucleotide comprises a polynucleotide sequence comprising nucleotide residues 313 to 1068 of SEQ ID NO: 7, 9, 11, 13, 15, 17, 19, 21, 23, 25, 27, 29, 31, or 33, or comprising SEQ ID NO: 7, 9, 11, 13, 15, 17, 19, 21, 23, 25, 27, 29, 31, or 33.

In some embodiments, the recombinant polynucleotide comprises a polynucleotide sequence encoding a recombinant neprosin polypeptide comprising a signal sequence or a signal peptide, as discussed herein. In some embodiments, the encoded signal sequence or signal peptide is functional in the host cell used or to be used for expression of the neprosin polypeptide. In some embodiments the encoded signal sequence or signal peptide is fused to a pro-polypeptide form of the recombinant neprosin for forming a pre-pro-polypeptide. In some embodiments, the encoded signal sequence or signal peptide is fused to the polypeptide that includes the mature, active form of the recombinant neprosin. In some embodiments, the encoded signal sequence can be a naturally occurring signal sequence or a synthetic signal sequence, including a hybrid signal sequence.

In some embodiments, the recombinant polynucleotide comprises a polynucleotide sequence encoding a recombinant neprosin polypeptide comprising a fusion protein. In some embodiments, the encoded recombinant neprosin polypeptide can be fused to a variety of polypeptide sequences as described above. In some embodiments, the fusion protein of the recombinant neprosin polypeptides comprises a glycine-histidine or histidine-tag (His-tag). In some embodiments, the fusion protein of the recombinant neprosin polypeptides comprise an epitope tag, such as c-myc, FLAG, V5, or hemagglutinin (HA). In some embodiments, the fusion protein of the recombinant neprosin polypeptides comprises a GST, SUMO, Strep, MBP, or GFP tag.

In some embodiments, the recombinant polynucleotide comprises a polynucleotide sequence encoding a recombinant neprosin polypeptides which includes a signal sequence and/or signal peptide, and a polypeptide tag. In some embodiments, the recombinant polynucleotide comprises a polynucleotide sequence encoding a recombinant neprosin polypeptide comprising SEQ ID NO: 36, 38, 40, 42, 44, 46, 48, 50, or 52. In some embodiments, the recombinant polynucleotide comprises a polynucleotide sequence encoding a recombinant neprosin polypeptide comprising SEQ ID NO: 56, 58, 60, 62, 64, 66, 68, or 70.

In some embodiments, the recombinant polynucleotide comprises a polynucleotide sequence comprising SEQ ID NO: 35, 37, 39, 41, 43, 45, 47, 49, or 51. In some embodiments, the recombinant polynucleotide comprises a polynucleotide sequence encoding a recombinant neprosin polypeptide comprising SEQ ID NO: 55, 57, 59, 61, 63, 65, 67, or 69.

In some embodiments, the recombinant polynucleotide comprises a polynucleotide sequence encoding a recombinant neprosin polypeptide comprising SEQ ID NO:

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 1, 2, 3, 4, 5, or 6.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to the pre-pro-polypeptide, pro-polypeptide, mature polypeptide, or polypeptide of SEQ ID NO: 1.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 1.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO:

1.

In some embodiments, the recombinant polynucleotide comprises a polynucleotide sequence encoding a recombinant neprosin polypeptide comprising an amino acid sequence comprising the pre-pro-polypeptide of SEQ ID NO: 1. In some embodiments, the recombinant polynucleotide comprising a polynucleotide sequence encoding a recombinant neprosin polypeptide comprising an amino acid sequence comprising the pro-polypeptide of SEQ ID NO: 1. In some embodiments, the recombinant polynucleotide comprises a polynucleotide sequence encoding a recombinant neprosin polypeptide comprising an amino acid sequence comprising the mature polypeptide of SEQ ID NO: 1. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 1.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 2.

In some embodiments, the recombinant polynucleotide comprises a polynucleotide sequence encoding a recombinant neprosin polypeptide comprising an amino acid sequence having at least 80%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 2.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO:

2.

In some embodiments, the recombinant polynucleotide comprises a polynucleotide sequence encoding a recombinant neprosin polypeptide comprising an amino acid sequence comprising the pre-pro-polypeptide of SEQ ID NO: 2. In some embodiments, the recombinant polynucleotide comprising a polynucleotide sequence encoding a recombinant neprosin polypeptide comprising an amino acid sequence comprising the pro-polypeptide of SEQ ID NO: 2. In some embodiments, the recombinant polynucleotide comprises a polynucleotide sequence encoding a recombinant neprosin polypeptide comprising an amino acid sequence comprising the mature polypeptide of SEQ ID NO: 2. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 2.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 3.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 3.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 3.

In some embodiments, the recombinant polynucleotide comprises a polynucleotide sequence encoding a recombinant neprosin polypeptide comprising an amino acid sequence comprising the pre-pro-polypeptide of SEQ ID NO: 3. In some embodiments, the recombinant polynucleotide comprises a polynucleotide sequence encoding a recombinant neprosin polypeptide comprising an amino acid sequence comprising the pro-polypeptide of SEQ ID NO: 3. In some embodiments, the recombinant polynucleotide comprises a polynucleotide sequence encoding a recombinant neprosin polypeptide comprising an amino acid sequence comprising the mature polypeptide of SEQ ID NO: 3. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 3.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 4.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 4.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the mature polypeptide of SEQ ID NO: 4.

In some embodiments, the recombinant polynucleotide comprises a polynucleotide sequence encoding a recombinant neprosin polypeptide comprising an amino acid sequence comprising the pre-pro-polypeptide of SEQ ID NO: 4. In some embodiments, the recombinant polynucleotide comprises a polynucleotide sequence encoding a recombinant neprosin polypeptide comprising an amino acid sequence comprising the pro-polypeptide of SEQ ID NO: 4. In some embodiments, the recombinant polynucleotide comprises a polynucleotide sequence encoding a recombinant neprosin polypeptide comprising an amino acid sequence comprising the mature polypeptide of SEQ ID NO: 4. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 4.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 5.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 5.

In some embodiments, the recombinant polynucleotide comprises a polynucleotide sequence encoding a recombinant neprosin polypeptide comprising an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to the mature polypeptide of SEQ ID NO: 5.

In some embodiments, the recombinant polynucleotide comprises a polynucleotide sequence encoding a recombinant neprosin polypeptide comprising an amino acid sequence comprising the pre-pro-polypeptide of SEQ ID NO: 5. In some embodiments, the recombinant polynucleotide comprises a polynucleotide sequence encoding a recombinant neprosin polypeptide comprising an amino acid sequence comprising the pro-polypeptide of SEQ ID NO: 5. In some embodiments, the recombinant polynucleotide comprises a polynucleotide sequence encoding a recombinant neprosin polypeptide comprising an amino acid sequence comprising the mature polypeptide of SEQ ID NO: 5. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 5.

In some embodiments, the recombinant polynucleotide encodes a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pre-pro-polypeptide, pro-polypeptide, mature polypeptide, or polypeptide of SEQ ID NO: 6.

In some embodiments, the recombinant polynucleotide comprises a polynucleotide sequence encoding a recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to the pro-polypeptide of SEQ ID NO: 6.

In some embodiments, the recombinant polynucleotide comprises a polynucleotide sequence encoding a recombinant neprosin polypeptide comprising an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to the mature polypeptide of SEQ ID NO: 6.

In some embodiments, the recombinant polynucleotide comprises a polynucleotide sequence encoding a recombinant neprosin polypeptide comprising an amino acid sequence comprising the pre-pro-polypeptide of SEQ ID NO: 6. In some embodiments, the recombinant polynucleotide comprises a polynucleotide sequence encoding a recombinant neprosin polypeptide comprising an amino acid sequence comprising the pro-polypeptide of SEQ ID NO: 6. In some embodiments, the recombinant polynucleotide comprises a polynucleotide sequence encoding a recombinant neprosin polypeptide comprising an amino acid sequence comprising the mature polypeptide of SEQ ID NO: 6. In some embodiments, the recombinant polynucleotide comprises a polynucleotide sequence encoding a recombinant neprosin polypeptide comprising an acid activated, proteolytically active polypeptide derived from or comprising a subsequence of the sequence corresponding to SEQ ID NO: 6.

In another aspect, the present disclosure further provides an expression vector comprising a recombinant polynucleotide encoding a recombinant neprosin polypeptide described herein, e.g., for expression of the encoded recombinant neprosin polypeptides. In some embodiments, the expression vector comprises one or more control sequences to regulate the expression of the neprosin polynucleotides and/or polypeptide. In some embodiments, the control sequences include among others, promoters, leader sequences, polyadenylation sequences, pro-peptide sequences, signal peptide sequences, and transcription terminators. In particular, the control sequences such as promoters, leader sequences, polyadenylation sequences, pro-peptide sequences, signal peptide sequences, and transcription terminators are those effective in a chosen host cell, for example mammalian cells, insect cells, or fungal cells.

In some embodiments, suitable promoters for directing transcription of the nucleic acid constructs in mammalian cells, include, among others, SV40 promoters or a promoter from cytomegalovirus (CMV), chicken beta-actin (CAG), or the elongation factor (EF)-1. In some embodiments, suitable promoters for directing transcription of the nucleic acid constructs in insect cells, include, among others, baculovirus promoters (e.g., P10 and polyhedron promoters), OpIE2 promoter, and Nephotettix cincticeps actin promoters. In some embodiments, suitable promoters for directing transcription in fungal cells include, among others, promoters of AOX1 gene, TEF1 gene, PDX2 gene, TDH3 gene, PGK gene, ADH1 gene, as well as hybrid promoters (see, e.g., Mojzita et al., Curr Opin Biotech., 2019, 59:141-149; Yang et al., World Journal of Microbiology and Biotechnology, 2015, 31:1641-1646). Other fungal promoters include promoters of genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase.

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the neprosin polypeptide. Any suitable terminator which is functional in the host cell of choice finds use in for the expression of neprosin polypeptide. By way of example and not limitation, transcription terminators include those from human β-globin gene, SV40, hGH, IE1 terminator, and BGH. Exemplary fungal transcription terminators include, among others, those from genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the recombinant neprosin polypeptide. Any suitable leader sequence that is functional in the host cell of choice find use in expression of the recombinant neprosin polypeptide. Exemplary leader sequences for mammalian and insect cells include, among others, leaders sequences of expressed genes (e.g., heat shock protein, myosin, BIP immunoglobulin binding protein, GRP glucose regulated protein, etc.), viral leader sequences (e.g., EMC virus) and synthetic leaders sequences, e.g., hTEE-658 and those described in, for example Cao et al., Nature Commun., 2021, 12:4138, incorporated herein by reference. Exemplary leader sequences for fungal expression include, among others, those from Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for mammalian and insect cells include, among others, those of genes for human and mouse alpha-globin, mouse kappa light chain, chicken ovalbumin, SV40, as wells a synthetic polyA sequences (see, e.g., Clerici et al., eLife, 2017, 6:e33111). Exemplary polyadenylation sequences for fungal host cells include, but not limited to, the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known in the art (see, e.g., Guo and Sherman, Mol. Cell. Bio., 1995, 15:5983-5990).

In some embodiments, the control sequence is also a signal peptide (i.e., a coding region that codes for an amino acid sequence linked to the amino terminus of a polypeptide and directs the encoded polypeptide into the cell's secretory pathway), as described herein. In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence, i.e., a heterologous signal sequence. Any suitable signal peptide coding region which directs the expressed polypeptide into the secretory pathway of a host cell of choice finds use for expression of the engineered polypeptide(s). In some embodiments, the signal sequence is a mammalian cell signal sequence. In some embodiments, the signal sequence is an insect cell signal sequence. In some embodiments, the signal sequence is a fungal cell signal sequence. Exemplary signal sequences are described herein.

In another aspect, the present disclosure provides an expression vector comprising a recombinant polynucleotide encoding a recombinant neprosin polypeptide, and one or more expression regulating regions such as a promoter and a terminator, a replication origin, etc., depending on the type of hosts into which they are to be introduced. In some embodiments, the various nucleic acid and control sequences described herein are joined together (i.e., operably linked) to produce recombinant expression vectors which include one or more convenient restriction sites to allow for insertion or substitution of the nucleic acid sequence encoding the recombinant neprosin polypeptide at such sites.

The recombinant expression vector may be any suitable vector (e.g., a plasmid or virus), that can be conveniently subjected to recombinant DNA procedures and bring about the expression of the neprosin-encoding polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Suitable markers for mammalian cells, depending on cell type, include, among others, adenosine deaminase (ADA), aminoglycoside phosphotransferase (neo, G418, APH), bleomycin (Sh ble), cytosine deaminase, dihydrofolate reductase (DHFR), histidinol dehydrogenase (hisD), hygromycin-B-phosphotransferase (HPH), thymidine kinase (TK), and xanthine-guanine phosphoribosyltransferase (XGPRT, gpt). Suitable markers for insect cells, depending on cell type, include, among others, puromycin acetyltransferase, aminoglycoside phosphotransferase, and hygromycin-B-phosphotransferase (hygromycin resistance). Suitable markers for fungal host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In a further aspect, the present disclosure provides a host cell comprising an expression vector described herein, wherein the host cell is a eukaryotic cell. In some embodiments, the host cell is a mammalian cell, an insect cell, or a fungal cell.

In some embodiments, the host cell expresses a recombinant neprosin polypeptide described herein. In some embodiments, the recombinant neprosin polypeptide expressed in the host cell is a pre-pro-polypeptide or pre-pro-enzyme form of neprosin polypeptide. In some embodiments, the recombinant neprosin polypeptide expressed in the host cell is a pro-polypeptide or pro-enzyme form of neprosin polypeptide. In some embodiments, the recombinant neprosin polypeptide expressed in the host cell is a mature neprosin polypeptide.

In some embodiments, host cell comprises a polynucleotide sequence encoding a recombinant neprosin polypeptide, wherein the encoded recombinant neprosin polypeptide includes a signal sequence. In some embodiments, the signal sequence is selected as appropriate for the host cell. In some embodiments, the signal sequence is a mouse signal sequence. In some embodiments, the signal sequence is a human signal sequence. In some embodiments, the signal sequence is an insect cell signal sequence. In some embodiments, the signal sequence is a fungal signal sequence. In some embodiments, the host cell expresses a recombinant neprosin polypeptide which is secreted. In some embodiments, the host cell expresses recombinant neprosin polypeptide in soluble form.

In some embodiments, the host cell expresses a recombinant neprosin polypeptide which is glycosylated. In some embodiments, the host cell expresses a recombinant neprosin polypeptide which is glycosylated but lack glycosylation on at least at one of the glycosylation sites, as described herein. In some embodiments, the host cell expresses a recombinant neprosin polypeptide which is non-glycosylated. In some embodiments, the glycosylation pattern of a recombinant neprosin polypeptide is manipulated by suitable selection of a host cell.

In some embodiments, the host cell is a mammalian cell. In some embodiments, the host cell is a human cell or rodent cell. Exemplary mammalian cells include, among others, Expi293, HeLa, U2OS, A549, HT1080, CAD, P19, NIH 3T3, L929, Hek 293, 293F, 293E, 293T, COS, Vero, NSO, Sp2/0 cell, DUKX-X11, MCF-7, Y79, SO-Rb50, Hep G2, J558L, and CHO cell. In some embodiments, the mammalian host cell is defective in glycosylation (see, e.g., Esko et al., Glycosylation Mutants of Cultured Mammalian Cells. 2017. In: Varki A, Cummings R D, Esko J D, et al., editors. Essentials of Glycobiology, 3rd Ed., Chapter 49, Cold Spring Harbor (NY): Cold Spring Harbor Laboratory Press; 2015-2017). In some embodiments, the polynucleotide sequence encoding the recombinant neprosin polypeptide in the host mammalian cell is codon optimized for expression in mammalian cells.

In some embodiments, the host cell is an insect cell. In some embodiments, the insect host cell is a lepidopteran or dipteran insect cell. Exemplary insect host cell includes, among others, Sf9 cell, Sf21 cell, Schneider 2 cell, and BTI-TN-5B1-4 (High Five) cell. In some embodiments, the insect host cell contains humanized glycosylation pathway enzymes (see, e.g., Yee et al., Ind. Eng. Chem. Res., 2018, 57:10061-10070). In some embodiments, the polynucleotide sequence encoding the recombinant neprosin polypeptide in the host insect cell is codon optimized for expression in insect cells.

In some embodiments, the host cell is a fungal cell. In some embodiments, the fungal cell is a filamentous fungal cell or fungi. In some embodiments, the fungal cell is a yeast cell. In some embodiments, the fungal host cell is a Pichia, Saccharomyces, Yarrowia, Kluyveromyces, Aspergillus, Trichoderma, Neurospora, Mucor, Penicillium T. Trichoderma, or Myceliophthora fungal cell. Exemplary fungal host cell includes, among others, Pichia pastoris, Yarrowia lipolytica, Kluyveromyces marxianus, Kluyveromyces lactis, Aspergillus niger, Aspergillus oryzae, Aspergillus fumigatus Trichoderma reesei. Neurospora crassa, Mucor circinelloides, Penicillium chrysogenum T. reesei, Trichoderma harzianum, Saccharomyces cerevisiae, or Myceliophthora thermophile. In some embodiments, the polynucleotide sequence encoding the recombinant neprosin polypeptide in the host fungal cell is codon optimized for expression in fungal cells. In preferred embodiments, the fungal cell for expression of the recombinant neprosin polypeptide is Pichia pastoris or Saccharomyces cerevisiae.

In some embodiments, any suitable method for introducing polynucleotides for expression of the recombinant neprosin polypeptides into cells will find use for the purposes herein. Suitable techniques include, but are not limited to electroporation, biolistic particle bombardment, liposome mediated transfection, calcium chloride transfection, and protoplast fusion.

Expression and Processing of Recombinant Neprosin Polypeptide

In another aspect, the present disclosure provides a method of expressing a recombinant neprosin polypeptide described herein. In some embodiments, the present disclosure further provides processing of the expressed recombinant neprosin polypeptide to prepare proteolytically active recombinant neprosin polypeptide, particularly proteolytically active form of mature neprosin polypeptide.

In some embodiments, a method of producing a recombinant neprosin polypeptide comprises culturing a host cell comprising an expression vector comprising a recombinant polynucleotide encoding a recombinant neprosin polypeptide described herein under suitable culture conditions such that the encoded recombinant neprosin polypeptide is expressed or produced. In some embodiments, the host cell is a mammalian cell, insect cell, or fungal cell.

In some embodiments, the method of producing a recombinant neprosin polypeptide comprises culturing a mammalian cell comprising an expression vector comprising a recombinant polynucleotide encoding a recombinant neprosin polypeptide described herein under suitable culture conditions such that the encoded recombinant neprosin polypeptide is expressed or produced.

As described herein, in some embodiments, the mammalian cell is a rodent cell or human cell. Exemplary mammalian cell for use as a host cell include Expi293, HeLa, U205, A549, HT1080, CAD, P19, NIH 3T3, L929, Hek 293, 293F, 293E, 293T, COS, Vero, NS0 cell, Sp2/0, DUKX-X11, MCF-7, Y79, SO-Rb50, Hep G2, J558L, and CHO cell. In some embodiments, the mammalian cell is defective in glycosylation (see, e.g., Esko et al., Glycosylation Mutants of Cultured Mammalian Cells. 2017. In: Varki A, Cummings R D, Esko J D, et al., editors. Essentials of Glycobiology, 3rd Ed., Chapter 49, Cold Spring Harbor (NY): Cold Spring Harbor Laboratory Press; 2015-2017).

In some embodiments, the method of producing a recombinant neprosin polypeptide comprises culturing an insect cell comprising an expression vector comprising a recombinant polynucleotide encoding a recombinant neprosin polypeptide described herein under suitable culture conditions such that the encoded recombinant neprosin polypeptide is expressed or produced.

As described above, in some embodiments, the insect cell is a lepidopteran or dipteran insect cell. Exemplary insect cells for use as a host cell include Sf9 cell, Sf21 cell, BTI-TN-5B1-4 (High Five) cell, and Schneider 2 cell. In some embodiments, the insect cell contains humanized glycosylation pathway enzymes (see, e.g., Yee et al., Ind. Eng. Chem. Res., 2018, 57:10061-10070).

In some embodiments, the method of producing a recombinant neprosin polypeptide comprises culturing a fungal cell comprising an expression vector comprising a recombinant polynucleotide encoding a recombinant neprosin polypeptide described herein under suitable culture conditions such that the encoded recombinant neprosin polypeptide is expressed or produced.

As described above, in some embodiments, the fungal cell is a filamentous fungal cell or fungi. In some embodiments, the fungal cell is a yeast cell. Exemplary fungal cell for use as a host cell include Aspergillus, Arxula, Aurantiochytrium, Candida, Claviceps, Cryptococcus, Cunninghamella, Geotrichum, Hansemula, Kluyveromyces, Kodamaea, Komagataella, Leucosporidiella, Lipomyces, Morterella, Mucor, Myceliophthora, Neurospora, Ogataea, Penicillium, Pichia, Prototheca, Rhizopus, Rhodosporidium, Rhodotorula, Saccharomyces, Tremella, Trichoderma, and Yarrowia fungal cell. In some embodiments, the fungal cell for use as a host cell is Pichia pastoris, Yarrowia lipolytica, Kluyveromyces marxianus, Kluyveromyces lactis, Aspergillus niger, Aspergillus oryzae, Aspergillus fumigatus Trichoderma reesei. Neurospora crassa, Mucor circinelloides, Penicillium chrysogenum T. reesei, Trichoderma harzianum, Saccharomyces cerevisiae, and Myceliophthora thermophile.

Appropriate culture media and growth conditions for mammalian, insect cells, and fungal cells for expressing heterologous proteins are well known in the art. Exemplary culture medium for mammalian cells, include, among others, Eagle's Minimum Essential Medium (EMEM), Dulbecco's Modified Eagle's Medium (DMEM), Iscove's Modified Dulbecco's Medium (IMDM), Hybri-Care Medium, McCoy's 5A and RPMI-1640, Ham's Nutrient Mixtures, and DMEM/F12 Medium. Such media formulations are described in, among others, ATCC Animal Cell Culture Guide, 2022; Phelan, K. and May, K. M., 2017, “Mammalian cell tissue culture techniques,” Curr. Protoc. Mol. Biol. 117:A.3F.1-A.3F.23.; and Freshney, IF, “Culture of Animal Cells: A Manual of Basic Technique and Specialized Applications,” 7th Ed., Wiley-Blackwell (2016).

Exemplary culture medium for insect cells, include, among others, Grace's Insect Medium, Complete TNM-FH, Sf 900 II Serum Free Medium, Express Five Serum Free Medium, Mitsuhashi and Maramorosh Basal Medium, HH70 medium, and Schneider's Drosophila medium. Such media formulations are described in, among others, Agathos, S. N. “Development of serum-free media for lepidopteran insect cell lines,” In Baculovirus Expression Protocols, 2nd Ed., D. W. Murhammer, ed., p. 155-185, Humana Press, Totowa, N J (2007); and KaBer et al., Electronic Journal of Biotechnology, 2022, 56:54-64.

Culture medium for fungal cells may vary depending on the type of fungal cell used for expression of the recombinant neprosin polypeptide. Exemplary medium for Pichia pastoris is Rich defined medium (RDM) or Buffered complex glycerol medium (BMGY) (see Mathews et al., Biotechnol Bioeng, 2018, 115(1):103-113); YNB/YND medium (yeast nitrogen base), YPM medium, or BMM (buffered minimal medium) (see, e.g., Lin-Cereghino et al., “Expression of protein in Pichia pastoris,” In Expression systems: methods express, Dyson M, Durocher Y (ed), p 123-145, Scion Publishing, Banbury, United Kingdom (2007). Exemplary culture medium for S. cerevisiae include, among others, YPD and YEPD medium (see, e.g., Dymond, J S., Methods Enzymol., 2013, 533:191-204).

In some embodiments, the method further comprises isolating the expressed recombinant neprosin polypeptide. In some embodiments, the method further comprises the step of purifying the expressed recombinant neprosin polypeptide. In some embodiments, the isolated or purified neprosin polypeptide comprises the pre-pro-polypeptide (e.g., pre-pro-enzyme) form of the recombinant neprosin polypeptide. In some embodiments, the isolated or purified neprosin polypeptide comprises the pro-polypeptide (e.g., pro-enzyme) form of the recombinant neprosin polypeptide. In some embodiments, the isolated or purified neprosin polypeptide comprises the mature form of the recombinant neprosin polypeptide.

In some embodiments, the recombinant neprosin polypeptide expressed or produced in a host cell are isolated and/or purified from the cells and/or the culture medium using any one or more of the known techniques for protein purification, including, among others, detergent treatment, sonication, filtration, salting-out, ultra-centrifugation, and chromatography. Chromatographic techniques for isolation and purification of the neprosin polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular enzyme depends, in part, on factors such as net charge, hydrophobicity, hydrophilicity, molecular weight, molecular shape, etc., and will be apparent to those having skill in the art. In some embodiments, affinity techniques may be used to isolate the recombinant neprosin polypeptides. For affinity chromatography purification, an antibody that specifically binds neprosin polypeptide of interest may find use. In some embodiments, the recombinant neprosin polypeptide is isolated and/or purified using a polypeptide tag present on the protein, for example a His-tag.

In some embodiments, the method is used to express a pre-pro-polypeptide (e.g., pre-pro-enzyme) form of a recombinant or naturally occurring neprosin polypeptide. In some embodiments, the method is used to express a pro-polypeptide, e.g., pro-enzyme, form of the recombinant or naturally occurring neprosin polypeptide. In some embodiments, the method is used to express a mature polypeptide form of the recombinant or naturally occurring neprosin polypeptide.

In some embodiments, the method is used to express a recombinant neprosin polypeptide which is secreted. In some embodiments, the recombinant neprosin polypeptide is expressed in soluble form. In some embodiments, the secreted form is the pro-polypeptide form of a recombinant neprosin polypeptide.

In some embodiments, the method is used to express recombinant neprosin polypeptide which is glycosylated. In some embodiments, the method is used to express pre-pro-polypeptide of a recombinant neprosin polypeptide which is glycosylated. In some embodiments, the method is used to express a pro-polypeptide of a recombinant neprosin polypeptide which is glycosylated. In some embodiments, the method is used to express a mature polypeptide of a recombinant neprosin polypeptide which is glycosylated. In some embodiments, the expressed recombinant neprosin polypeptide is glycosylated at one or more positions 68, 145, 152, or 253, or combinations thereof, wherein the amino acid positions are relative to SEQ ID NO: 1. In some embodiments, the expressed recombinant neprosin polypeptide is glycosylated at one or more positions 44, 121, 128, or 229, or combinations thereof, wherein the amino acid positions are relative to SEQ ID NO: 8.

In some embodiments, targeting of glycosylation can use a recombinant polynucleotide encoding a recombinant neprosin polypeptide having a mutation at amino acid position 68, 145, 152, or 253, or combinations thereof, wherein the amino acid positions are relative to SEQ ID NO: 1, to express recombinant neprosin polypeptide glycosylated at defined positions. In some embodiments, the expressed recombinant neprosin polypeptide is glycosylated at least at amino acid position 68. In some embodiments, the expressed recombinant neprosin polypeptide is glycosylated at least at amino acid position 145. In some embodiments, the expressed recombinant neprosin polypeptide is glycosylated at least at amino acid position 152. In some embodiments, the expressed recombinant neprosin polypeptide is glycosylated at least at amino acid position 253.

In some embodiments, the expressed recombinant neprosin polypeptide is non-glycosylated. In some embodiments, the expressed recombinant neprosin is a pre-pro-polypeptide which is non-glycosylated. In some embodiments, the expressed recombinant neprosin is a pro-polypeptide which is non-glycosylated. In some embodiments, the expressed recombinant neprosin is a mature polypeptide which is non-glycosylated.

As discussed herein, the method is used to express a recombinant neprosin polypeptide, including pre-pro-polypeptide, pro-polypeptide, or mature forms of the recombinant neprosin polypeptide, in mammalian cells, insect cells, or fungal cells. In some preferred embodiments, the method is used to express a recombinant neprosin polypeptide in fungal cells, particularly Pichia pastoris.

In some embodiments, the expressed recombinant neprosin polypeptide lacks glycosylation at one or more of position 68, 145, 152, and 253, or combinations thereof, wherein the amino acid positions are relative to SEQ ID NO: 1. In some embodiments, the expressed recombinant neprosin polypeptide lacks glycosylation at least at position 68 or equivalent position thereof. In some embodiments, the expressed recombinant neprosin polypeptide lacks glycosylation at least at position 145, or equivalent position thereof. In some embodiments, the expressed recombinant neprosin polypeptide lacks glycosylation at least at position 152, or equivalent position thereof. In some embodiments, the expressed recombinant neprosin polypeptide lacks glycosylation at least at position 253, or equivalent position thereof. In some embodiments, the expressed recombinant neprosin polypeptide lacks glycosylation at least at positions 68 and 152, or equivalent positions thereof. In some embodiments, the expressed recombinant neprosin polypeptide lacks glycosylation at least at positions 68 and 253, or equivalent positions thereof. In some embodiments, the expressed recombinant neprosin polypeptide lacks glycosylation at least at positions 152 and 253, or equivalent positions thereof.

In some embodiments, the expressed recombinant neprosin polypeptide lacks glycosylation at one or more of position 44, 121, 128, or 229, or combinations thereof, wherein the amino acid positions are relative to SEQ ID NO: 8. In some embodiments, the expressed recombinant neprosin polypeptide lacks glycosylation at least at position 44 or equivalent position thereof. In some embodiments, the expressed recombinant neprosin polypeptide lacks glycosylation at least at position 121, or equivalent position thereof. In some embodiments, the expressed recombinant neprosin polypeptide lacks glycosylation at least at position 128, or equivalent position thereof. In some embodiments, the expressed recombinant neprosin polypeptide lacks glycosylation at least at position 229, or equivalent position thereof. In some embodiments, the expressed recombinant neprosin polypeptide lacks glycosylation at least at positions 44 and 128, or equivalent positions thereof. In some embodiments, the expressed recombinant neprosin polypeptide lacks glycosylation at least at positions 44 and 229, or equivalent positions thereof. In some embodiments, the expressed recombinant neprosin polypeptide lacks glycosylation at least at positions 128 and 229, or equivalent positions thereof. In some embodiments, the expressed recombinant neprosin polypeptide lacks glycosylation at least at positions 12 and 229, or equivalent positions thereof.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the expressed recombinant neprosin polypeptide comprises an amino acid sequence comprising residues 105 to 356 of SEQ ID NO: 8, wherein the amino acid sequence comprises at least at amino acid position 121, 128, or 229, or combinations thereof, a substitution with an amino acid other than asparagine (N), wherein the amino acid positions are relative to SEQ ID NO: 8. In some embodiments, the recombinant neprosin polypeptide comprises an amino acid sequence comprising SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8. In some embodiments, the amino acid substitution or amino acid residue at amino acid position 44, 121, 128, or 229, or combinations thereof, is independently selected from A, C, D, E, F, G, H, I, K, L, M, P, Q, R, S, T, V, W, and Y. In some embodiments, the amino acid substitution or amino acid residue at position 44, 121, 128, or 229, or combinations thereof, is A.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 44 a substitution with an amino acid other than asparagine (N), wherein the amino acid position is relative to SEQ ID NO: 8. In some embodiments, the amino acid substitution or amino acid residue at position 44 is selected from A, C, D, E, F, G, H, I, K, L, M, P, Q, R, S, T, V, W, and Y. In some embodiments, the amino acid substitution or amino acid residue at position 44 is A.

In some embodiments, the expressed recombinant neprosin polypeptide comprises an acid activated proteolytically active polypeptide derived from or comprising a subsequence of SEQ ID NO: 10. In some embodiments, the expressed recombinant neprosin polypeptide comprises an amino acid sequence comprising residues 105 to 356 of SEQ ID NO: 10. In some embodiments, the expressed recombinant neprosin polypeptide comprises an amino acid sequence comprising SEQ ID NO: 10. In some embodiments, the expressed recombinant neprosin polypeptide comprising the amino acid sequence comprising residues 105 to 356 of SEQ ID NO: 10 or comprising SEQ ID NO: 10 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 121 a substitution with an amino acid other than asparagine (N), wherein the amino acid position is relative to SEQ ID NO: 8. In some embodiments, the amino acid substitution or amino acid residue at position 121 is selected from A, C, D, E, F, G, H, I, K, L, M, P, Q, R, S, T, V, W, and Y. In some embodiments, the amino acid substitution or amino acid residue at position 121 is A.

In some embodiments, the expressed recombinant neprosin polypeptide comprises an acid activated proteolytically active polypeptide derived from or comprising a subsequence of SEQ ID NO: 12. In some embodiments, the expressed recombinant neprosin polypeptide comprises an amino acid sequence comprising residues 105 to 356 of SEQ ID NO: 12. In some embodiments, the expressed recombinant neprosin polypeptide comprises an amino acid sequence comprising SEQ ID NO: 12. In some embodiments, the expressed recombinant neprosin polypeptide comprising the amino acid sequence comprising residues 105 to 356 of SEQ ID NO: 12 or comprising SEQ ID NO: 12 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid position 128 a substitution with an amino acid other than asparagine (N), wherein the amino acid position is relative to SEQ ID NO: 8. In some embodiments, the amino acid substitution or amino acid residue at position 128 is selected from A, C, D, E, F, G, H, I, K, L, M, P, Q, R, S, T, V, W, and Y. In some embodiments, the amino acid substitution or amino acid residue at position 128 is A.

In some embodiments, the expressed recombinant neprosin polypeptide comprises an acid activated proteolytically active polypeptide derived from or comprising a subsequence of SEQ ID NO: 14. In some embodiments, the expressed recombinant neprosin polypeptide comprises an amino acid sequence comprising residues 105 to 356 of SEQ ID NO: 14. In some embodiments, the expressed recombinant neprosin polypeptide comprises an amino acid sequence comprising SEQ ID NO: 14. In some embodiments, the expressed recombinant neprosin polypeptide comprising the amino acid sequence comprising residues 105 to 356 of SEQ ID NO: 14 or comprising SEQ ID NO: 14 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to residues 105 to 356 of SEQ ID NO: 8, or to the sequence corresponding to SEQ ID NO: 8, wherein the amino acid sequence comprises at least at amino acid position 229, or equivalent position thereof, a substitution with an amino acid other than asparagine (N), wherein the amino acid position is relative to SEQ ID NO: 8. In some embodiments, the amino acid substitution or amino acid residue at position 229 is selected from A, C, D, E, F, G, H, I, K, L, M, P, Q, R, S, T, V, W, and Y. In some embodiments, the amino acid substitution or amino acid residue at position 229 is A.

In some embodiments, the expressed recombinant neprosin polypeptide comprises an acid activated proteolytically active polypeptide derived from or comprising a subsequence of SEQ ID NO: 16. In some embodiments, the expressed recombinant neprosin polypeptide comprises an amino acid sequence comprising residues 105 to 356 of SEQ ID NO: 16. In some embodiments, the expressed recombinant neprosin polypeptide comprises an amino acid sequence comprising SEQ ID NO: 16. In some embodiments, the recombinant neprosin polypeptide comprising the amino acid sequence comprising residues 105 to 356 of SEQ ID NO: 12 or comprising SEQ ID NO: 16 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 121 and 229 substitutions with an amino acid other than asparagine (N), wherein the amino acid positions are relative to SEQ ID NO: 8. In some embodiments, the amino acid substitution or amino acid residue at each of positions 121 and 229 is independently selected from A, C, D, E, F, G, H, I, K, L, M, P, Q, R, S, T, V, W, and Y. In some embodiments, the amino acid substitution or amino acid residue at positions 121 and 229 is A.

In some embodiments, the expressed recombinant neprosin polypeptide comprises an acid activated, proteolytically active polypeptide derived from or comprising a subsequence of SEQ ID NO: 18. In some embodiments, the expressed recombinant neprosin polypeptide comprises an amino acid sequence comprising residues 105 to 356 of SEQ ID NO: 18. In some embodiments, the expressed recombinant neprosin polypeptide comprises an amino acid sequence comprising SEQ ID NO: 18. In some embodiments, the recombinant neprosin polypeptide comprising the amino acid sequence comprising residues 105 to 356 of SEQ ID NO: 18 or comprising SEQ ID NO: 18 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 128, and 229 substitutions with an amino acid other than asparagine (N), wherein the amino acid position is relative to SEQ ID NO: 8. In some embodiments, the amino acid substitution or amino acid residue at each of positions 44, 128, and 229 is independently selected from A, C, D, E, F, G, H, I, K, L, M, P, Q, R, S, T, V, W, and Y. In some embodiments, the amino acid substitution or amino acid residue at positions 44, 128, and 229 is A.

In some embodiments, the expressed recombinant neprosin polypeptide comprises an acid activated proteolytically active polypeptide derived from or comprising a subsequence of SEQ ID NO: 20. In some embodiments, the expressed recombinant neprosin polypeptide comprises an amino acid sequence comprising residues 105 to 356 of SEQ ID NO: 20. In some embodiments, the recombinant neprosin polypeptide comprises an amino acid sequence comprising SEQ ID NO: 20. In some embodiments, the recombinant neprosin polypeptide comprising the amino acid sequence comprising residues 105 to 356 of SEQ ID NO: 20 or comprising SEQ ID NO: 20 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44, 121, 128, and 229 substitutions with an amino acid other than asparagine (N), wherein the amino acid positions are relative to SEQ ID NO: 8. In some embodiments, the amino acid substitution or amino acid residue at each of positions 44, 121, 128, and 229 is independently selected from A, C, D, E, F, G, H, I, K, L, M, P, Q, R, S, T, V, W, and Y. In some embodiments, the amino acid substitution or amino acid residue at positions 44, 121, 128, and 229 is A.

In some embodiments, the expressed recombinant neprosin polypeptide comprises an acid activated proteolytically active polypeptide derived from or comprising a subsequence of SEQ ID NO: 22. In some embodiments, the expressed recombinant neprosin polypeptide comprises an amino acid sequence comprising residues 105 to 356 of SEQ ID NO: 22. In some embodiments, the expressed recombinant neprosin polypeptide comprises an amino acid sequence comprising SEQ ID NO: 22. In some embodiments, the expressed recombinant neprosin polypeptide comprising the amino acid sequence comprising residues 105 to 356 of SEQ ID NO: 22 or comprising SEQ ID NO: 22 is not glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 105 to 356 of SEQ ID NO: 8, or to the reference sequence corresponding to SEQ ID NO: 8, wherein the amino acid sequence comprises at least at amino acid positions 44 and 229 substitution with an amino acid other than asparagine (N), wherein the amino acid positions are relative to SEQ ID NO: 8. In some embodiments, the amino acid substitution or amino acid residue at each of positions 44 and 229 is independently selected from A, C, D, E, F, G, H, I, K, L, M, P, Q, R, S, T, V, W, and Y. In some embodiments, the amino acid substitution or amino acid residue at positions 44 and 229 is A.

In some embodiments, the expressed recombinant neprosin polypeptide comprises an acid activated proteolytically active polypeptide derived from or comprising a subsequence of SEQ ID NO: 32. In some embodiments, the expressed recombinant neprosin polypeptide comprises an amino acid sequence comprising residues 105 to 356 of SEQ ID NO: 32. In some embodiments, the expressed recombinant neprosin polypeptide comprises an amino acid sequence comprising SEQ ID NO: 32. In some embodiments, the expressed recombinant neprosin polypeptide comprising the amino acid sequence comprising residues 105 to 356 of SEQ ID NO: 32 or comprising SEQ ID NO: 32 is glycosylated.

In some embodiments, the expressed recombinant neprosin polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the sequence corresponding to residues 105 to 356 of SEQ ID NO: 8, or to the sequence corresponding to SEQ ID NO: 8.

In some embodiments, the expressed recombinant neprosin polypeptide comprises an acid activated proteolytically active polypeptide derived from or comprising a subsequence of SEQ ID NO: 8. In some embodiments, the expressed recombinant neprosin polypeptide comprises an amino acid sequence comprising residues 105 to 356 of SEQ ID NO: 8. In some embodiments, the expressed recombinant neprosin polypeptide comprises an amino acid sequence comprising SEQ ID NO: 8. In some embodiments, the expressed recombinant neprosin polypeptide comprising the amino acid sequence residues 105 to 356 of SEQ ID NO: 8 or comprising SEQ ID NO: 22 is glycosylated.

In some embodiments, the expressed recombinant neprosin polypeptide comprises an amino acid sequence comprising residues 26 to 381 of SEQ ID NO: 44, 46, 48, 50, or 52. In some embodiments, the expressed recombinant neprosin polypeptide comprises a pro-polypeptide contained in residues 26 to 381 of SEQ ID NO: 44, 46, 48, 50, or 52. In some embodiments, the expressed recombinant neprosin polypeptide comprises a mature neprosin polypeptide contained in residues 26 to 381 of SEQ ID NO: 44, 46, 48, 50, or 52. In some embodiments, the expressed recombinant neprosin polypeptide comprises an amino acid sequence comprising SEQ ID NO: 36, 38, 40, 42, 44, 46, 48, 50, or 52.

In some embodiments, the expressed recombinant neprosin polypeptide comprises an amino acid sequence comprising residues 96 to 451 of SEQ ID NO: 58, 60, 62, 64, 66, 68, or 70. In some embodiments, the expressed recombinant neprosin polypeptide comprises a pro-polypeptide contained in residues 96 to 451 of SEQ ID NO: 58, 60, 62, 64, 66, 68, or 70. In some embodiments, the expressed recombinant neprosin polypeptide comprises a mature neprosin polypeptide contained in residues 96 to 451 of SEQ ID NO: 58, 60, 62, 64, 66, 68, or 70. In some embodiments, the expressed recombinant neprosin polypeptide comprises an amino acid sequence comprising SEQ ID NO: 54, 56, 58, 60, 62, 64, 66, 68, or 70.

In some embodiments, a method of preparing a mature neprosin polypeptide comprises treating a full-length neprosin polypeptide or a neprosin pre-pro-polypeptide or pro-polypeptide prepared from expressed neprosin described herein; or treating a full-length neprosin polypeptide or a neprosin pre-pro-polypeptide or pro-polypeptide of the recombinant neprosin variants described herein, to an acidic pH under suitable conditions for formation of the mature neprosin polypeptide. In some embodiments, the treating is at a pH of about 2 to about 6. In some embodiments, the treating is at a pH of about 2 to about 4.5. In some embodiments, the treating is at a pH of about 2 to about 3. In some embodiments, the method of preparing the mature neprosin polypeptide is carried out in vitro. In some embodiments, the mature neprosin polypeptide produced by the method has a molecular weight of about 25.5 kDa to about 29 kDa, particularly as determined by SDS-PAGE.

In some embodiments, the pro-peptide is removed at acidic pH, thereby activating the enzyme and forming a mature neprosin polypeptide. In some embodiments the pro-peptide is cleaved by a cellular protease, thereby activating the enzyme and forming a mature neprosin polypeptide. In some embodiments, the cell expresses a mature form of the neprosin polypeptide.

In some embodiments, the present disclosure provides mature, proteolytically active recombinant neprosin polypeptide prepared by acid activation of a pre-pro-polypeptide or a pro-polypeptide of a recombinant neprosin polypeptide described herein.

Pharmaceutical and Dietary/Nutritional Compositions

In a further aspect, the recombinant neprosin is formulated as a composition. In some embodiments, the composition comprises a recombinant pre-pro-polypeptide or pre-pro-enzyme form of a neprosin polypeptide, a pro-polypeptide or pro-enzyme form of a neprosin polypeptide, or a mature neprosin polypeptide, as described herein. In some embodiments, the recombinant neprosin polypeptide in the composition is prepared from expressed recombinant neprosin polypeptide or a recombinant neprosin polypeptide variant, including pre-pro-polypeptide, pro-polypeptide, or mature polypeptide forms for the variants as described herein. In some embodiments, the mature neprosin polypeptide in the composition has a molecular weight of about 25.5 kDa to about 29 kDa, for example as determined by SDS-PAGE. In some embodiments, the recombinant neprosin polypeptide in the composition is prepared from recombinant neprosin polypeptide expressed or produced in mammalian cells, insect cells, or fungal cells.

In some embodiments, the recombinant neprosin is formulated as a pharmaceutical composition, dietary/nutritional supplements, or in food compositions.

In some embodiments, the recombinant neprosin polypeptide may be used in any suitable edible enzyme delivery matrix. In some embodiments, recombinant neprosin polypeptide are present in an edible enzyme delivery matrix designed for rapid dispersal of the neprosin within the digestive tract of an animal or subject upon ingestion of the polypeptide.

In some embodiments, the recombinant neprosin is formulated as a pharmaceutical or dietary composition. Depending on the mode of administration, the compositions comprise a therapeutically effective amount of a recombinant neprosin polypeptide and can be in the form of a solid, semi-solid, or liquid. The term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers.

In some embodiments, the present disclosure provides a pharmaceutical composition comprising a recombinant neprosin polypeptide, and a pharmaceutically acceptable carrier, excipient, or diluent. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, incorporated in its entirety by reference herein. Such compositions will contain a therapeutically effective amount of the enzyme(s), preferably in purified form, together with a suitable amount of carrier so as to provide the form for proper administration to the subject. The formulation should suit the mode of administration.

In some embodiments, the recombinant neprosin polypeptide is formulated for use as oral pharmaceutical compositions (e.g., oral administration). Any suitable format for use in delivering the neprosin polypeptide may be used, including but not limited to pills, tablets, gel tabs, capsules, lozenges, dragees, powders, soft gels, sol-gels, gels, emulsions, implants, patches, sprays, ointments, liniments, creams, pastes, jellies, paints, aerosols, chewing gums, demulcents, sticks, suspensions (including but not limited to oil-based suspensions, oil-in water emulsions, etc.), slurries, syrups, controlled release formulations, suppositories, etc. For oral administration, the pharmaceutical composition can be used alone or in combination with appropriate additives to make the tablets, powders, granules, capsules, syrups, liquids, suspensions, etc. For example, solid oral forms of the composition can be prepared with conventional additives, disintegrators, lubricants, diluents, buffering agents, moistening agents, preservatives and flavoring agents. Non-limiting examples of excipients include sugars (e.g., lactose, sucrose, mannitol, and/or sorbitol), starches (e.g., corn, wheat, rice, potato, or other plant starch), cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.). Additional components in oral formulations may include coloring and or sweetening agents (e.g., glucose, sucrose, and mannitol) and lubricating agents (e.g., magnesium stearate), as well as enteric coatings (e.g., methacrylate polymers, hydroxyl propyl methyl cellulose phthalate, and/or any other suitable enteric coating known in the art). In some embodiments, the formulation releases the enzyme(s) in the stomach of the subject so that the peptidic food antigen(s) can be degraded by the enzyme(s).

In some embodiments, the recombinant neprosin polypeptide is provided as a unit dose formulation. For example, and without limitation, the unit dose may be present in a tablet, a capsule, and the like. The unit dose may be in solid, liquid, powder, or any other form. A unit dose formulation of the pharmaceutical composition will allow for proper dosing (e.g., based on the amount of immunogenic protein ingested) while avoiding potential negative side effects of administering an excessive amount of the composition.

In some embodiments, the recombinant neprosin polypeptide or composition thereof, including as a pharmaceutical composition, can be lyophilized from an aqueous solution, optionally in the presence of appropriate buffers (e.g., phosphate, citrate, histidine, imidazole buffers) and excipients (e.g., cryoprotectants such as sucrose, lactose, trehalose). Lyophilized cakes can optionally be blended with excipients and made into different forms.

Without being bound by theory, it is believed that neprosin may be less active or substantially inactive at neutral to basic pH. This can be important where there is a potential for undesirable digestion by the enzyme(s). For example, where the pharmaceutical composition is administered orally, buffering of the composition to pH 6.5 or greater may result in a less active form of the enzyme(s) such that the oral mucosa, esophageal mucosa, and other cells that may come into contact with the composition will not be digested by the enzyme(s) therein. Likewise, when the composition is added to a food, the buffered enzyme(s) will be unable to (or less able to) digest the food before it is consumed. In such situations, introduction of the composition to the acidic environment of the stomach may result in a decrease in the pH and activation of enzyme(s).

Accordingly, in some embodiments, the pharmaceutical composition is buffered to about pH 6.5 or higher. In a preferred embodiment, the composition is buffered to about pH 6.5 to about pH 8.5. In some embodiments, the composition is in liquid form. In some embodiments, the composition is in semi-solid form. In some embodiments, the composition is in solid form. In some embodiments, the pH of the composition is adjusted in liquid form and the composition is dried to form a solid. In some embodiment, the enzyme(s) in the composition is activated upon contact with acid (i.e., in the stomach).

In some embodiments, the pharmaceutical composition comprising a recombinant neprosin polypeptide is formulated in a delayed release vehicle such that the enzyme(s) is released continuously while the formulation is present in the stomach. In some embodiment, the formulation has a pH of greater than about 5 prior to contact with acids in the stomach. In some embodiments, the formulation comprises a biologically acceptable buffer, such that the pH of the composition remains at about pH 5 or 6 for at least a period of time upon contact with acids in the stomach. In a preferred embodiment, the formulation is a controlled release formulation. The term “controlled release formulation” includes sustained release and time-release formulations. Controlled release formulations are well-known in the art. These include excipients that allow for sustained, periodic, pulse, or delayed release of the drug. Controlled release formulations include, without limitation, embedding of the drug into a matrix; enteric coatings; micro-encapsulation; gels and hydrogels; and any other formulation that allows for controlled release of a drug.

It is also to be understood that in some embodiments, a proteolytically active neprosin polypeptide can be used in the composition, for example the proteolytically active mature neprosin polypeptide. In some embodiments, the proteolytically active mature neprosin polypeptide in the composition is treated to an acidic pH to prepare the active mature polypeptide. In some embodiments, the proteolytically active neprosin polypeptide can be used to treat food prior to ingestion or orally administered concurrently with ingestion of food.

In some embodiments, the composition is a food product comprising a recombinant neprosin polypeptide. In some embodiments, the food is a liquid, semi-solid, or a solid. In some embodiments, the food product comprises gluten or is suspected of comprising gluten, such as bakery products (e.g., cakes, muffins, donuts, pastries, rolls, and bread), pasta, crackers, tortilla chips, cereal etc. made from wheat, rye and barley. In some embodiments, the food product can be consumed with another food product comprising gluten or suspected of comprising gluten. Non-limiting examples of such food include a powder, a spread, a spray, a sauce, a dip, a whipped cream, candies, chewing gums, syrup, sugar, salt, salad dressing, spices, cheese, butter, margarines, spreads, butter, frying shortenings, mayonnaises, dairy products, nut butters, seed butters, kernel butters, peanut butter, etc.

In some embodiments, the recombinant neprosin polypeptide or composition thereof is admixed with food, or used to pre-treat foodstuffs containing glutens. The recombinant neprosin polypeptide in foods can be enzymatically active to reduce the level of gluten in the food prior to or during ingestion. In some embodiments, the recombinant neprosin polypeptide is dispersed into a food prior to consumption, optionally at a pH where it is inactive, such as a pH of about or above 5. Preferably, the food items or additives comprising the neprosin polypeptide do not require heating before being ingested by a patient so that possible loss of activity of the enzyme(s) due to elevated temperature can be minimized.

In some embodiments, a dispenser comprising an inner excipient and an effective amount of the recombinant neprosin polypeptide or composition thereof is used to dispense neprosin or composition thereof to digest gluten in food. In some embodiments, the recombinant neprosin polypeptide or composition thereof and/or inner excipient are added to food before the food is consumed. In some embodiments, the inner excipient comprises sodium chloride or sodium iodide, or a mixture thereof. In some embodiments, the pharmaceutical composition and/or inner excipient are in granular form, sized to efficiently dispense from said dispenser.

In some embodiments, the recombinant neprosin polypeptide or composition thereof (e.g., such as a pharmaceutical composition, edible composition, or composition with additional proteases) or food product comprises from about 0.1% to about 99%, from about 0.5% to about 95%, from about 1% to about 95%, from about 5% to about 95%, from about 10% to about 90%, from about 20% to about 80%, from about 25% to about 75% of neprosin polypeptide. In some embodiments, the amount of recombinant neprosin polypeptide in the composition (such as pharmaceutical composition or edible composition) or food product is about 0.01%, about 0.1%, about 0.5%, about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, or about 95% of the total composition or food product, or a range between any two of the values (including end points).

In some embodiments, the composition, including a pharmaceutical composition comprises a recombinant neprosin polypeptide and one or more additional proteases. In some embodiments, the one or more additional protease is an aspartic protease, a serine protease, a threonine protease, a cysteine protease, a glutamic acid protease, or a metalloprotease. In some embodiments, the additional protease is a nepenthes protease, such as nepenthesin I and/or nepenthesin II, as described herein. In some embodiments, the composition comprises one or more additional exoproteases, such as, leucine aminopeptidases and carboxypeptidases. In some embodiment, the one or more additional protease is trypsin. In a preferred embodiment, the one or more additional protease is active at acidic pH (e.g., pH 2-6). Exemplary proteases that can be useful in the present invention include, without limitation, nepenthesin I, nepenthesin II, BACE, cathepsin D, cathepsin E, chymosin (or “rennin”), napsin, pepsin, plasmepsin, presenilin, renin, trypsin, chymotrypsin, elastase, and cysteine endoprotease (EP) B2 (also known as EPB2). In a preferred embodiment, the at least one additional protease is active at acidic pH, such as that found in the stomach (e.g., pH 1.5 to 3.5).

Combination with Nepenthesis Proteases

In some embodiments, the composition comprising a recombinant neprosin polypeptide includes an additional protease, which is a nepenthesis protease, such as one or more of nepenthesin I and nepenthesin II. Nepenthesin (EC 3.4.23.12) is a class of aspartic protease present in Nepenthes pitcher secretions, as well as a variety of other plant sources (see, e.g., Tokes et al., Digestive Enzymes Secreted by the Carnivorous Plant Nepenthes macferlanei L., Planta (Berl.), 1974, 119:39-46). Nepenthesin has two known isotypes: nepenthesin I (known to have two variants: nepenthesin Ia and nepenthesin Ib) and nepenthesin II. Nepenthesin I mRNA/cDNA sequences and corresponding amino acid sequences have been described from several Nepenthes species, including, among others, Nepenthes mirabilis (GenBank Accession No. JX494401 and AFV26024); Nepenthes gracilis (GenBank Accession No. AB114914 and BAD07474), and Nepenthes alata (GenBank Accession No. AB266803 and BAF98915). Nepenthesin II mRNA/cDNA sequences have been described from several Nepenthes species, including, among others, Nepenthes mirabilis (GenBank Accession No. JX494402 and AFV26025), and Nepenthes gracilis (GenBank Accession No. AB114915 and BAD07475.1). The sequences are also found in U.S. Patent Application Publication No. 20140186330, which is incorporated herein by reference in its entirety. Each of the sequences represented by the GenBank Accession Nos. provided herein are incorporated herein by reference in their entireties. Exemplary nepenthesin I and nepenthesin II proteases are presented as SEQ ID NO: 72, 73, 74, 75, 76, and 77. In some embodiments, biosynthesis of Nepenthes enzyme(s) can be achieved by transforming a cell with a vector comprising a cDNA that encodes a nepenthesin.

In some embodiments, the nepenthesin is a recombinant nepenthesin having at least about 85%, at least about 90%, at least about 95%, at least about 96%, at least about 97%, at least about 98%, or at least about 99% sequence identity to an amino acid sequence of nepenthesin I. In some embodiments, the nepenthesin comprises the amino acid sequence of nepenthesin I.

In some embodiments, the nepenthesin is a variant of nepenthesin having at least about 85%, at least about 90%, at least about 95%, at least about 96%, at least about 97%, at least about 98%, or at least about 99% sequence identity to an amino acid sequence of nepenthesin H. In some embodiments, the nepenthesin comprises the amino acid sequence of nepenthesin II.

In some embodiments, nepenthesin I, nepenthesin II or a variant thereof is synthesized by transfecting, infecting, or transforming a cell with one or more vectors comprising a cDNA sequence of each desired enzyme. In some embodiments, a single cell, cell line, or organism may be engineered so as to produce two or more enzymes. In some embodiments, the desired enzymes are synthesized by separate cells and combined in the pharmaceutical composition. In a preferred embodiment, the recombinant nepenthesin I, nepenthesin II, or a variant thereof is not glycosylated. In some embodiments, the recombinant nepenthesin I, nepenthesin II, or a variant thereof has a different glycosylation pattern than the natural enzyme (i.e., nepenthesin I or nepenthesin II, isolated from a Nepenthes plant). The synthetic (e.g., recombinant) Nepenthes enzyme(s) can be concentrated or purified according to known methods, such as those for isolating Nepenthes enzyme(s) from the plant pitcher liquid.

In some embodiments, the composition comprises neprosin and nepenthesin or variant thereof. In some embodiments, the composition comprises neprosin and nepenthesin I or variant thereof. In some embodiments, the composition comprises neprosin and nepenthesin II or variant thereof. In some embodiments, the composition comprises neprosin, nepenthesin I and nepenthesin II, or variant thereof. In some embodiments, the composition comprises a ratio of neprosin to nepenthesin (or variant of each thereof) of at least about 100:1. In some embodiments, the composition comprises a ratio of neprosin to nepenthesin of at least about 90:1. In some embodiments, the composition comprises a ratio of neprosin to nepenthesin of at least about 70:1. In some embodiments, the composition comprises a ratio of neprosin to nepenthesin of at least about 60:1. In some embodiments, the composition comprises a ratio of neprosin to nepenthesin of at least about 50:1. In some embodiments, the composition comprises a ratio of neprosin to nepenthesin of at least about 40:1. In some embodiments, the composition comprises a ratio of neprosin to nepenthesin of at least about 30:1. In some embodiments, the composition comprises a ratio of neprosin to nepenthesin of at least about 20:1. In some embodiments, the composition comprises a ratio of neprosin to nepenthesin of at least about 10:1. In some embodiments, the composition comprises a ratio of neprosin to nepenthesin of at least about 5:1. In some embodiments, the composition comprises a ratio of neprosin to nepenthesin of at least about 4:1. In some embodiments, the composition comprises a ratio of neprosin to nepenthesin of at least about 3:1. In some embodiments, the composition comprises a ratio of neprosin to nepenthesin of at least about 2:1. In some embodiments, the composition comprises a ratio of neprosin to nepenthesin of at least about 1:1. In some embodiments, the composition comprises a ratio of neprosin to nepenthesin of at least about 1:2. In some embodiments, the composition comprises a ratio of neprosin to nepenthesin of at least about 1:3. In some embodiments, the composition comprises a ratio of neprosin to nepenthesin of at least about 1:4. In some embodiments, the composition comprises a ratio of neprosin to nepenthesin of at least about 1:5. In some embodiments, the composition comprises a ratio of neprosin to nepenthesin of at least about 1:10. In some embodiments, the composition comprises a ratio of neprosin to nepenthesin of at least about 1:20. In some embodiments, the composition comprises a ratio of neprosin to nepenthesin of at least about 1:30. In some embodiments, the composition comprises a ratio of neprosin to nepenthesin of at least about 1:40. In some embodiments, the composition comprises a ratio of neprosin to nepenthesin of at least about 1:50. In some embodiments, the composition comprises a ratio of neprosin to nepenthesin of at least about 1:60. In some embodiments, the composition comprises a ratio of neprosin to nepenthesin of at least about 1:70. In some embodiments, the composition comprises a ratio of neprosin to nepenthesin of at least about 1:80. In some embodiments, the composition comprises a ratio of neprosin to nepenthesin of at least about 1:90. In some embodiments, the composition comprises a ratio of neprosin to nepenthesin of at least about 1:100.

In some embodiments, the composition comprises neprosin and substantially only recombinant nepenthesin I or variant thereof. In some embodiments, the composition comprises neprosin and substantially only nepenthesin II or variant thereof. In some embodiments, the composition comprises neprosin and nepenthesin I and nepenthesin II, or variant thereof. In some embodiments, the composition comprises neprosin and recombinant nepenthesin or variant thereof at a ratio of nepenthesin I to nepenthesin II (or variant of each thereof) of at least about 100:1. In some embodiments, the composition comprises neprosin and recombinant nepenthesin or variant thereof at a ratio of nepenthesin I to nepenthesin II of at least about 90:1. In some embodiments, the composition comprises neprosin and recombinant nepenthesin or variant thereof at a ratio of nepenthesin I to nepenthesin II of at least about 70:1. In some embodiments, the composition comprises neprosin and recombinant nepenthesin or variant thereof at a ratio of nepenthesin I to nepenthesin II of at least about 60:1. In some embodiments, the composition comprises neprosin and recombinant nepenthesin or variant thereof at a ratio of nepenthesin I to nepenthesin II of at least about 50:1. In some embodiments, the composition comprises neprosin and recombinant nepenthesin or variant thereof at a ratio of nepenthesin I to nepenthesin II of at least about 40:1. In some embodiments, the composition comprises neprosin and recombinant nepenthesin or variant thereof at a ratio of nepenthesin I to nepenthesin II of at least about 30:1. In some embodiments, the composition comprises neprosin and recombinant nepenthesin or variant thereof at a ratio of nepenthesin I to nepenthesin II of at least about 20:1. In some embodiments, the composition comprises neprosin and recombinant nepenthesin or variant thereof at a ratio of nepenthesin I to nepenthesin II of at least about 10:1. In some embodiments, the composition comprises neprosin and recombinant nepenthesin or variant thereof at a ratio of nepenthesin I to nepenthesin II of at least about 5:1. In some embodiments, the composition comprises neprosin and recombinant nepenthesin or variant thereof at a ratio of nepenthesin I to nepenthesin II of at least about 4:1. In some embodiments, the composition comprises neprosin and recombinant nepenthesin or variant thereof at a ratio of nepenthesin I to nepenthesin II of at least about 3:1. In some embodiments, the composition comprises neprosin and recombinant nepenthesin or variant thereof at a ratio of nepenthesin I to nepenthesin II of at least about 2:1. In some embodiments, the composition comprises neprosin and recombinant nepenthesin or variant thereof at a ratio of nepenthesin I to nepenthesin II of at least about 1:1. In some embodiments, the composition comprises neprosin and recombinant nepenthesin or variant thereof at a ratio of nepenthesin I to nepenthesin II of at least about 1:2. In some embodiments, the composition comprises neprosin and recombinant nepenthesin or variant thereof at a ratio of nepenthesin I to nepenthesin II of at least about 1:3. In some embodiments, the composition comprises neprosin and recombinant nepenthesin or variant thereof at a ratio of nepenthesin I to nepenthesin II of at least about 1:4. In some embodiments, the composition comprises neprosin and recombinant nepenthesin or variant thereof at a ratio of nepenthesin I to nepenthesin II of at least about 1:5. In some embodiments, the composition comprises neprosin and recombinant nepenthesin or variant thereof at a ratio of nepenthesin I to nepenthesin II of at least about 1:10. In some embodiments, the composition comprises neprosin and recombinant nepenthesin or variant thereof at a ratio of nepenthesin I to nepenthesin II of at least about 1:20. In some embodiments, the composition comprises neprosin and recombinant nepenthesin or variant thereof at a ratio of nepenthesin I to nepenthesin II of at least about 1:30. In some embodiments, the composition comprises neprosin and recombinant nepenthesin or variant thereof at a ratio of nepenthesin I to nepenthesin II of at least about 1:40. In some embodiments, the composition comprises neprosin and recombinant nepenthesin or variant thereof at a ratio of nepenthesin I to nepenthesin II of at least about 1:50. In some embodiments, the composition comprises neprosin and recombinant nepenthesin or variant thereof at a ratio of nepenthesin I to nepenthesin II of at least about 1:60. In some embodiments, the composition comprises neprosin and recombinant nepenthesin or variant thereof at a ratio of nepenthesin I to nepenthesin II of at least about 1:70. In some embodiments, the composition comprises neprosin and recombinant nepenthesin or variant thereof at a ratio of nepenthesin I to nepenthesin II of at least about 1:80. In some embodiments, the composition comprises neprosin and recombinant nepenthesin or variant thereof at a ratio of nepenthesin I to nepenthesin II of at least about 1:90. In some embodiments, the composition comprises neprosin and recombinant nepenthesin or variant thereof at a ratio of nepenthesin I to nepenthesin II of at least about 1:100.

In some embodiments, the ratio of neprosin to nepenthesin I and/or II in the composition is such that the peptidic food antigen is cleaved into sufficiently small and/or innocuous fragments so as to prevent, reduce, or attenuate gluten intolerance, celiac disease, wheat allergy, or dermatitis herpetiformis, inflammation, IEL proliferation or recruitment, intraepithelial lymphocytosis, and/or villous atrophy, or any symptom thereof, in an intestine of the subject. In some embodiments, the neprosin:nepenthesin ratio is between about 1:100 to about 100:1.

In some embodiments, the nepenthesin I or nepenthesin II in the composition is a full-length nepenthesin I or nepenthesin II. In some embodiments, the nepenthesin I or nepenthesin II in the composition is a mature form of nepenthesin I or nepenthesin II. In some embodiments, the nepenthesin I or nepenthesin II in the composition is a pro-enzyme of nepenthesin I or nepenthesin II.

With regards to pro-enzyme or pro-polypeptide of nepenthesin, it is known that nepenthesin I and/or nepenthesin II occur as pro-enzymes in Nepenthes and that the pro-polypeptide or pro-enzyme is cleaved in order to activate the enzyme in the pitcher fluid. In some embodiments, the composition comprises recombinant neprosin, in the forms described above, and a pro-enzyme/pro-peptide of nepenthesin I and/or nepenthesin II. In some embodiments, the pro-peptide is adjacent to the N terminus of the enzyme. In some embodiments, the pro-peptide is the naturally-occurring pro-peptide for the enzyme. In some embodiment, the pro-peptide is a heterologous pro-peptide (e.g., from a different protein or species, or synthetic). In some embodiment, the pro-peptide is cleaved by acidic conditions. In some embodiments, the pro-peptide is cleaved by an enzyme. In some embodiments, the presence of the pro-peptide results in delayed activity of the enzyme in the stomach (e.g., due to the time required to remove the pro-peptide and produce the mature enzyme). In some embodiments, the pro-peptide is engineered to be removed more slowly in order to delay activity of the enzyme in the stomach. In some embodiment, the pro-peptide is engineered to be removed more quickly in order to speed up activity of the enzyme in the stomach.

Uses and Methods

The recombinant neprosin polypeptide described herein, alone or as a composition with other proteases, can be used to degrade dietary proteins, particularly proline- and/or glutamine-rich proteins or other immunogenic proteins that are not effectively degraded by digestive tract enzymes. Degradation of such proteins would increase absorption of the proteins and/or decrease immunogenicity, which may have beneficial effects on the symptoms of intestinal diseases and disorders, e.g., celiac disease, gluten intolerance, irritable bowel syndrome, colitis, Crohn's disease, food allergies and the like. Accordingly, in a further aspect, the present disclosure provides methods for modulating a condition mediated by gluten intolerance in a subject, comprising administering to a subject in need thereof an effective amount of a recombinant neprosin polypeptide described herein. In some embodiments, the condition is celiac disease or a wheat allergy.

Without being bound by theory, it is believed that inflammatory response to gluten in the intestines of affected individuals is due to incomplete hydrolysis of gluten proteins, leading to the formation of toxic (immunotoxic) gluten peptides. The toxic properties of gluten proteins (e.g., gliadins and glutenins) are believed to be largely due to proline- and glutamine-rich peptides that are produced during incomplete degradation of the proteins by human digestive enzymes (including pepsin). Gastric and pancreatic endoproteases are unable to cleave these toxic or immunogenic peptide byproducts of incomplete degradation, at least in part because such enzymes lack specificity for proline and/or glutamine These toxic peptide byproducts include, but are not limited to, the “33-mer” and “p31-49” peptides. The toxic peptides are believed to cause numerous intestinal symptoms in sensitive individuals, including intraepithelial lymphocytosis, villous atrophy, and/or inflammation. Other proteins present in wheat also implicated in the autoimmune response, include serpins, purinins, alpha-amylase/protease inhibitors, globulins, and farinins.

Neprosin alone or in combination with other protease, such as nepenthesin I and/or nepenthesin II, are able to cleave toxic food peptides into smaller, non-toxic peptides, or at least peptides that have reduced immunogenicity. Because neprosin as well as the nepenthesin enzymes are active at abroad acidic pH range, digestion by the enzymes can initiate in the acidic environment of the stomach. Administration of neprosin and/or nepenthesin, in combination with a potentially antigenic food protein, results in a decrease in immune response to the antigenic food protein after ingestion, including a decrease in infiltration and/or production of intraepithelial lymphocytes in the intestine. Intraepithelial lymphocytes are T cells that are interspersed between epithelial cells of the large and small intestine. An increased T cell count is an early indicator of inflammation and is potentially associated with gluten intolerance, including celiac disease. Administration of recombinant neprosin or compositions thereof are capable of degrading food protein antigens to a level where the immune response in the intestine, as measured by IEL infiltration, is attenuated or eliminated when used in combination with food. IEL infiltration due to the presence of peptidic food antigen(s) is an early biological indicator of sensitivity to food antigen (e.g., gluten).

Accordingly, in one aspect, use of a recombinant neprosin polypeptide relates to a method for attenuating or preventing an immune response to food protein antigens in the intestine of a mammal, the method comprising administering to a subject in need thereof an effective amount of a recombinant neprosin polypeptide described herein. In some embodiments, the recombinant neprosin is administered alone or in combination with nepenthesin I and/or nepenthesin II. In some embodiments, the amount of the pharmaceutical composition is effective to attenuate or prevent IEL infiltration of the intestine due to the presence of the peptidic food antigen(s).

In some embodiments, use of a recombinant neprosin polypeptide relates to a method for attenuating or preventing intestinal inflammation due to the presence of peptidic food antigen(s) in the intestine of a subject, the method comprising administering to a subject in need thereof an effective amount of a recombinant neprosin polypeptide. As discussed above, intestinal inflammation is characterized by infiltration and/or proliferation of IELs in the intestine. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to attenuate or prevent intestinal inflammation due to the presence of the peptidic food antigen(s).

In some embodiments, use of a recombinant neprosin polypeptide relates to a method for attenuating or preventing intraepithelial lymphocytosis due to the presence of peptidic food antigen(s) in an intestine of a subject, the method comprising administering to a subject in need thereof an effective amount of a recombinant neprosin polypeptide. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to inhibit intraepithelial lymphocytosis in the intestine.

In some embodiments, use of a recombinant neprosin polypeptide relates to a method for attenuating or preventing production and/or recruitment of IELs in the intestine due to the presence of a peptidic food antigen in an intestine of a mammal, the method comprising administering to a subject in need thereof an effective amount of a recombinant neprosin polypeptide. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to degrade gluten protein so as to attenuate or prevent production and/or recruitment of IELs in the intestine.

In some embodiments, use of a recombinant neprosin polypeptide relates to a method for reducing T cell response to a peptidic food antigen, the method comprising contacting the peptidic food antigen with an effective amount of a recombinant neprosin polypeptide. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to so as to reduce T cell response to the antigen. In some embodiments, T cell response in an intestine of a mammal is reduced.

In some embodiments, use of a recombinant neprosin polypeptide relates to a method for attenuating or preventing villous atrophy due to the presence of a peptidic food antigen in an intestine of a subject, the method comprising administering to a subject in need thereof an effective amount of a recombinant neprosin polypeptide. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to degrade gluten protein so as to attenuate or prevent villous atrophy in the intestine. In some embodiments, the villous atrophy is a result of inflammation of the intestine.

In some embodiments, use of a recombinant neprosin polypeptide relates to a method of treating and/or ameliorating at least one symptom associated with an immune response to the presence of gluten or other antigenic protein in the intestine of a patient, the method comprising administering to a subject in need thereof an effective amount of a recombinant neprosin polypeptide to treat and/or ameliorate at least one symptom associated with an immune response to the presence of gluten or other antigenic protein. In some embodiments, such symptoms include, without limitation, “foggy mind”, depression, anxiety, ADHD-like behavior, abdominal pain, bloating, diarrhea, constipation, headaches, migraines, bone or joint pain, chronic fatigue, small intestine damage, development of tissue transglutaminase (tTG) antibodies, severe acne, vomiting, weight loss, irritability, iron-deficiency anemia, arthritis, tingling numbness in the extremities, infertility, and canker sores of the mouth. In some embodiments, the recombinant neprosin is administered alone or in combination with nepenthesin I and/or nepenthesin II.

In some embodiments, use of a recombinant neprosin polypeptide relates to a method for attenuating or preventing a manifestation of celiac disease arising from the presence of partially hydrolyzed gluten or wheat protein in an intestine of a patient having celiac disease, the method comprising administering to the patient an effective amount of a recombinant neprosin polypeptide described herein. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to so as to attenuate or prevent a manifestation of celiac disease.

In another aspect, provided are methods for treating gluten intolerance or an associated condition, such as celiac disease, wheat allergy, gluten sensitivity and dermatitis herpetiformis, in a patient in need thereof, comprising treating a food comprising gluten or suspected of comprising gluten with an effective amount of a recombinant neprosin polypeptide or composition thereof prior to consumption by the patient. In some embodiments, the food is combined with an effective amount of recombinant neprosin polypeptide or compositions thereof during its preparation. In some embodiments, the neprosin polypeptide is added after any heating steps in the food preparation. In some embodiments, the neprosin polypeptide is added before one or more heating steps in the food preparation. In some embodiments, the recombinant neprosin is administered alone or in combination with nepenthesin I and/or nepenthesin II.

In some embodiments, use of a recombinant neprosin polypeptide relates to a method for improving digestibility of a protein from a food in a subject with an intestinal disorder, the method comprising administering to the subject in need thereof an effective amount of a recombinant neprosin polypeptide. In some embodiments, the recombinant neprosin is administered alone or in combination with nepenthesin I and/or nepenthesin II. In some embodiments, the amount of neprosin, alone or in combination with nepenthesin I and/or nepenthesin II, is effective to degrade protein sufficiently to improve absorption of the protein in the intestine. In some embodiments, the intestinal disorder is Crohn's disease, irritable bowel syndrome, or colitis. In some embodiments, protein absorption from the food is increased.

In some embodiments, the recombinant neprosin or composition thereof is administered to the subject prior to ingestion of a potentially antigenic food or protein. In some embodiments, the recombinant neprosin or composition thereof is administered to the subject concurrently with ingestion of a potentially antigenic food or protein. In some embodiments, the recombinant neprosin or composition thereof is administered to the subject after ingestion of a potentially antigenic food or protein. In some embodiments, the recombinant neprosin or composition thereof is administered to the subject immediately after ingestion of a potentially antigenic food or protein. In some embodiments, the recombinant neprosin or composition thereof is administered to the subject irrespective of consumption of a potentially antigenic food or protein. In some embodiments, the potentially antigenic protein is gluten. In some embodiments, the potentially antigenic protein is one or more wheat proteins.

In some embodiments, the subject is a human patient. In some embodiments, the human patient selected for treatment suffers from gluten sensitivity or celiac disease. In some embodiments, the human patient suffers from celiac disease. In some embodiments, the human patient suffers from a disease selected from the group consisting of gluten intolerance, celiac disease, attention deficit hyperactivity disorder, autism, rheumatoid arthritis, fibromyalgia, and dermatitis herpetiformis. In some embodiments, the human patient suffers from a food allergy. In some embodiments, the intestinal antigen protein sensitivity correlates, directly or indirectly, with attention deficit hyperactivity disorder, autism, rheumatoid arthritis, fibromyalgia, and/or dermatitis herpetiformis. It is further contemplated that removing such antigenic intestinal proteins from the intestine using compositions of this invention will have a positive effect on attention deficit hyperactivity disorder, autism, rheumatoid arthritis, fibromyalgia, and/or dermatitis herpetiformis.

In some embodiments, the pharmaceutical composition is orally administered prior to, during, or immediately after consumption of a gluten-containing or other immunogenic food.

In some embodiments, the recombinant neprosin polypeptide or composition thereof is administered to the subject prior to ingestion by the subject of the food comprising gluten or suspected of comprising gluten. In some embodiments, the neprosin polypeptide or composition thereof is administered within a period that the enzyme is at least partially effective (for example, at least about 10%, 20%, 50%, 70%, 90% of original activity) in degrading gluten in the food that the subject will ingest. In some embodiments, the neprosin polypeptide or composition thereof is administered not more than about 4 hours, 3 hours, 2 hours, 1 hour, or 30 minutes prior to ingestion of the food by the subject.

In some embodiments, the neprosin polypeptide or composition thereof is administered to the subject concurrently with ingestion by the subject of the potentially immunogenic food. In some embodiments, the neprosin polypeptide or composition thereof is administered with the food. In some embodiments, the neprosin polypeptide or composition thereof is administered separately from the food.

In some embodiments, the neprosin polypeptide or composition thereof is administered to the subject shortly after ingestion by the subject of the potentially immunogenic food. In some embodiments, the neprosin polypeptide or composition thereof is administered within a period that at least part (for example, at least about 10%, 20%, 50%, 70%, 90%) of the antigen(s) in the food is still in the stomach of the subject. In some embodiments, the neprosin polypeptide or composition thereof is administered not more than 4 hours, 3 hours, 2 hours, 1 hour, or 30 minutes after ingestion of the food by the subject.

Generally, the neprosin polypeptide or composition thereof is administered in an amount that is safe and sufficient to produce the desired effect of detoxification of peptidic food antigen(s). The dosage of neprosin polypeptide or composition thereof can vary depending on factors such as the particular enzyme or combination administered, the subject's sensitivity to the food, the amount and types of antigen-containing food ingested, the pharmacodynamic properties of the enzyme, the mode of administration, the age, health and weight of the recipient, the nature and extent of the symptoms, the frequency of the treatment and the type of concurrent treatment, if any, and the clearance rate of the enzyme. One of skill in the art can determine the appropriate dosage based on the above factors. Neprosin polypeptide or composition thereof may be administered initially in a suitable dosage that may be adjusted as required, depending on the clinical response. In vitro assays may optionally be employed to help identify optimal dosage ranges. The precise dose to be employed in the formulation will also depend on the route of administration and/or the seriousness of the disease or disorder, and should be decided according to the judgment of the practitioner and each subject's circumstances.

In some embodiments, the dosage or dosing regimen of an adult subject may be proportionally adjusted for children and infants, and also adjusted for other administration or other formats, in proportion for example to molecular weight or immune response. Administration or treatments may be repeated at appropriate intervals, at the discretion of the physician.

In some embodiments, the effective amount of the recombinant neprosin polypeptide or composition thereof is between about 1 mg and about 1 g. In some embodiments, the effective amount is between about 1 mg and about 10 mg. In some embodiments, the effective amount of is less than about 5 mg per day. In some embodiments, the effective amount is between about 10 mg and about 1 g. In some embodiments, the effective amount is between about 20 mg and about 1 g. In one embodiment, the effective amount is between about 30 mg and about 1 g. In some embodiments, the effective amount is between about 40 mg and about 1 g. In some embodiments, the effective amount is between about 50 mg and about 1 g. In some embodiments, the effective amount is between about 60 mg and about 1 g. In some embodiments, the effective amount is between about 70 mg and about 1 g. In some embodiments, the effective amount is between about 80 mg and about 1 g. In some embodiments, the effective amount is between about 100 mg and about 1 g. In some embodiments, the effective amount is between about 500 mg and about 1 g. In some embodiments, the effective amount is between about 1 mg and about 500 mg. In some embodiments, the effective amount is between about 1 mg and about 250 mg. In some embodiments, the effective amount is between about 1 mg and about 200 mg. In some embodiments, the effective amount is between about 1 mg and about 100 mg. In some embodiments, the effective amount is between about 1 mg and about 90 mg. In some embodiments, the effective amount is between about 1 mg and about 80 mg. In some embodiments, the effective amount is between about 1 mg and about 70 mg. In some embodiments, the effective amount is between about 1 mg and about 60 mg. In some embodiments, the effective amount is between about 1 mg and about 50 mg. In some embodiments, the effective amount is between about 1 mg and about 40 mg. In some embodiments, the effective amount is between about 1 mg and about 30 mg. In some embodiments, the effective amount is between about 1 mg and about 20 mg. In some embodiments, the effective amount is between about 1 mg and about 5 mg. In some embodiments, the effective amount is between about 1 mg and about 4 mg. In some embodiments, the effective amount is between about 1 mg and about 3 mg. This includes any values within any of these ranges (including endpoints), and subranges between any two of these values.

In some embodiments, the recombinant neprosin polypeptide is administered in dosages of about 0.001 mg to about 1000 mg of enzyme per kg body weight per day, or about 1 mg to about 100 g per dose for an average person. In some embodiments, recombinant neprosin polypeptide can be administered at 0.001, 0.01, 0.1, 1, 5, 10, 50, 100, 500, or 1000 mg/kg body weight per day, and ranges between any two of these values (including endpoints). In some embodiments, recombinant neprosin polypeptide can be administered at 1 mg, 10 mg, 100 mg, 200 mg, 500 mg, 700 mg, 1 g, 10 g, 20 g, 50 g, 70 g, 100 g per dose, and ranges between any two of these values (including endpoints). In some embodiments, recombinant neprosin polypeptide may be administered once, twice, three times, etc. a day, depending on the number of times the subject ingests a food comprising an antigenic protein and/or how much of such food is consumed. The amount of recombinant neprosin polypeptide recited herein may relate to total enzyme or each enzyme in the composition,

In some embodiments, the amount of recombinant neprosin polypeptide administered is based on the amount (or approximate amount) of substrate (e.g., gluten and/or other protein or potentially antigenic protein) consumed/to be consumed. In some embodiments, about 1 mg to about 1 g of enzyme is administered per 1 g of substrate. In some embodiments, about 5 mg to about 1 g of enzyme is administered per 1 g of substrate. In some embodiments, about 10 mg to about 1 g of enzyme is administered per I g of substrate. In some embodiments, about 100 mg to about 1 g of enzyme is administered per 1 g of substrate. In some embodiments, about 1 mg to about 500 mg of enzyme is administered per 1 g of substrate. In some embodiments, about 1 mg to about 250 mg of enzyme is administered per 1 g of substrate. In some embodiments, about 1 mg to about 100 mg of enzyme is administered per 1 g of substrate. In some embodiments, about 1 mg to about 10 mg of enzyme is administered per 1 g of substrate. This includes any values with these ranges (including endpoints), and subranges between any two of these values,

In a preferred embodiment, the ratio of substrate to enzyme is between about 10:1 and about 1000:1. In some embodiments, the ratio of substrate to enzyme is between about 10:1 and about 100:1.

In some embodiments, the ratio of substrate (total protein) to enzyme (neprosin or combination of Nepenthes enzymes) administered is between about 1:1 and about 15000:1. In some embodiments, the ratio of substrate to enzyme is between about 10:1 and about 15000:1. In some embodiments, the ratio of substrate to enzyme is between about 100:1 and about 15000:1. In some embodiments, the ratio of substrate to enzyme is between about 500:1 and about 15000:1. In some embodiments, the ratio of substrate to enzyme is between about 1000:1 and about 15000:1. In some embodiments, the ratio of substrate to enzyme is between about 5000:1 and about 15000:1. In some embodiments, the ratio of substrate to enzyme is between about 10000:1 and about 15000:1. In some embodiments, the ratio of substrate to enzyme is between about 1:1 and about 10000:1. In some embodiments, the ratio of substrate to enzyme is between about 10:1 and about 10000:1. In some embodiments, the ratio of substrate to enzyme is between about 100:1 and about 10000:1. In some embodiments, the ratio of substrate to enzyme is between about 500:1 and about 10000:1. In some embodiments, the ratio of substrate to enzyme is between about 1000:1 and about 10000:1. In some embodiments, the ratio of substrate to enzyme is between about 5000:1 and about 10000:1. In some embodiments, the ratio of substrate to enzyme is between about 1:1 and about 5000:1. In some embodiments, the ratio of substrate to enzyme is between about 10:1 and about 5000:1. In some embodiments, the ratio of substrate to enzyme is between about 100:1 and about 5000:1. In some embodiments, the ratio of substrate to enzyme is between about 500:1 and about 5000:1. In some embodiments, the ratio of substrate to enzyme is between about 1000:1 and about 5000:1. In some embodiments, the ratio of substrate to enzyme is between about 1:1 and about 1000:1. In some embodiments, the ratio of substrate to enzyme is between about 10:1 and about 1000:1. In some embodiments, the ratio of substrate to enzyme is between about 100:1 and about 1000:1. In some embodiments, the ratio of substrate to enzyme is between about 1:1 and about 500:1. In some embodiments, the ratio of substrate to enzyme is between about 10:1 and about 500:1. In some embodiments, the ratio of substrate to enzyme is between about 100:1 and about 500:1. In some embodiments, the ratio of substrate to enzyme is between about 1:1 and about 100:1. In some embodiments, the ratio of substrate to enzyme is between about 10:1 and about 100:1. This includes any values within any of these ranges (including endpoints), and subranges between any two of these values.

In some embodiments, the total protein may be, for example, the total protein consumed at a given meal, or the total protein consumed in a specific period of time (e.g., in an hour, 2 hours, or 3-24 hours). In some embodiments, the total protein is the total of all protein consumed by the subject in a 1-hour period. In some embodiments, the total protein is the total of all protein consumed by the subject in a 2-hour period. In some embodiments, the total protein is the total of all protein consumed by the subject in a 3-hour period. In some embodiments, the total protein is the total of all protein consumed by the subject in a 4-hour period. In some embodiments, the total protein is the total of all protein consumed by the subject in a 5-hour period. In some embodiments, the total protein is the total of all protein consumed by the subject in a 10-hour period. In some embodiments, the total protein is the total of all protein consumed by the subject in a 12-hour period. In some embodiments, the total protein is the total of all protein consumed by the subject in a 15-hour period. In some embodiments, the total protein is the total of all protein consumed by the subject in a 20-hour period. In some embodiments, the total protein is the total of all protein consumed by the subject in a 24-hour period.

In some embodiments, the pharmaceutical composition is administered irrespective of whether the patient has ingested (e.g., knowingly ingested) a food containing a potentially immunogenic protein. In some embodiments, the pharmaceutical composition is administered on an as-needed basis, e.g., before, during, and/or after a meal that might be contaminated by a potentially immunogenic protein, or in which the potentially immunogenic protein content is unknown. In some embodiments, the pharmaceutical composition is administered on a regular basis. In some embodiments, the pharmaceutical composition is administered at least one time per day. In some embodiments, the pharmaceutical composition is administered two, three, four, or more times per day. In some embodiments, the pharmaceutical composition is administered in conjunction with (e.g., before, during, or after) each meal and/or snack. In some embodiments, the pharmaceutical composition is included as part of a sustained release formulation where there is a continuous release of enzyme(s) to allow for intermittent snacking, etc. without regard to the antigenic protein content of the food.

In some embodiments and as discussed herein, the recombinant neprosin polypeptide can be administered as the sole active agent or it can be administered in combination with other agents (simultaneously, sequentially or separately, or through co-formulation), including other compounds that demonstrate the same or a similar therapeutic activity and that are determined to safe and efficacious for such combined administration. In some embodiments, as described above, the pharmaceutical. composition is administered with an additional enzyme, such as a gastric protease, an aspartic protease (such as pepsin, pepsinogen or those described by Chen et al., Gene, 2009, 442:108-118), and enzymes such as another prolyl endopeptidase (PEP), dipeptidyl peptidase IV (DPP IV), and dipeptidyl carboxypeptidase (DCP) or cysteine proteinase B (see, e.g., U.S. Pat. No. 7,910,541), as described herein. In some embodiments, the additional enzyme is administered in the form of bacteria that produce and/or secrete the additional enzyme. In some embodiments, the bacteria are engineered to produce and/or secrete neprosin, alone or with nepenthesin I and/or nepenthesin II.

In some embodiments, the pharmaceutical composition is administered to the subject with another therapeutic agent which is not a protease. Non-limiting examples of agents that can be administered with the pharmaceutical composition include inhibitors of tissue transglutaminase, anti-inflammatory agents such as amylases, glucoamylases, endopeptidases, HMG-CoA reductase inhibitors (e.g., compactin, lovastatin, simvastatin, pravastatin and atorvastatin), leukotriene receptor antagonists (e.g., montelukast and zafirlukast), COX-2 inhibitors (e.g., celecoxib and rofecoxib), p38 MAP kinase inhibitors (e.g., BIRB-796); mast cell-stabilizing agents such as sodium chromoglycate (chromolyn), pemirolast, proxicromil, repirinast, doxantrazole, amlexanox nedocromil and probicromil, anti-ulcer agents, anti-allergy agents such as anti-histamine agents (e.g., acrivastine, cetirizine, desloratadine, ebastine, fexofenadine, levocetirizine, loratadine and mizolastine), inhibitors of transglutaminase 2 (TG2), anti-TNFα agents, and antibiotics. In some embodiments, the additional agent is aprobiotic. Probiotics include, without limitation, lactobacillus, yeast, bacillus, or bifidobacterium species and strains. In some embodiments, the other agent is elafin. In some embodiments, the other agent is administered in the form of bacteria that produce and/or secrete the additional agent.

### EXAMPLES

The following Examples, including experiments and results achieved, are provided for illustrative purposes only and are not to be construed as limiting the present invention.

### Example 1

### Expression of Neprosin in Mammalian Cells

On the day of transfection, viable cell density and percent viability were determined for the prepared cells. Prewarmed Expi293™ Expression Medium was used to dilute the cells to a final viable cell density of 3×106 viable cells/mL, and percent viability should be >95%. The culture flasks were swirled gently to mix the cells to prepare for transfection. Transient transfection was performed using the recommended protocol from the ExpiFectamine™ 293 Transfection Kit. Viable cell density and percent viability was examined every two days. When the viable cell density was less than half of the peak maximum, or when the viable cell percent viability is under 70%, the culture was harvested. Neprosin I (Npr1) is secreted into the media and harvesting of the supernatant was performed on the 5th day post transfection. The entire culture was centrifuged with a benchtop refrigerated centrifuge at 4,000 RCF for 30 minutes in 50 mL falcon tubes and the filter supernatant was filtered using a 0.22-μm filters. Select samples were subjected to purification.

| | Estimated Yield | | |
|---|---|---|---|
| | on 50 mL, Post | Host | SEQ |
| | purification | Expression | ID NO |
| Construct | (mg) | Cell Line | (nt/aa) |
| mSP-His-GST-Npr1 | 1.53 | Expi293 | 35/36 |
| mSP-Npr1 | ND | Expi293 | 37/38 |
| mSP-His-Npr1 | 6.59 | Expi293 | 39/40 |
| mSP-His-noAP_Npr1 | 0 | Expi293 | 41/42 |
| mSP-His-Npr1-N68A, | 2.47 | Expi293 | 43/44 |
| N253A, N152A | | | |
| mSP--His-Npr1-N68A | 5.43 | Expi293 | 45/46 |
| mSP-His-Npr1-N152A | 5.25 | Expi293 | 47/48 |
| mSP--His-Npr1-N253A | 4.73 | Expi293 | 49/50 |
| mSP--His-Npr1-N68A, | 3.42 | Expi293 | 51/52 |
| N253A | | | |

### Example 2

### Activation of Recombinant Neprosin and Activity on Gliadin Substrate

Formation of Active Neprosin Protease Polypeptide

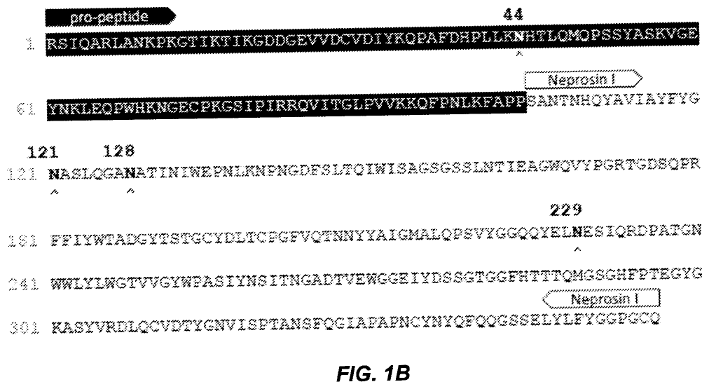

An 80 μL aliquot of ˜40 μM Expi293 or ExpiCHO purified Npr1 (prepared from cells expressing mSP-His-Npr1; SEQ ID NO: 40) were added to a 3.5K MWCO Slide-a-lyzer MINI Dialysis Device (Thermo Scientific, USA) containing 14 mL of 100 mM glycine, pH 2.5. The dialysis device was incubated at 37° C., 250 rpm. At the incubated time points, 4 μL of the enzyme were removed and diluted 1:5 into 1× Mcllvaine buffer, pH 7.5. Immediately after the dilution, the sample was mixed 1:1 with 2×SDS-PAGE sample buffer. Samples in SDS-PAGE buffer were heated for 10 min at 95° C., and 10 μL was loaded onto a 12 well NuPAGE gel (4-12% Bis-Tris). SDS-PAGE was run at 200V for 35 min and gels stained with Coomassie Blue. Results are shown in FIG. 2.

pH Activation Profile of Neprosin Protease Polypeptide

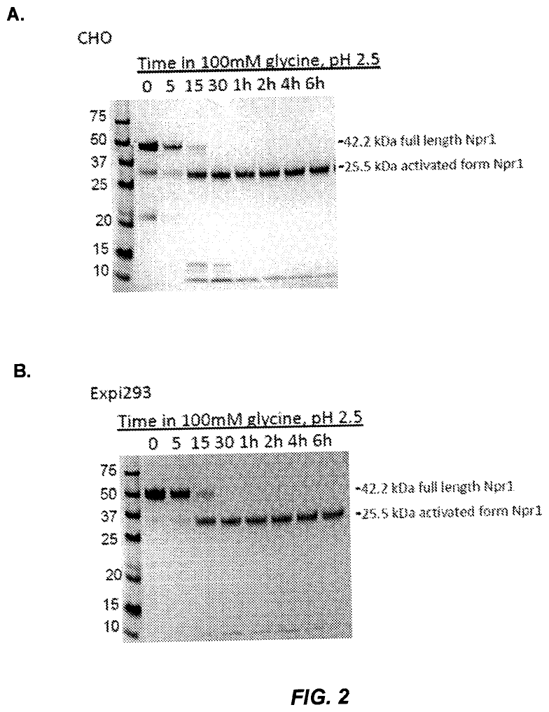

A 50 μL aliquot of ˜8 μM purified Npr1 (mSP-His-Npr1; SEQ ID NO: 40) were added to a 3.5K MWCO Slide-a-lyzer MINI Dialysis Device containing 14 mL of 1× Macllvaine buffer at specified pHs (i.e., pH 2.5, 3, 3.5, 4, 4.5, or 5) or 1× Simulated Gastric Fluid (SGF; 7 mM KCl, 1 mM KH₂PO₄, 47 mM NaCl, 0.1 mM MgCl₂(H₂O)₆, 0.075 mM CaCl₂(H₂O)₂)±0.8 g/L pepsin. The dialysis device was incubated at 37° C., 250 rpm. At 60 min, the 50 jut enzyme sample was recovered and placed into a PCR tube. Ten jut of these samples were immediately mixed 1:1 with 2×SDS-PAGE sample buffer. The remaining sample was left at RT for 5 hrs, without shaking, and then mixed with sample buffer as above. Samples in SDS-PAGE buffer were heated for 10 min at 95° C., and 10 jut was loaded onto a 12 well NuPAGE gel (4-12% Bis-Tris). SDS-PAGE was run at 200V for 35 min and gels stained with Coomassie Blue. Results are shown in FIG. 3.

Activity of Activated Neprosin Protease on Gliadin Substrate

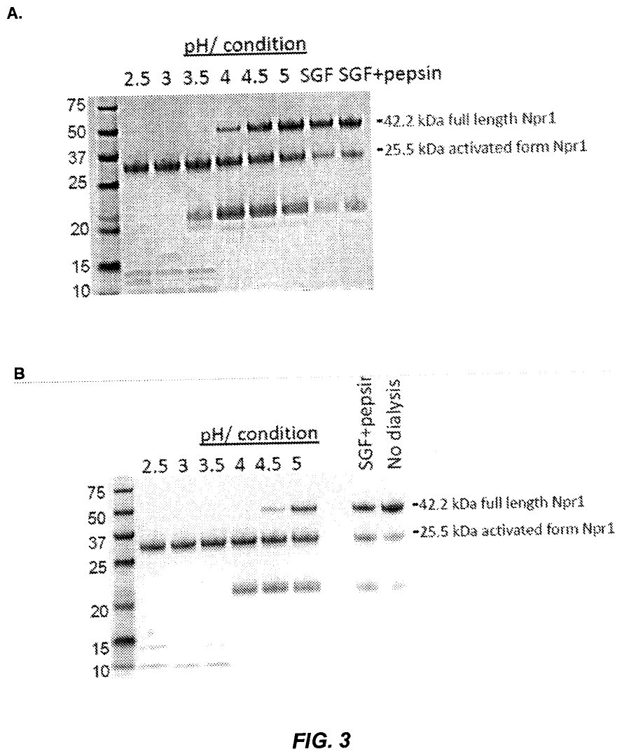

A ˜1.2 mg/mL solution of solubilized gliadin stock (previously prepared and frozen) was centrifuged at 15,000 rpm and the soluble fraction transferred to a new tube. The soluble fraction was diluted 1:10 in ddH₂O to yield a ˜0.12 mg/mL working stock. A 50 jut aliquot of ˜0.12 mg/mL gliadin were added to 40 μL of 100 mM sodium acetate buffer, pH 4 in a PCR tube. An aliquot of Neprosin enzyme, 10 μL of 0.06 mg/mL or 0.006 mg/mL enzyme preparation, were added to start the reaction and incubated at 37° C., 900 rpm for 5 min or 30 min. The reaction was quenched by placing reaction tubes in a pre-heated (95° C.) thermocycler and heating at 95° C. for 10 min. Samples were diluted 15,000× before being analyzed using a RIDASCREEN® Gliadin competitive ELISA (Samples containing no enzyme were used to calculate the percent of gliadin remaining Results are shown in FIG. 4. Neprosin preparation activated by treatment at acidic pH displays significantly greater proteolytic activity compared to unactivated neprosin polypeptide prepared from cells expressing mSP-His-Npr1.

### Example 3

### Transfection with and Expression of Neprosin in Pichia Cells

Transfection of Pichia cells with the Neprosin gene

200 ng of Pme1-linearized DNA were added to 30 μL of Pichia BG10 electrocompetent cells and mixed gently. Cell and DNA mix was transferred to a chilled 1 mm electroporation cuvette and electroporated using 25 μF, 200 SI, and 1150 V (EXM 630, BTX). 1 mL of Pichia Electroporation Recovery Solution (YPD: 1M sorbitol, 1:1 (v/v)) was added to the cuvette, mixed gently with cell solution, and transferred to a different tube. Electroporated cells were incubated at 20° C. for 3 h, 100 rpm. 100 jut of incubated cells were then spread onto YPD+800 μg/mL G418 agar plates at multiple dilutions to obtain single colonies, and incubated at 30° C. for 48 h. To generate glycerol stocks, single colonies were picked and used to inoculate 2 mL BMY media containing 1.5% glycerol (BMGY) and grown at 30° C. for 48 h, 250 rpm. Cells were stored in 25% glycerol at −80° C.

Expression of Neprosin in Pichia

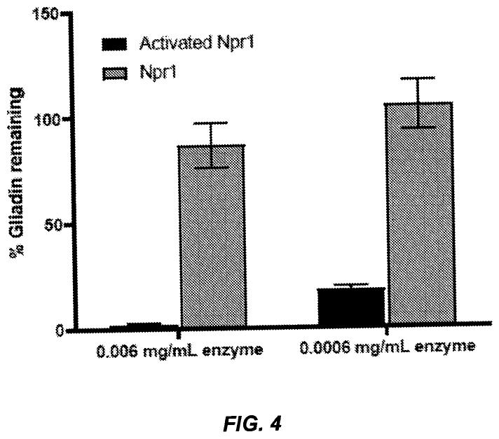

A glycerol stock of Pichia cells containing the Neprosin (Npr1) gene was used to streak a YPD+800 μg/mL G418 agar plate. The plate was incubated at 30° C. for 48 h. A single colony from the agar plate was used to inoculate 50 mL BMGY. This starter culture was incubated at 30° C. for 24 h, 200 rpm and subsequently used to inoculate (1:50) BMGY-containing shake flasks. Cultures were allowed to grow at 30° C. for 48 h, 250 rpm. Cells were collected by centrifugation and then resuspended in BMY media containing 2% methanol (BMMY) to induce protein expression. Secreted Npr1 was obtained by harvesting the supernatant 24 h later (4,000 rpm, 20 min). The supernatant was filtered using 0.45-μm filters and subjected to purification. SDS-PAGE analysis of WT, N216A, and N216A, N324A Npr1 (SEQ ID: 56, 60, and 66, respectively) is shown in FIG. 5.

| | Estimated Yield | | |
|---|---|---|---|
| | on 2 L, Post | Host | SEQ |
| | purification | Expression | ID NO |
| Construct | (mg) | Cell Line | (nt/aa) |
| pJAG-s1-Npr1 | ND | Pichia BG10 | 53/54 |
| pJAG-s1-His-Npr1 | 80 | Pichia BG10 | 55/56 |
| pJAG-s1-His-N139A Npr1 | ND | Pichia BG10 | 57/58 |
| pJAG-s1-His-N216A Npr1 | 54 | Pichia BG10 | 59/60 |
| pJAG-s1-His-N223A Npr1 | ND | Pichia BG10 | 61/62 |
| pJAG-s1-His-N324A Npr1 | ND | Pichia BG10 | 63/64 |
| pJAG-s1-His-N216A, N324A | 78 | Pichia BG10 | 65/66 |
| Npr1 | | | |
| pJAG-s1-His-N139A, N324A, | ND | Pichia BG10 | 67/68 |
| N223A, Npr1 | | | |
| pJAG-s1-His-N139A, N216A, | ND | Pichia BG10 | 69/70 |
| N223A, N324A Npr1 | | | |

### Example 4

### Activation of Recombinant Neprosin from Pichia and Activity on Gliadin Substrate

Formation of Active Neprosin Protease Polypeptide

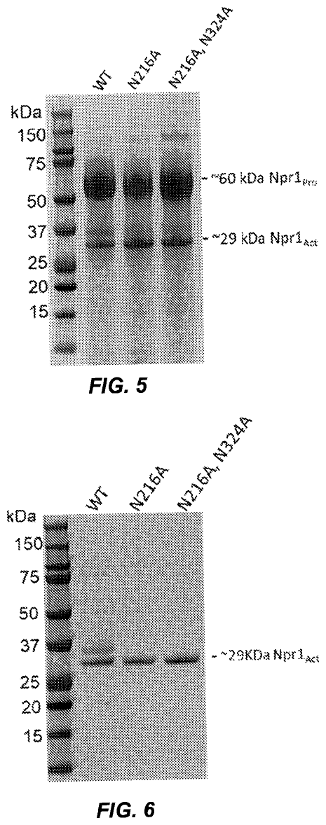

WT Npr1, N216A Npr1, or N216A/N324A Npr1 prepared from Pichia cells (SEQ ID:54, 60, and 66, respectively) was added to a 10K MWCO Slide-A-Lyzer dialysis cassette (Thermo Scientific, USA) and dialyzed against 100 mM glycine, pH 2.5 for 4 h, 37° C., light stirring. Samples of the dialyzed proteins were mixed 1:1 with 2×SDS-PAGE sample buffer and heated at 95° C. for 10 min. 10 μL of samples were loaded onto a 10 well NuPAGE gel (4-12% Bis-Tris). SDS-PAGE was run at 200V for 35 min and gels stained with Coomassie Blue. Results are shown in FIG. 6. The activated WT neprosin appears as two glycosylated polypeptides around 29 and 35 kDa. Activated N216A neprosin and N216A/N324A neprosin appear as single glycosylated species around 29 kDa.

N-terminal sequencing on the activated WT neprosin bands using in-gel digestion LC-MS/MS analysis (Thermo Orbitrap Exploris 240) identified P199-S200 (SEQ ID 54) as the cleavage site between pro-peptide and activated Npr1. This analysis also confirmed the identity of both protein bands as activated Npr1, their difference in molecular weight being due to mannose-chain length glycosylation differences.

Activity of Activated Pichia Neprosin on Gliadin Substrate

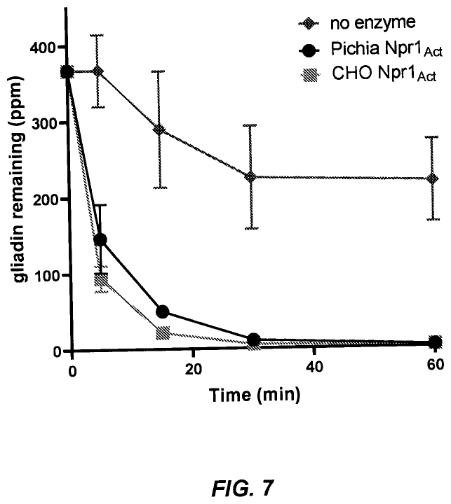

A ˜32 mg/mL solution of solubilized gliadin stock (previously prepared and frozen) was centrifuged at 15,000 rpm and the soluble fraction transferred to a new tube. The soluble fraction was diluted 1:1 in ddH2O to yield a ˜16 mg/mL working stock. 10 jut of ˜16 mg/mL gliadin were added to 80 jut of 10× Simulated Gastric Fluids solution (SGF: Mcllvaine buffer, pH 3, 8 mg/mL pepsin) in a PCR tube. 10 μL of 0.016 mg/mL activated Pichia WT Npr1 enzyme preparation, were added to start the reaction and incubated at 37° C., 900 rpm for 60 min, with samples collected at 0, 5, 15, 30, and 60 min. The reaction was quenched by placing reaction tubes in a pre-heated (95° C.) thermocycler and heating at 95° C. for 10 min. Samples were diluted 1,000× before being analyzed using a RIDASCREEN® Gliadin competitive ELISA (R-Biopharm AG, Germany) Samples containing no neprosin enzyme or activated neprosin protein prepared in CHO cells were run in parallel for comparison. Results are shown in FIG. 7. The activated neprosin protease from Pichia is proteolytically active on gliadin substrate in SGF and shows equivalent gliadin digestion activity when compared to the enzyme prepared from mammalian CHO cells.

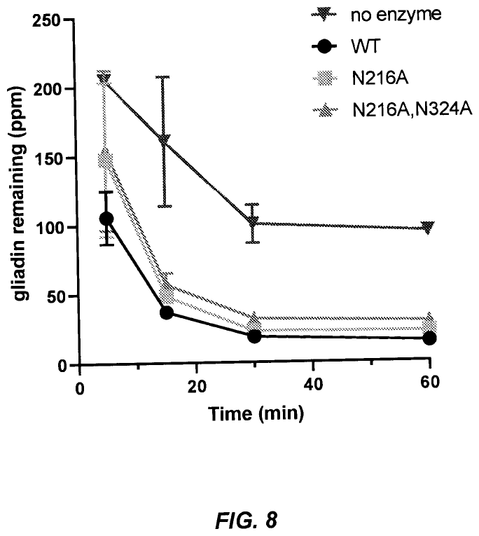

The activity of N216A Npr1 and N216A/N324A Npr1 prepared from Pichia on gliadin was tested in the same manner as just described. Samples containing no neprosin enzyme or WT neprosin protein prepared in Pichia cells were run in parallel for comparison. Results are shown in FIG. 8. Both N216A Npr1 and N216A/N324A Npr1 digested gliadin in a similar manner as WT Npr1 produced in Pichia.

### Example 5

### Activity on Gliadin Substrate in a Mouse Stomach

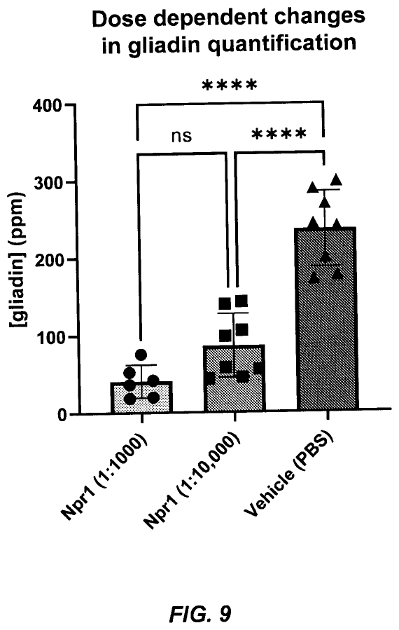

Gliadin substrate (Sigma) at 60 mg/mL was prepared by solubilizing in 25 mM glycine HCl, pH 2.5 (stirring, 55° C., 3 h), and ensured a homogenous solution by bath sonication for three times for 3 min. A gliadin substrate dosing solution of 40 mg/mL gliadin in 2% methylcellulose (400 cP) was then prepared by end-over-end mixing, 18 h, 25° C. Eight-week-old, female, C57Bl/6 mice maintained on a gluten-free diet for at least 5 days were fasted for 4 h prior to dosing Animals were orally dosed with 5 mg of the gliadin dosing solution followed by CHO WT Npr1 (SEQ ID: 40) at a 1:1000 or 1:10,000 enzyme-to-substrate ratio 2 min post-substrate dose. Fifteen mins post enzyme dose, mice were euthanized (CO2 chamber). Stomachs contents were collected by flushing with 1 mL of 100 mm HEPES, pH 7. Luminal contents were then heated at 95° C. for 10 min. Samples were diluted 1,000× before being analyzed using a RIDASCREEN® Gliadin competitive ELISA (R-Biopharm AG, Germany) Results are shown in FIG. 9.

### Example 6

### Prevention of Celiac Disease Patient T-Cell Activation

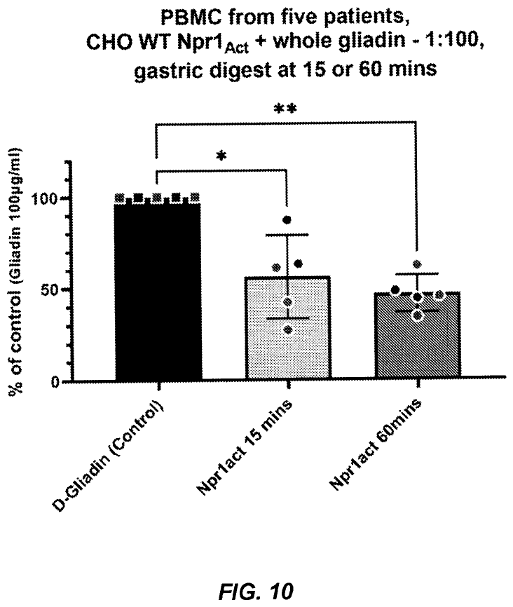

Gliadin substrate (Sigma) at 60 mg/mL was prepared by solubilizing in 25 mM glycine HCl, pH 2.5 (stirring, 55° C., 3 h), and ensured a homogenous solution by bath sonication for three times for 3 min. The substrate was submitted to a 15 min simulated gastric digest (pH 4, 0.8 mg/mL pepsin, 37° C.) with and without CHO WT Npr1 (SEQ ID: 40) at a 1:100 enzyme-to-substrate ratio, followed by a 30 min simulated intestinal digest (pH 7, 0.15 mg/mL trypsin, 0.15 mg/mL chymotrypsin, 37° C.) and then heat inactivated at 95° C. for 10 min. The resultant mixture was deamidated using transglutaminase (tTG) (Sigma) (pH 7, 2 h, 37° C.) and heat inactivated again at 95° C. for 10 min. Digested substrate was diluted to 100 μg/mL in sterile PBS and combined with thawed PBMCs (peripheral blood mononuclear cells) isolated from celiac disease participants on a strict gluten-free diet with HLA-DQ2.5+ restriction who consent to and have undergone 3-day oral gluten challenge. Antigen and PBMC (2.5-5×10⁵ cells/well) were added to the wells of ELISpot plates coated with IFN-gamma antibody and incubated overnight (16-20 h). Secreted IFN-gamma is detected via a biotinylated antibody-streptavidin complex after development with BCIP substrate. ELISpot plates were read using an automated spot reader. Results in the FIG. 10 are expressed as a percentage of the positive control (buffer plus gliadin digested with natural enzymes) normalized for each patient.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. A recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to the pre-pro-polypeptide, pro-polypeptide, mature polypeptide, or polypeptide of SEQ ID NO: 1, wherein the amino acid sequence comprises at least at amino acid position 68, 145, 152, or 253, or combinations thereof, and equivalent positions thereof, a substitution with an amino acid other than asparagine (N), wherein the amino acid positions are relative to the reference sequence corresponding to SEQ ID NO: 1.

2. The recombinant neprosin polypeptide of claim 1, wherein the amino acid sequence of the recombinant neprosin polypeptide comprises at least a substitution at least at amino acid position 68 with an amino acid other than asparagine (N).

3. The recombinant neprosin polypeptide of claim 1 wherein the amino acid sequence of the recombinant neprosin polypeptide comprises at least a substitution at least at amino acid position 152 with an amino acid other than asparagine (N).

4. The recombinant neprosin polypeptide of claim 1, wherein the amino acid sequence of the recombinant neprosin polypeptide comprises at least a substitution at least at amino acid position 253 with an amino acid other than asparagine (N).

5. The recombinant neprosin polypeptide of claim 1, wherein the amino acid sequence of the recombinant neprosin polypeptide comprises at least a substitution at least at amino acid position 145 with an amino acid other than asparagine (N).

6. The recombinant neprosin polypeptide of claim 1, wherein the signal sequence of the pre-pro-polypeptide is replaced with a signal sequence functional in a mammalian cell, insect cell, or fungal cell, or wherein the pro-polypeptide further comprises a signal sequence functional in a mammalian cell, insect cell, or fungal cell.

7. A recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 105 to 356 of SEQ ID NO: 8, or to a reference sequence corresponding to SEQ ID NO: 8, wherein the amino acid sequence comprises at least at amino acid position 44, 121, 128, or 229, or combinations thereof, a substitution with an amino acid other than asparagine (N), wherein the amino acid positions are relative to SEQ ID NO: 8.

8. (canceled)

9. The recombinant neprosin polypeptide of claim 7, wherein the amino acid sequence of the recombinant neprosin polypeptide comprises SEQ ID NO: 10.

10. (canceled)

11. The recombinant neprosin polypeptide of claim 7, wherein the amino acid sequence of the recombinant neprosin polypeptide comprises residues 105 to 356 of SEQ ID NO: 12, or comprises SEQ ID NO: 12.

12. (canceled)

13. The recombinant neprosin polypeptide of claim 7, wherein the amino acid sequence of the recombinant neprosin polypeptide comprises residues 105 to 356 of SEQ ID NO: 14, or comprises SEQ ID NO: 14.

14. (canceled)

15. The recombinant neprosin polypeptide of claim 7, wherein the amino acid sequence of the recombinant neprosin polypeptide comprises residues 105 to 356 of SEQ ID NO: 16, or comprises SEQ ID NO: 16.

16. (canceled)

17. The recombinant neprosin polypeptide of claim 7, wherein the amino acid sequence of the recombinant neprosin polypeptide comprises residues 105 to 356 of SEQ ID NO: 18, or comprises SEQ ID NO: 18.

18. (canceled)

19. The recombinant neprosin polypeptide of claim 7, wherein the amino acid sequence of the recombinant neprosin polypeptide comprises residues 105 to 356 of SEQ ID NO: 20, or comprises SEQ ID NO: 20.

20. (canceled)

21. The recombinant neprosin polypeptide of claim 7, wherein the amino acid sequence of the recombinant neprosin polypeptide comprises residues 105 to 356 of SEQ ID NO: 22, or comprises SEQ ID NO: 22.

22. (canceled)

23. The recombinant neprosin polypeptide of claim 7, wherein the amino acid sequence of the recombinant neprosin polypeptide comprises residues 105 to 356 of SEQ ID NO: 32, or comprises SEQ ID NO: 32.

24-27. (canceled)

28. A recombinant neprosin polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 105 to 356 of SEQ ID NO: 8, or to a reference sequence corresponding to SEQ ID NO: 8, wherein the amino acid sequence further comprises a signal sequence functional in a mammalian cell, insect cell, or fungal cell.

29. The recombinant polynucleotide of claim 28, wherein the amino acid sequence of the recombinant neprosin polypeptide comprises residues 105-356 of SEQ ID NO: 8, or comprises SEQ ID NO: 8.

30. A recombinant polynucleotide comprising a polynucleotide sequence encoding a recombinant neprosin polypeptide of claim 1.

31-33. (canceled)

34. An expression vector comprising a recombinant polynucleotide of claim 30.

35. (canceled)

36. (canceled)

37. A host cell comprising an expression vector of claim 34, wherein the host cell is a eukaryotic cell.

38-42. (canceled)

43. A method of expressing recombinant neprosin, comprising culturing a host cell of claim 37 under suitable culture conditions such that the recombinant neprosin polypeptide is expressed.

44-66. (canceled)

67. A method of preparing mature neprosin polypeptide, comprising treating in vitro a neprosin pre-pro-polypeptide or pro-polypeptide of a recombinant neprosin polypeptide of claim 1, to a pH of about 2 to about 6 under suitable conditions for formation of the mature neprosin polypeptide.

68-70. (canceled)

71. A pharmaceutical composition comprising a pro-polypeptide or mature neprosin polypeptide of a recombinant neprosin polypeptide of claim 1.

72. (canceled)

73. A method of treating a disease or condition associated with gluten intolerance, comprising administering to a subject in need thereof an effective amount of a pro-polypeptide or proteolytically active mature neprosin polypeptide of a recombinant neprosin polypeptide of claim 1.

74-78. (canceled)


SEQUENCE LISTING (109 sequences)

SEQ ID NO: 1 - protein, 380 aa, Nepenthes x ventrata
MQAKFFTFVILSSVFYFNYPLAEARSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 2 - protein, 380 aa, Nepenthes alata
MQVKLFTFVILSSIFYFNYHLAKARSIQARLANKPKGTIKTIKGDDGEMVDCVDVYKQPAFDHPLLKNPSLQMQPNLYTSQFDEYNKLKQSWHKSGQCPKCSIPIRRQVISGVPVVRKQFPNLKFAPPSANTNHQYVVIEYNYDSATLQGANATINIWDPTLKNPNKDFSLTQIWISAGSGSNLNTIEAGWQVYPRRTGDSQPRFFIYWTADGYSSTGCYDLTCPGFVQTNNYYAIGMALQPSVYGGQQFELNESIQRDPATGNWWLYLWGTVVGYWPASIYNSITNGADTVEWGGEIYDSSGTGGFHTTTQMGSGHFPTEGYGKASYVRDLQCVDTYGNVISPPTNSFQEIAPAPNCYNYQFQQGSSELYLFYGGPGCQ

SEQ ID NO: 3 - protein, 419 aa, Nepenthes ampullaria
MAVPFSKYLVTAAFIVVLFLWVSISLTSAARSTAASREKIEVHKHLKRLNKPAVHSVQSPDGDVIDCVHISHQPAFDHPFLRNVTVQHRPSYHPEGLFDENNKIVTTPKERTNPITLWHLNGRCPEGTVPIRRTKKDDILRASSVKRFGRKPHRSIPEPRSADPDLVNESGQHHAIAYVEGDKYYGAKATINVWEPQIQQSNEFSLSQIWVLGGSFGEDLNSIEAGWQVSPDLYGDNNTRLFTYWTSDAYQATGCYNLLCSGFIQINNEIAMGASISPVSAYRNSQYDISILVWKDPKEGNWWMQFGNNYVLGYWPAFLFSYLADSASMIEWGGEVVNSEPDGRHTSTMGSGHFPEEGFGRSSYFRNIQVVDASNNLKAPEDIGTFTEQSNCYDVQTGNNGDWGHYFYYGGPGRNPNCP

SEQ ID NO: 4 - protein, 420 aa, N. ampullaria
MTSQFSGDLMRRAAFGVLFFSWVSVSLTCAAGFPAASRQKLDVAKHLKRLNKPALHTIQSPDGDIIDCVHISRQPAFDHPFLKNHTIQIRPSYHPEGLFDENKIATEQKERINPITQLWHLNGKCPEGTIPIRRTKKNDILRASSVKRYGRKKHRSVPQPKSADPDIVNESGHQHAIAYVEGDKYYGAKATINVWEPQIQQPNEFSLSQIWVLGGSFGEDLNSIEAGWQVSPDLYGDNNTRLFTYWTSDAYATGCYNLLCSGFIQINNEIAMGASISPVSAYRNSQYDISILVWKDPKEGNWWMQFGKDYILGYWPSLLFSYLADGASMIEWGGEVVNSEPGGDHTSTQMGSGHFPEEGFGRASYFRNIQVVDGSNILKAPRDVGTFTEQSNCYAVQTGNNGDWGFHFYYGGPGRNPNCP

SEQ ID NO: 5 - protein, 359 aa, Nepenthes rafflesiana
MGAACRMLLLLVAAFLLNSSFIHGRDIIARKLAKPPKATIKTIRWDNGDIFDCVDIYKQPAFEHPKLKNHKLQMEPSSYPFGVRVKNVSEDKLGPWHKSGKCPEGSIPIRRQVITGPIIRKNTTFGSPASYNNGHQYAVIHYFTQSQIIQGAQATIDIWDPSLANPQGDFSLSQIWYIAGSGSNLNTAEAGWQVYPARTGDSQPRLFVYWTNQYTPGMALTPSTYNGQQNELDLVINRDTTGNFLYVEGTAIGYWPNSIYNILQAGSDRVDWGGEILDSSGSDGFHTLTQMGSGYFPSAGYGLSSYIRDLFYTEDTQNWVAVVANALQGTAPAPYCYNYALENGQNGALWLFYGGPGCK

SEQ ID NO: 6 - protein, 388 aa, Nepenthes rafflesiana
MFKKASTMQAKFFTFVILSSVFYFNYPLAEARSIQARLANKPKGTIKTIKGDDGEVVDCVDIYKQPAFDHPLLKNHTLQMQPSSYASKVGEYNKLEQPWHKGNECPKGSIPIRRQVITGLPVVKKQFPNLKFAPPSANTNHQYAVIAYYYGSASLQGANATINIWDPTLENPNGDFSLTQIIWIVAGSGSSLNTIEAGWQVYPGRTGDSQPRFFIYWTADGYTSTGCYDLTCPGFVQTNNYYAIGMALQPSVYGGQQFELNESIQRDRTTGNWWLYLWGTVVGYWPASIYNSITNGADTVEWGGEIYDSSGTGGFNITTQMGSGHFPSEGYGKASYVRDLQCVDTYGNVISPSANSFQGIAPAPNCYKYQFQQGSSELFLFYGGPGCH

SEQ ID NO: 7 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 8 - protein, 356 aa, synthetic construct
  REGION 1..356: note = Synthetic
RSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 9 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaaggcccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 10 - protein, 356 aa, synthetic construct
  REGION 1..356: note = Synthetic
RSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 11 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 12 - protein, 356 aa, synthetic construct
  REGION 1..356: note = Synthetic
RSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGAASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 13 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 14 - protein, 356 aa, synthetic construct
  REGION 1..356: note = Synthetic
RSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 15 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggccgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 16 - protein, 356 aa, synthetic construct
  REGION 1..356: note = Synthetic
RSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 17 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggccgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 18 - protein, 356 aa, synthetic construct
  REGION 1..356: note = Synthetic
RSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGAASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 19 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaaggctcacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggctgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 20 - protein, 356 aa, synthetic construct
  REGION 1..356: note = Synthetic
RSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 21 - DNA, 1068 bp, synthetic construct
  misc_feature 1..1068: note = Synthetic
  source 1..1068: mol_type = other DNA
cgctccattcaagctcgtttggccaacaaaccaaagggtaccatcaagactatcaagggtgatgatggtgaggttgtggactgtgttgatatttacaagcaaccagcttttgaccatccattgttgaaggctcacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggctgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 22 - protein, 356 aa, synthetic construct
  REGION 1..356: note = Synthetic
RSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGAASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 23 - DNA, 1074 bp, synthetic construct
  misc_feature 1..1074: note = Synthetic
  source 1..1074: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaatga

SEQ ID NO: 24 - protein, 356 aa, synthetic construct; identical to SEQ ID NO: 8
  REGION 1..356: note = Synthetic

SEQ ID NO: 25 - DNA, 1071 bp, synthetic construct
  misc_feature 1..1071: note = Synthetic
  source 1..1071: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 26 - protein, 356 aa, synthetic construct; identical to SEQ ID NO: 10
  REGION 1..356: note = Synthetic

SEQ ID NO: 27 - DNA, 1071 bp, synthetic construct
  misc_feature 1..1071: note = Synthetic
  source 1..1071: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcagcagccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 28 - protein, 356 aa, synthetic construct; identical to SEQ ID NO: 14
  REGION 1..356: note = Synthetic

SEQ ID NO: 29 - DNA, 1071 bp, synthetic construct
  misc_feature 1..1071: note = Synthetic
  source 1..1071: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 30 - protein, 356 aa, synthetic construct; identical to SEQ ID NO: 16
  REGION 1..356: note = Synthetic

SEQ ID NO: 31 - DNA, 1071 bp, synthetic construct
  misc_feature 1..1071: note = Synthetic
  source 1..1071: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 32 - protein, 356 aa, synthetic construct
  REGION 1..356: note = Synthetic
RSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 33 - DNA, 1071 bp, synthetic construct
  misc_feature 1..1071: note = Synthetic
  source 1..1071: mol_type = other DNA
agatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcagcagccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 34 - protein, 356 aa, synthetic construct; identical to SEQ ID NO: 20
  REGION 1..356: note = Synthetic

SEQ ID NO: 35 - DNA, 1833 bp, synthetic construct
  misc_feature 1..1833: note = Synthetic
  source 1..1833: mol_type = other DNA
atggaatggagctgggtctttctcttcttcctgtcagtaacgactggtgtccactcccaccaccatcatcatcatggaggatctcccatcctgggctactggaagattaaaggtcttgtgcagccaaccagattgctcctggaatatctggaagaaaagtacgaggagcacctgtatgaaagggacgaaggcgataaatggcggaacaagaagttcgagctggggttggagtttcctaatctgccgtactacatagacggcgatgtcaagctcacacaaagcatggccatcatccgctacattgcagacaaacacaacatgttgggagggtgccccaaagagcgagctgagatctctatgcttgaaggtgccgttctcgatattaggtatggagtgtcacgcatagcgtatagtaaggacttcgagactctgaaggtggattttctgagcaaactgcctgaaatgctgaagatgttcgaggaccggctgtgtcacaagacgtaccttaatggcgaccatgtaacccacccagattttatgctctatgatgctttggatgtcgtcctgtacatggaccctatgtgccttgacgcattccccaaactcgtgtgttttaagaagagaatcgaagccattccacagatcgacaaatatctcaaatcctccaagtacattgcttggccacttcagggttggcaggccacattcggcggcggcgatcaccctcccaaatctgacgagaacctgtactttcaaggtagatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaatga

SEQ ID NO: 36 - protein, 609 aa, synthetic construct
  REGION 1..609: note = Synthetic
MEWSWVFLFFLSVTTGVHSHHHHHHGGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 37 - DNA, 1128 bp, synthetic construct
  misc_feature 1..1128: note = Synthetic
  source 1..1128: mol_type = other DNA
atggaatggagctgggtctttctcttcttcctgtcagtaacgactggtgtccactccagatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 38 - protein, 375 aa, synthetic construct
  REGION 1..375: note = Synthetic
MEWSWVFLFFLSVTTGVHSRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 39 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 40 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 41 - DNA, 816 bp, synthetic construct
  misc_feature 1..816: note = Synthetic
  source 1..816: mol_type = other DNA
atggaatggagctgggtctttctcttcttcctgtcagtaacgactggtgtccactcccaccaccatcatcatcatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 42 - protein, 271 aa, synthetic construct
  REGION 1..271: note = Synthetic
MEWSWVFLFFLSVTTGVHSHHHHHHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 43 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcagcagccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 44 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 45 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 46 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 47 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcagcagccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctgaatgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 48 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 49 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaagaaccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 50 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 51 - DNA, 1146 bp, synthetic construct
  misc_feature 1..1146: note = Synthetic
  source 1..1146: mol_type = other DNA
atggaatggagctgggtctttctcttcttcctgtcagtaacgactggtgtccactcccaccaccatcatcatcatagatctattcaggctaggctggcaaataagcctaaaggcaccatcaagacaatcaaaggagacgacggtgaggtggtggattgcgtcgacatatacaagcagccagccttcgatcacccgctcctcaaggcccatacgctgcaaatgcagcctagcagctatgcctccaaagttggcgaatacaacaaactggagcagccatggcacaagaatggagaatgtcccaaagggtccattcccatacgccgccaggtgatcactggcttgcccgtagtaaagaagcagtttcctaacctgaaattcgctcctccaagtgcgaataccaaccatcaatatgcagtcatcgcctacttctatgggaatgcttcactgcagggtgcaaacgccacaatcaacatttgggagcccaatctgaagaatccgaacggcgatttttctctcacccagatttggatctcagccggaagcgggtcctccctgaacactattgaagctggctggcaggtgtacccaggtcgaacaggagacagtcaacctcggttctttatctattggaccgccgatgggtacacttctacaggctgctacgaccttacctgtcccgggtttgtgcagacgaataactattatgccataggaatggcattgcaaccaagcgtttacggtggccagcagtacgagctggctgagtcaattcaaagagatcccgctactggaaactggtggctctatttgtgggggacagtggtcggctactggcctgcgagtatctataatagcattaccaacggtgccgacaccgttgaatggggaggcgagatctacgattcttctggaactggtggattccacacaacaacccagatgggctccggccactttccaacggaaggctacggaaaagcaagctatgtgagggaccttcaatgcgtcgatacttacggcaatgtgatttcacccaccgccaacagtttccagggtatagctcctgcgccgaactgttacaattatcagtttcagcagggaagttccgagctgtacctgttctatggcggcccaggttgccagtaa

SEQ ID NO: 52 - protein, 381 aa, synthetic construct
  REGION 1..381: note = Synthetic
MEWSWVFLFFLSVTTGVHSHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 53 - DNA, 1338 bp, synthetic construct
  misc_feature 1..1338: note = Synthetic
  source 1..1338: mol_type = other DNA
atgagattcccaagcatttttactgccgttttatttgctgcttcttctgctttggccgctcctgtcaataccactactgaggacgaaactgctcaaattccagctgaggctgtcattggttacttggatttggaaggagacttcgatgttgccgttttaccattctccaactctaccaacaacggtttgctctttatcaacaccactatcgcttccattgctgccaaggaggaaggtgtttctttggacaagagagaagctgaagctcgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccagtga

SEQ ID NO: 54 - protein, 445 aa, synthetic construct
  REGION 1..445: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEARSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 55 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 56 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 57 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaaggcccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 58 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 59 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 60 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGAASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 61 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattgaacgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 62 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELNESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 63 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggccgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 64 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGNASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 65 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaagaaccacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccaatgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggccgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 66 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKNHTLQMQPSSYASKVGEYNKLEQPWHKNGECPKGSIPIRRQVITGLPVVKKQFPNLKFAPPSANTNHQYAVIAYFYGAASLQGANATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 67 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaaggctcacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtaatgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggctgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 68 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGNASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 69 - DNA, 1353 bp, synthetic construct
  misc_feature 1..1353: note = Synthetic
  source 1..1353: mol_type = other DNA
atgagattcccatctattttcactgctgttttgttcgctgcttcttctgctttggctgctccagttaacactactactgaggacgagactgctcaaattccagctgaggctgttattggttacttggacttggagggtgacttcgacgttgctgttttgccattctctaactctactaacaacggtttgttgttcattaacactactattgcttctattgctgctaaggaggagggtgtttctttggacaagagagaggctgaggctcaccatcatcaccatcaccgctccattcaagctcgtttggccaacaaaccaaagggtaccatcaagactatcaagggtgatgatggtgaggttgtggactgtgttgatatttacaagcaaccagcttttgaccatccattgttgaaggctcacactttgcaaatgcagccatcttcttacgcctccaaagttggcgaatacaacaagttggagcaaccatggcacaagaacggtgaatgcccaaaaggttccattcctattagaagacaagtcattactggtcttcctgttgtcaagaagcaattccccaatttgaagtttgctcctccatctgccaataccaatcaccaatacgctgttattgcttacttttacggtgctgcctctttgcaaggtgccgctgccactatcaacatttgggaaccgaatttgaagaacccaaacggtgatttctctttgactcaaatctggatttctgctggttccggttcttctttaaacaccattgaagctggttggcaagtttaccctggtagaactggtgattctcagccaagattcttcatctactggaccgctgatggttacacctccactggttgttacgatctgacttgccctggtttcgttcaaaccaacaattactacgccattggtatggcccttcaaccatctgtttacggtggtcagcaatatgaattggctgaaagtattcaaagagatcctgctactggaaactggtggttgtacttgtggggtaccgttgtcggttactggcccgcttccatctacaactcaatcaccaatggtgctgacactgttgaatggggtggtgaaatttacgactcttccggaacgggtggtttccacaccactactcaaatgggttctggtcacttcccaaccgaaggttacggtaaggcttcttacgtcagagatttgcaatgtgttgatacctatggtaatgtcatttctccaactgccaactcattccaaggtatcgccccagctccaaattgttacaactatcagtttcaacaaggttcctccgagttatacctcttttacggtgggccaggatgccag

SEQ ID NO: 70 - protein, 451 aa, synthetic construct
  REGION 1..451: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEAHHHHHHRSIQARLANKPKGTIKTIKGDDGEVVDCVDIYKQPAFDHPLLKAHTLQMQPSSYASKVGEYNKLEQPWHKNGECPKGSIPIRRQVITGLPVVKKQFPNLKFAPPSANTNHQYAVIAYFYGAASLQGAAATINIWEPNLKNPNGDFSLTQIWISAGSGSSLNTIEAGWQVYPGRTGDSQPRFFIYWTADGYTSTGCYDLTCPGFVQTNNYYAIGMALQPSVYGGQQYELAESIQRDPATGNWWLYLWGTVVGYWPASIYNSITNGADTVEWGGEIYDSSGTGGFHTTTQMGSGHFPTEGYGKASYVRDLQCVDTYGNVISPTANSFQGIAPAPNCYNYQFQQGSSELYLFYGGPGCQ

SEQ ID NO: 71 - DNA, 1234 bp, Nepenthes gracilis
  source 1..1234: mol_type = other DNA
acgtcaagaacagctctcaatcaccgtcacgaagccaaagtaacgggctttcagataatgcttgaacatgttgattcgggcaaaaacttaaccaaattccagctcttagaacgtgctatcgaaaggggtagtcgtagattgcagaggctcgaagccatgttaaatggcccctccggtgtggaaacttccgtctacgccggagatggcgaatatctgatgaacttatcgattggaactccggcacaacctttctccgcaatcatggataccggtagcgatcttatctggacgcagtgccagccttgcactcagtgttttaatcaatcaacgcccatatttaatcctcaaggatcatcctccttctccaccctcccttgctcaagccaactctgtcaagccctttcaagcccgacatgctctaataatttctgccaatacacctacgggtatggggacgggtccgaaacccaaggatccatgggcactgagactctcactttcgggtcggtttccatccctaatatcacattcggctgcggggaaaacaaccaagggtttgggcaaggaaacggggcaggcttggttgggatgggtcggggccctctgtcgcttccttctcaactcgtcgtgaccaaattctcttactgcatgacccccattggtagctcaacccctagcactcttctattgggatcactggctaattctgtcaccgccggtagtcctaatacaaccctaatccaaagctctcaaataccaactttctattatattactctcaacgggttgagtgttggttcaactcgcttgcccattgatccgagtgcttttgcacttaatagcaataatggaacaggagggataataatagactctggaacgacacttacttacttcgttaacgcttatcaatctgtaaggcaagagttcatctcccagattaatctacccgtcgtaaatggttcctcctccggctttgatctgtgcttccagacgccttctgatccgtcaaacctgcagatacccacctttgtgatgcattttgacggtggagatttggagttgcccagtgagaattatttcatctccccaagcaacgggctgatttgcttggcgatggggagttcgtcgcaggggatgtccatttttgggaatattcagcagcaaaacatgctagtcgtttacgacaccggaaattcggtggtttcattcgcttctgctcaatgtggtgcgt

SEQ ID NO: 72 - protein, 341 aa, Nepenthes gracilis
GPLGSMERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTIAENQAKNEHLQKENERLLRDWNDVQGRFEKCVSAKEALETDLYKRFILVLNEKKTKIRSLHNKLLNAAQEREKDIKQEGETAICSEMTADRDPVYDESTDEESENQTDLSGLASAAVSKDDSIISSLDVTDIAPSRKRRQRMQRNLGTEPKMAPQENQLQEKENSRPDSSLPETSKKEHISAENMSLETLRNSSPEDLFDEI

SEQ ID NO: 73 - protein, 437 aa, Nepenthes mirabilis
MASSLYSFLLALSIVYIFVAPTHSTSRTALNHHHEPKVAGFQIMLEHVDSGKNLTKFELLERAVERGSRRLQRLEAMLNGPSGVETPVYAGDGEYLMNLSIGTPAQPFSAIMDTGSDLIWTQCQPCTQCFNQSTPIFNPQGSSSFSTLPCSSQLCQALQSPTCSNNSCQYTYGYGDGSETQGSMGTETLTFGSVSIPNITFGCGENNQGFGQGNGAGLVGMGRGPLSLPSQLDVTKFSYCMTPIGSSTSSTLLLGSLANSVTAGSPNTTLIESSQIPTFYYITLNGLSVGSTPLPIDPSVFKLNSNNGTGGIIIDSGTTLTYFADNAYQAVRQAFISQMNLSVVNGSSSGFDLCFQMPSDQSNLQIPTFVMHFDGGDLVLPSENYFISPSNGLICLAMGSSSQGMSIFGNIQQQNLLVVYDTGNSVVSFLFAQCGAS

SEQ ID NO: 74 - protein, 437 aa, Nepenthes alata
MASSLYSFLLALSIVYIFVAPTHSTSRTALNHHHEPKVAGFQIMLEHVDSGKNLTKFELLERAVERGSRRLQRLEAMLNGPSGVETPVYAGDGEYLMNLSIGTPAQPFSAIMDTGSDLIWTQCQPCTQCFNQSTPIFNPQGSSSFSTLPCSSQLCQALQSPTCSNNSCQYTYGYGDGSETQGSMGTETLTFGSVSIPNITFGCGENNQGFGQGNGAGLVGMGRGPLSLPSQLDVTKFSYCMTPIGSSNSSTLLLGSLANSVTAGSPNTTLIQSSQIPTFYYITLNGLSVGSTPLPIDPSVFKLNSNNGTGGIIIDSGTTLTYFVDNAYQAVRQAFISQMNLSVVNGSSSGFDLCFQMPSDQSNLQIPTFVMHFDGGDLVLPSENYFISPSNGLICLAMGSSSQGMSIFGNIQQQNLLVVYDTGNSVVSFLSAQCGAS

SEQ ID NO: 75 - protein, 437 aa, Nepenthes gracilis
MASSLYSFLLALSIVYIFVAPTHSTSRTALNHRHEAKVTGFQIMLEHVDSGKNLTKFQLLERAIERGSRRLQRLEAMLNGPSGVETSVYAGDGEYLMNLSIGTPAQPFSAIMDTGSDLIWTQCQPCTQCFNQSTPIFNPQGSSSFSTLPCSSQLCQALSSPTCSNNFCQYTYGYGDGSETQGSMGTETLTFGSVSIPNITFGCGENNQGFGQGNGAGLVGMGRGPLSLPSQLDVTKFSYCMTPIGSSTPSNLLLGSLANSVTAGSPNTTLIQSSQIPTFYYITLNGLSVGSTRLPIDPSAFALNSNNGTGGIIIDSGTTLTYFVNNAYQSVRQEFISQINLPVVNGSSSGFDLCFQTPSDPSNLQIPTFVMHFDGGDLELPSENYFISPSNGLICLAMGSSSQGMSIFGNIQQQNMLVVYDTGNSVVSFASAQCGAS

SEQ ID NO: 76 - protein, 437 aa, Nepenthes mirabilis
MASPLHSVVLGLAIVSAIVAPTSSTSRGTLLHHGQKRPQPGLRVVLEQVDSGMNLTKYELIKRAIKRGERRMRSINAMLQSSSGIETPVYAGSGEYLMNVAIGTPASSLSAIMDTGSDLIWTQCEPCTQCFSQPTPIFNPQDSSSFSTLPCESQYCQDLPSESCYNDCQYTYGYGDGSSTQGYMATETFTFETSSVPNIAFGCGEDNQGFGQGNGAGLIGMGWGPLSLPSQLGVGQFSYCMTSSGSSSPSTLALGSAASGVPEGSPSTTLIHSSLNPTYYYITLQGITVGGDNLGIPSSTFQLQDDGTGGMIIDSGTTLTYLPQDAYNAVAQAFTDQINLSPVDESSSGLSTCFQLPSDGSTVQVPEISMQFDGGVLNLGEENVLISPAEGVICLAMGSSSQQGISIFGNIQQQETQVLYDLQNLAVSFVPTQCGAS

SEQ ID NO: 77 - protein, 438 aa, Nepenthes gracilis
MASPLYSVVLGLAIVSAIVAPTSSTSRGTLLHHGQKRPQPGLRVDLEQVDSGKNLTKYELIKRAIKRGERRMRSINAMLQSSSGIETPVYAGDGEYLMNVAIGTPDSSFSAIMDTGSDLIWTQCEPCTQCFSQPTPIFNPQDSSSFSTLPCESQYCQDLPSETCNNNECQYTYGYGDGSTTQGYMATETFTFETSSVPNIAFGCGEDNQGFGQGNGAGLIGMGWGPLSLPSQLGVGQFSYCMTSYGSSSPSTLALGSAASGVPEGSPSTTLIHSSLNPTYYYITLQGITVGGDNLGIPSSTFQLQDDGTGGMIIDSGTTLTYLPQDAYNAVAQAFTDQINLPTVDESSSGLSTCFQQPSDGSTVQVPEISMQFDGGVLNLGEQNILISPAEGVICLAMGSSSQLGISIFGNIQQQETQVLYDLQNLAVSFVPTQCGAS

SEQ ID NO: 78 - protein, 25 aa, synthetic construct
  REGION 1..25: note = Synthetic
MGVLLTQRTLLSLVLALLFPSMASM

SEQ ID NO: 79 - protein, 16 aa, synthetic construct
  REGION 1..16: note = Synthetic
MKCLLYLAFLFIGVNC

SEQ ID NO: 80 - protein, 21 aa, synthetic construct
  REGION 1..21: note = Synthetic
METDTLLLWVLLLWVPGSTGD

SEQ ID NO: 81 - protein, 19 aa, synthetic construct
  REGION 1..19: note = Synthetic
MGWSCIILFLVATATGVHS

SEQ ID NO: 82 - protein, 17 aa, synthetic construct
  REGION 1..17: note = Synthetic
MRAWIFFLLCLAGRALA

SEQ ID NO: 83 - protein, 21 aa, synthetic construct
  REGION 1..21: note = Synthetic
MWWRLWWLLLLLLLLWPMVWA

SEQ ID NO: 84 - protein, 22 aa, synthetic construct
  REGION 1..22: note = Synthetic
MDMRVPAQLLGLLLLWLRGARC

SEQ ID NO: 85 - protein, 16 aa, synthetic construct
  REGION 1..16: note = Synthetic
MPLLLLLPLLWAGALA

SEQ ID NO: 86 - protein, 23 aa, synthetic construct
  REGION 1..23: note = Synthetic
MDAMKRGLCCVLLLCGAVFVSPS

SEQ ID NO: 87 - protein, 18 aa, synthetic construct
  REGION 1..18: note = Synthetic
MAFLWLLSCWALLGTTFG

SEQ ID NO: 88 - protein, 15 aa, synthetic construct
  REGION 1..15: note = Synthetic
MNLLLILTFVAAAVA

SEQ ID NO: 89 - protein, 20 aa, synthetic construct
  REGION 1..20: note = Synthetic
MYRMQLLSCIALSLALVTNS

SEQ ID NO: 90 - protein, 17 aa, synthetic construct
  REGION 1..17: note = Synthetic
MGVKVLFALICIAVAEA

SEQ ID NO: 91 - protein, 16 aa, synthetic construct
  REGION 1..16: note = Synthetic
MKWVTFISLLFSSAYS

SEQ ID NO: 92 - protein, 16 aa, synthetic construct
  REGION 1..16: note = Synthetic
MKTIIALSYIFCLVLG

SEQ ID NO: 93 - protein, 24 aa, synthetic construct
  REGION 1..24: note = Synthetic
MALWMRLLPLLALLALWGPDPAAA

SEQ ID NO: 94 - protein, 16 aa, synthetic construct
  REGION 1..16: note = Synthetic
MKPIFLVLLVVTSAYA

SEQ ID NO: 95 - protein, 38 aa, synthetic construct
  REGION 1..38: note = Synthetic
MLLVNQSHQGFNKEHTSKMVSAIVLYVLLAAAAHSAFA

SEQ ID NO: 96 - protein, 19 aa, synthetic construct
  REGION 1..19: note = Synthetic
MEWSWVFLFFLSVTTGVHS

SEQ ID NO: 97 - protein, 89 aa, synthetic construct
  REGION 1..89: note = Synthetic
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 98 - protein, 88 aa, synthetic construct
  REGION 1..88: note = Synthetic
MRFPSIFTAVLFAASSALAAPANTTTFDETAQIPAFAVIDYSDLFGDFDAAALPLSNSTNNGISSTNTTIASIAAKEFGVLEKRFAFA

SEQ ID NO: 99 - protein, 19 aa, synthetic construct
  REGION 1..19: note = Synthetic
MRFPSIFTAVLFAASSALA

SEQ ID NO: 100 - protein, 13 aa, synthetic construct
  REGION 1..13: note = Synthetic
MLLQAFLFLLAGF

SEQ ID NO: 101 - protein, 130 aa, synthetic construct
  REGION 1..130: note = Synthetic
MKPLILSAILITLCITSIAVSAPVEGIFADLHDSSHPYITDDVGVRMSAIKEENPDRHFVGDEIPDEAVIQSFKSKRREILFLLDDIGSAVEFIGSNLAQIEANHLSERVQFSWTHIRKNQPFGKREAEA

SEQ ID NO: 102 - protein, 20 aa, synthetic construct
  REGION 1..20: note = Synthetic
MKAFTSLLCGLGLSTTLAKA

SEQ ID NO: 103 - protein, 46 aa, synthetic construct
  REGION 1..46: note = Synthetic
MDSEPLLPNPNDSRKPANWRRIIKYISLTLAWIGIFSYVYIYHGTA

SEQ ID NO: 104 - protein, 22 aa, synthetic construct
  REGION 1..22: note = Synthetic
MFSLKALLPLALLLVSANQVAA

SEQ ID NO: 105 - protein, 19 aa, synthetic construct
  REGION 1..19: note = Synthetic
MLLQAFLFLLAGFAAKISA

SEQ ID NO: 106 - protein, 31 aa, synthetic construct
  REGION 1..31: note = Synthetic
MLSLKPSWLTLAALMYAMLLVVVPFAKPVRA

SEQ ID NO: 107 - protein, 23 aa, synthetic construct
  REGION 1..23: note = Synthetic
MSFSSNVPQLFLLLVLLTNIVSG

SEQ ID NO: 108 - protein, 16 aa, synthetic construct
  REGION 1..16: note = Synthetic
MNLYLITLLFASLCSA

SEQ ID NO: 109 - protein, 18 aa, synthetic construct
  REGION 1..18: note = Synthetic
MRFSNFLTVSALLTGALG